(19) United States
(12) Patent Application Publication
(10) Pub. No.: US 2022/0071955 A1
(43) Pub. Date: Mar. 10, 2022

(54) Methods of Treatment, Prevention and Diagnosis

(71) Applicant: The Walter and Eliza Hall Institute of Medical Research, Melbourne, Victoria (AU)

(72) Inventors: Seth Masters, Victoria (AU); Chien-Hsiung Yu, Victoria (AU)

(21) Appl. No.: 17/290,187

(22) PCT Filed: Oct. 31, 2019
(86) PCT No.: PCT/AU2019/051201

§ 371 Date: Apr. 29, 2021

(30) Foreign Application Priority Data
Nov. 2, 2018 (AU) 2018904175

(51) Int. Cl. A61K 31/404; A61K 31/713; A61K 31/4184; A61K 31/7084; A61P 25/28

(52) U.S. Cl. CPC A61K 31/404; A61K 31/713; A61P 25/28; A61K 31/7084; A61K 31/4184

(57) ABSTRACT

The present invention relates to methods for treating or preventing TDP-43 proteinopathies, such as amyotrophic lateral sclerosis (ALS) and frontotemperal lobar degeneration (FTLD). The present invention also relates to methods of diagnosing TDP-43 proteinopathies.

### INCORPORATION BY REFERENCE OF SEQUENCE LISTING PROVIDED AS A TEXT FILE

A Sequence Listing is provided herewith in a text file, WEHI-008_SEQ_LIST_ST25, created on Nov. 23, 2021 and having a size of 5000 bytes. The contents of the text file are incorporated herein by reference in its entirety.

### FIELD OF THE INVENTION

The present invention relates to methods for treating or preventing TDP-43 proteinopathies, such as amyotrophic lateral sclerosis (ALS) and frontotemperal lobar degeneration (FTLD). The present invention also relates to methods for diagnosing TDP-43 proteinopathies.

### BACKGROUND OF THE INVENTION

Transactive response DNA binding protein 43 kDa (TDP-43) is a broadly expressed RNA-binding protein that plays an essential role in orchestrating post-transcriptional processing, including alternative splicing, transportation and mRNA stability for translation (Lee et al., 2012). Several non-specific mechanisms of cell stress and cellular toxicity are thought to be triggered by overexpressed, mislocalised and aggregated TDP-43, leading to disease. This includes reports that aggregated TDP-43 induces apoptosis (Rutherford et al., 2008; Sreedharan et al., 2008) and ER stress (Walker et al., 2013). Neurodegenerative diseases which are associated with aggregation and/or mislocalisation of TDP-43 are known as “TDP-43 proteinopathies”.

TDP-43-mediated neurodegeneration in diseases, such as ALS and frontotemperal lobar degeneration (FTLD), has been associated with hyperinflammatory responses, such as NF-κB related cytokine expression (Swarup et al., 2011), as well as elevated type I interferon (IFN) production (Wang et al., 2011). Interestingly, these inflammatory signals precede overt symptoms in mouse models of the disease (Wang et al., 2011). Despite these indications, molecular information regarding how TDP-43 causes neuroinflammation remains unavailable, limiting therapeutic options for treatment and prevention of TDP-43 proteinopathies. Furthermore, existing therapies for the treatment of TDP-43 proteinopathies are largely ineffective.

There is therefore a need for new methods for the treatment, prevention and diagnosis of TDP-43 proteinopathies.

### SUMMARY OF THE INVENTION

The present inventors have identified the previously unknown innate immune signalling pathway responsible for driving neurodegeneration in TDP-43 proteinopathies. Specifically, the present inventors surprisingly found that the cGAS-STING pathway is activated in response to cytosolic mtDNA which is released from mitochondria by TDP-43. The inventors have also found that symptoms and effects of TDP-43 proteinopathies can be reduced by inhibiting the cGAS-STING pathway.

Accordingly, in an aspect, the present invention provides a method of treating or preventing a TDP-43 proteinopathy in a subject, the method comprising administering a cGAS-STING pathway inhibitor to the subject.

The present invention also provides use of a cGAS-STING pathway inhibitor in the manufacture of a medicament for treatment or prevention of a TDP-43 proteinopathy in a subject.

The present invention also provides a cGAS-STING pathway inhibitor for use in the treatment or prevention of a TDP-43 proteinopathy in a subject.

In an embodiment, the TDP-43 proteinopathy is a neurodegenerative disease. Examples of TDP-43 proteinopathies which can be treated or prevented using a method of the invention include, but are not limited to, amyotrophic lateral sclerosis (ALS), motor neuron disease (MND), frontotemporal dementia (FTD), frontotemporal lobar degeneration (FTLD), Alzheimer's disease, Parkinson's disease, and inclusion body myositis (IBM).

In one embodiment, the TDP-43 proteinopathy is ALS. In one embodiment, the TDP-43 proteinopathy is FTLD.

In one embodiment, the ALS is sporadic ALS. Accordingly, in such embodiments the ALS is not inherited from a family member. In other embodiments, the ALS is familial ALS, i.e., it is caused by an inherited genetic mutation.

In one embodiment, the FTLD is FTLD with ubiquitin-positive inclusions (FTLD-U). FTLD-U is typically characterised by ubiquitin and TDP-43 positive, tau negative, FUS negative inclusions.

In one embodiment, the cGAS-STING pathway inhibitor is a STING inhibitor. The STING inhibitor may be a direct inhibitor, e.g., by interacting directly with STING or a nucleic acid (e.g., DNA or RNA) encoding STING, or an indirect inhibitor which reduces STING activity by interacting with another molecule.

In one embodiment, the STING inhibitor binds to STING. For instance, the STING inhibitor may bind to STING and sterically hinder binding of other molecules to STING, thereby inhibiting STING activity. Thus, in one embodiment, the STING inhibitor competitively inhibits cGAMP binding to STING. In one embodiment, the STING inhibitor covalently binds to STING. In one embodiment, the STING inhibitor covalently binds to Cys91 of STING.

In one embodiment, the STING inhibitor is a cyclic dinucleotide. STING is activated upon binding to cGAMP, which is a cyclic dinucleotide. Thus, cyclic dinucleotide STING inhibitors may act as cGAMP analogues which bind to and occupy the cGAMP binding site on STING while preventing STING from activating TPK1. In one embodiment, the cyclic dinucleotide is a cyclic purine dinucleotide. In one embodiment, the STING inhibitor is a cyclic purine dinucleotide described in WO2017093933.

In one embodiment, the STING inhibitor is a nitrofuran derivative. In one embodiment, the STING inhibitor blocks palmitoylation-induced clustering of STING.

In one embodiment, the STING inhibitor is a compound of the following formula

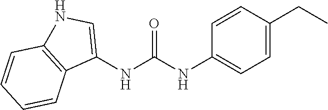 [SMILES: CCc1ccc(NC(=O)Nc2c[nH]c3ccccc23)cc1]

or a pharmaceutically acceptable salt, solvate, stereoisomer, tautomer, or prodrug thereof.

In some embodiments, the STING inhibitor is H-151, or a pharmaceutically acceptable salt, solvate, stereoisomer, tautomer, or prodrug thereof.

In one embodiment, the STING inhibitor is diclofenac, R(−)-2,10,11-Trihydroxyaporphine hydrobromide, Dipropyldopamine hydrobromide, (±)-trans-U-50488, 2,2′-Bipyridine, SP600125, Doxazosin mesylate, Mitoxantrone, MRS 2159, Nemadipine-A, (±)-PPHT hydrochloride, SMER28, Quinine, or Quisqualic acid.

In one embodiment, the STING inhibitor reduces the expression of STING. For instance, the STING inhibitor may reduce the expression of STING by reducing STING mRNA levels by, for example, RNA interference. Thus, in one embodiment, the STING inhibitor is an siRNA.

In one embodiment, the cGAS-STING pathway inhibitor is a cGAS inhibitor. The cGAS inhibitor may be a direct inhibitor, e.g., by interacting directly with cGAS or a nucleic acid (e.g., DNA or RNA) encoding cGAS, or an indirect inhibitor which reduces cGAS activity by interacting with another molecule.

In one embodiment, the cGAS inhibitor binds to cGAS. For instance, the cGAS inhibitor may bind to cGAS and sterically hinder binding of other molecules to cGAS, thereby inhibiting cGAS activity. In some embodiments, the cGAS inhibitor competitively inhibits cGAS binding to DNA.

In one embodiment, the cGAS inhibitor binds to the active site of cGAS. In one embodiment, the cGAS inhibitor inhibits cGAMP catalysis. For instance, the cGAS inhibitor may block access of ATP and GTP to the active site or may otherwise prevent catalytic residues in the active site of cGAS from catalyzing the conversion of ATP and GTP to cGAMP. In one embodiment, the cGAS inhibitor interacts with Arg364 and/or Tyr421 of cGAS.

In one embodiment, the cGAS inhibitor is a compound of the following formula

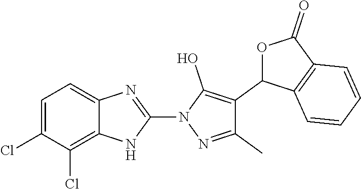 [SMILES: Cc1nn(-c2nc3ccc(Cl)c(Cl)c3[nH]2)c(O)c1C1OC(=O)c2ccccc21]

or a pharmaceutically acceptable salt, solvate, stereoisomer, tautomer, or prodrug thereof.

In one embodiment, the cGAS inhibitor is RU.521, or a pharmaceutically acceptable salt, solvate, stereoisomer, tautomer, or prodrug thereof.

In one embodiment, the cGAS inhibitor reduces the expression of cGAS. For instance, the cGAS inhibitor may reduce the expression of cGAS by reducing cGAS mRNA levels by, for example, RNA interference. Thus, in one embodiment, the cGAS inhibitor is an siRNA.

In one embodiment, the cGAS-STING pathway inhibitor is a cGAMP inhibitor. The cGAMP inhibitor may be a direct inhibitor, e.g., by interacting directly with cGAMP or an indirect inhibitor which reduces cGAMP-mediated activity by interacting with another molecule.

In one embodiment, the cGAMP inhibitor binds to cGAMP. For instance, the cGAMP inhibitor may bind to cGAMP and sterically hinder binding of other molecules to cGAMP. In some embodiments, the cGAMP inhibitor binds to cGAMP, thereby preventing cGAMP from binding to and activating STING. In one embodiment, the cGAMP inhibitor is a polypeptide comprising an antigen binding site which binds to cGAMP.

In one embodiment, the subject is a human. In one embodiment, the subject is a non-human mammal.

As the inventors surprisingly discovered, activation of the cGAS-STING pathway by TDP-43 mediated mtDNA release leads to expression of NF-κB related cytokines and type I interferon, which in turn cause neuroinflammation and thereby leading to neurodegenerative symptoms. Thus, in one embodiment, the cGAS-STING pathway inhibitor is administered in an amount sufficient to reduce or prevent an increase in TNFα and/or type I interferon expression.

In another aspect, the present invention provides a method for determining a likelihood of responsiveness to treatment with a cGAS-STING pathway inhibitor in a subject suffering from a neurodegenerative disease which is suspected to be a TDP-43 proteinopathy, the method comprising detecting activation of the cGAS-STING pathway in a sample from the subject, wherein activation of the cGAS-STING pathway indicates a higher likelihood of responsiveness to the treatment.

In another aspect, the present invention provides a method of diagnosing a subject with a TDP-43 proteinopathy, the method comprising detecting activation of the cGAS-STING pathway in a sample from the subject.

In one embodiment, detecting activation of the cGAS-STING pathway comprises measuring the level of cGAMP in the sample from the subject. In one embodiment, the level of cGAMP in the sample is measured using an ELISA.

In one embodiment, the sample is a blood sample. In another embodiment, the sample is a urine sample.

In another aspect, the present invention provides a kit comprising (a) a container comprising a cGAS-STING pathway inhibitor as described herein, optionally in a pharmaceutically acceptable carrier or diluent; and (b) a package insert with instructions for treating or preventing a TDP-43 proteinopathy in a subject.

Any embodiment herein shall be taken to apply mutatis mutandis to any other embodiment unless specifically stated otherwise. For instance, as the skilled person would understand examples of inhibitors and diseases outlined above for the methods of the invention equally apply to the use, the cGAS-STING pathway inhibitors for use, and the kits of the invention.

The present invention is not to be limited in scope by the specific embodiments described herein, which are intended for the purpose of exemplification only. Functionally-equivalent products, compositions and methods are clearly within the scope of the invention, as described herein.

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or group of compositions of matter.

### BRIEF DESCRIPTION OF THE ACCOMPANYING DRAWINGS

FIG. 1—Plasmids encoding wild-type (WT) TDP-43 or the ALS mutation (Q331K) were transiently overexpressed in mouse embryonic fibroblasts (MEFs) genetically deficient for different innate immune sensors. Production of Ifnb1 and Tnf was measured by qPCR after 48 hours, and was ablated only when cGAS or STING are genetically deficient.

FIG. 2—Inducible TDP-43 constructs (WT or Q331K) were transduced into WT or STING CRISPR KO Thp1 cells. 72 hours after doxycycline induction, qPCR for IFNB1 and TNF was performed.

FIG. 3—TDP-43 overexpressing Thp1 cells as in FIG. 2 were subjected to western blot analysis of inflammatory signaling pathways related to IFN and NF-κB.

FIG. 4—The cGAS inhibitor RU.521 and STING inhibitor H-151 prevent IFNB1 and TNF induction from TDP-43 overexpressing Thp1 cells used in FIG. 2.

FIG. 5—TDP-43-EGFP (WT, A315T or Q331K) and Flag-cGAS were transiently overexpressed in 293T cells, followed by extraction of DNA from Flag-immunoprecipitants. Direct qPCR reveals the presence of mtDNA (MT2 and MT3), but not DNA of nuclear origin (NC1 and NC2) bound to Flag-cGAS.

FIG. 6—Representative qPCR analysis of mitochondrial DNA depletion from Thp1 cells over two weeks' treatment in ethidium bromide. The mitochondrial genes (MT2 and MT3) were normalized to that of nuclear (NC1) to indicate the depletion relative to untreated cells.

FIG. 7—Human Thp1 cells with inducible TDP-43 (WT or Q331K) were depleted of mtDNA using EtBr (ρ⁰). 72 hrs after TDP-43 induction, qPCR for IFNB1 and TNF was performed.

FIG. 8—TDP-43 overexpressing Thp1 cells as in FIG. 7 were subjected to western blot analysis of inflammatory signaling pathways related to IFN and NF-κB.

FIG. 9—IFNB1 expression, measured by qPCR, in untreated and ρ⁰ thp-1 cells in response to stimulation with 2′3′-c-di-AM(PS)2(Rp, Rp) (20 μM) for 4 h or poly(dA:dT) (1 μg/ml) and ht-DNA (2 μg/ml) for 6 h.

FIG. 10—Quantification of super-resolution confocal microscopy reveals TDP-43 (FLAG-tagged) induced relocation of DNA (anti-DNA) from mitochondria (TOM20) into the cytoplasm.

FIG. 11—Flag-tagged TDP-43 (WT or Q331K) in WT or AGK knockout 293T were lysed and Flag-cGAS immunoprecipitated from which DNA was extracted directly amplified by qPCR to reveal the presence of mtDNA (MT1, MT2 and MT3) but not DNA of nuclear origin (NC1, NC2 and NC3).

FIG. 12—Representative western blot of cleaved caspase 3 in TDP-43-overexpressing MEFs 48 h post after doxycycline treatment.

FIG. 13—Plasmids encoding wild-type TDP-43 (WT) or the ALS mutation (Q331K) were transiently overexpressed in mouse embryonic fibroblasts (MEFs) that are genetically deficient for Bax and Bak. Production of Ifnb1 and Tnf was measured by qPCR after 48 hours.

FIG. 14—Human Thp1 cells with stably inducible TDP-43 (WT or Q331K) were treated with mitoSOX red 72 hrs after induction and subjected to FACS analysis (MFI: mean fluorescence intensity).

FIG. 15—Quantification of super-resolution confocal microscopy reveals TDP-43 (FLAG-tagged)-mediated DNA (anti-DNA) relocation from mitochondria (TOM20) into the cytoplasm was significantly reduced by inhibition of the mPTP (CsA, 25 μM) in MEFs.

FIG. 16—Inhibition of the mPTP (CsA, 25 μM) in 293T cells prevents mtDNA accumulation on FLAG-cGAS eluates.

FIG. 17—CRISPR-mediated genetic deletion of Ppif (encoding CypD) in MEFs, prevents mtDNA accumulation on FLAG-cGAS eluates.

FIG. 18—Inhibition of the mPTP (CsA, 25 μM) in 293T cells prevents Ifnb1 gene expression induced by TDP-43.

FIG. 19—CRISPR-mediated genetic deletion of Ppif (encoding CypD) in MEFs, prevents Ifnb1 gene expression induced by TDP-43

FIG. 20—Quantification of cGAMP in the cortex, spinal cords and serum of WT mice and mice that are transgenic for the human TDP-43 mutant allele A315T (n=2-5) at the experimental endpoint.

FIG. 21—TDP-43 mutant mice (n=17) develop progressive neurodegenerative disease that is lethal around 150 days. Heterozygous (n=9) or homozygous (n=12) loss of STING significantly increases lifespan.

FIG. 22—At 120 days, TDP-43 mutant mice exhibit significantly decreased gait impairment (n=6-21), which is greatly rectified by the genetic deletion of STING.

FIG. 23—At 120 days, TDP-43 mutant mice exhibit significantly decreased latency to fall in a rotarod test (n=3-8) which is greatly rectified by the genetic deletion of STING.

FIG. 24—Mouse movement in the Open Field (OF) test was video captured and analyzed by ImageJ and MouseMove. a) Cumulative trajectories of representative male mice from TDP-43^{T/+}STING^{+/+} (n=8), TDP-43^{T/+}STING^{+/-} (n=5) and TDP-43^{T/+}STING^{-/-} (n=8) at 130 days. b) Heterozygous and homozygous deletion of STING significantly restores motor coordination in TDP-43^{T/+} models of ALS relative to WT controls (n=12) in terms of distance travelled (m) and c) their fractional time spent stationary during 10 min OF test. Data are mean±SEM from 4 independent neurological behavior tests. P values were calculated using one-way ANOVA to WT mice.

FIG. 25—qPCR of inflammatory gene expression in the cortex and spinal cords reveals that increased levels of IFN and NF-κB dependent cytokines is greatly reduced due to the genetic deletion of STING (n=4-6).

FIG. 26—Representative nissl body staining (cresyl violet) of a coronal section through the brain of WT and TDP-43 mutant mice with and without the genetic deletion of STING.

FIG. 27—Quantification of cortical layer V neurons marked by a brown bar in FIG. 26.

FIG. 28—Quantification of cGAMP by ELISA from post mortem spinal cord samples of patients with ALS (n=16) or MS (n=12).

FIG. 29—When disease symptoms first appeared in TDP-43 mutant mice at 120 days of age, H-151 (3.75 mM) was administered i.p. every the other day for 4 weeks. This significantly improved latency to fall in a rotarod test (n=5). Data are means+/−SD and analysed by unpaired t-test. **P<0.01.

FIG. 30—qPCR of inflammatory gene expression in the cortex and spinal cords reveals that increased levels of IFN and NF-kB dependent cytokines is greatly reduced due to inhibition of STING using H-151 (n=2-3). TDP-43 mutant mice treated at 120 days of age with H-151 (3.75 mM) i.p. every the other day for 4 weeks.

FIG. 31—Quantification of mitochondrial destabilization markers a) mitoSOX red and b) Tetramethylrhodamine Methyl Ester (TMRM) by Flow cytometry (FACS) in iPSC-derived motor neurons. These demonstrate upregulation of ROS and loss of membrane potential (mΔψ) in ALS patient iPSC-derived motor neurons. Data are mean+/−SEM, pooled from 3 independent experiments and analysed using unpaired t-test between healthy control and ALS patients iPSC-MN lines, **P<0.01.

FIG. 32—The ROS inhibitors MitoQ and MitoTEMPO (0.1-1 μM) prevent IFNB1 and TNF gene induction in human iPSC-derived motor neurons from healthy controls and ALS patients carrying mutations in TDP-43. DMSO was used as solvent control (0). Data are mean+/−SEM, pooled from 3 independent experiments and analysed using unpaired t-test between healthy control and ALS patients iPSC-MN lines, *P<0.05.

FIG. 33—The cGAS inhibitor RU.521 (10 μM) and STING inhibitor H-151 (1 μM) prevent IFNB1 and TNF gene induction in human iPSC-derived motor neurons from healthy controls and ALS patients carrying mutations in TDP-43. DMSO was used as solvent control. Data are mean+/−SEM, pooled from 4 independent experiments and analysed using unpaired t-test between healthy control and ALS patients iPSC-MN lines, **P<0.01.

FIG. 34—Inhibition of STING mitigates ALS-associated cytotoxicity. a) Brightfield microscopy imaging and b) LDH cytotoxicity assay of healthy control and ALS patient iPSC-derived motor neurons following 4-week treatment with H-151 (1 μM) or DMSO as solvent control. Data are mean+/−SEM from 3 independent experiments and analysed using unpaired t-test, **P<0.01. Scale: 50 μm.

FIG. 35—Quantification of cGAMP by ELISA from cell lysates of iPSC-derived motor neurons shows active cGAS/STING signalling and potential for cGAMP as a biomarker in ALS patients carrying mutations in TDP-43. Data are mean+/−SEM, pooled from 5 independent experiments and analysed using unpaired t-test between healthy control and ALS patients iPSC-MN lines, ***P<0.001.

### DETAILED DESCRIPTION OF THE INVENTION

### General Techniques and Definitions

Unless specifically defined otherwise, all technical and scientific terms used herein shall be taken to have the same meaning as commonly understood by one of ordinary skill in the art (e.g., in neurology, immunology, molecular genetics, treatment of neurodegenerative disease, pharmacology, protein chemistry, and biochemistry).

Unless otherwise indicated, the techniques utilized in the present invention are standard procedures, well known to those skilled in the art. Such techniques are described and explained throughout the literature in sources such as, J. Perbal, A Practical Guide to Molecular Cloning, John Wiley and Sons (1984), J. Sambrook et al., Molecular Cloning: A Laboratory Manual, Cold Spring Harbour Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al., (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbour Laboratory, (1988), and J. E. Coligan et al., (editors) Current Protocols in Immunology, John Wiley & Sons (including all updates until present).

As used herein, the term about, unless stated to the contrary, refers to +/−10%, more preferably +/−5%, of the designated value.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

As used in this application, the term “or” is intended to mean an inclusive “or” rather than an exclusive “or”. That is, unless specified otherwise, or clear from context, “X employs A or B” is intended to mean any of the natural inclusive permutations. That is, if X employs A; X employs B; or X employs both A and B, then “X employs A or B” is satisfied under any of the foregoing instances. Further, at least one of A and B and/or the like generally means A or B or both A and B. In addition, the articles “a” and “an” as used in this application and the appended claims may generally be construed to mean “one or more” unless specified otherwise or clear from context to be directed to a singular form.

The term “cGAS” refers to cyclic GMP-AMP synthase, which upon binding of cytosolic DNA, catalyses cGAMP synthesis. The term “STING” refers to Stimulator of interferon genes (STING), which is also known as “transmembrane protein 173” (TMEM173) and “MPYS/MITA/ERIS”. The term “cGAMP”, as used herein, refers to cyclic guanosine monophosphate-adenosine monophosphate. cGAMP is also referred to as “cyclic GMP AMP” and “2′3′-cGAMP (cyclic [G(2′,5′)pA(3′,5′)p])”.

The term “cGAS-STING pathway inhibitor” as used herein, refers to any agent that can reduce signalling activity mediated by the cGAS-STING pathway. The cGAS-STING pathway inhibitor may be a direct inhibitor, e.g., by interacting directly with a component of the cGAS-STING pathway or an indirect inhibitor which reduces cGAS-STING signalling activity by interacting with another molecule. For instance, cGAS-STING pathway inhibitors can act to reduce expression of downstream inflammatory genes such as type I interferon and NF-κB related cytokines. Such inhibitors can act on any one or more components of the cGAS-STING pathway. Such inhibitors can act to reduce cGAS-STING signalling activity by any of a number of different modes of action including, but not limited to, inhibiting binding to their targets (e.g., cGAMP binding to STING), by blocking catalysis (e.g., by binding to the catalytic site of cGAS) or by reducing total levels of cGAS or STING protein such as by reducing mRNA levels. Examples of cGAS-STING pathway inhibitors include, for example, polynucleotides (e.g., siRNAs and mRNAs), small molecules, peptides, polypeptides (such as an antibody), or combinations thereof. A “cGAS” inhibitor is one which acts specifically on cGAS. Similarly, “STING inhibitors” and “cGAMP inhibitors” act specifically on STING and cGAMP respectively. In some embodiments one or more such inhibitors are used in combination.

The term “competitive inhibitor” or “competitively inhibits” as used herein, refers to a mode of inhibition of a target molecule in which an inhibitor binds to a functionally critical site on a target molecule itself (e.g., a ligand binding site) or on a ligand (e.g., a binding partner molecule) for the target molecule thereby sterically hindering interaction of the target molecule with the ligand. The competitive inhibitor may, but does not necessarily, have a higher affinity for the target molecule than the molecule with which it competes (e.g., the ligand).

The term “polypeptide” or “protein” as used herein, refer to a polymer of amino acids generally greater than about 50 amino acids in length.

As used herein, the term “binds” is in reference to a detectable interaction between two molecules, for example, between an inhibitor and its target. As used herein, the term “specifically binds” or “binds specifically”, or variations thereof, shall be taken to mean that a binding molecule associates more frequently, more rapidly, with greater duration and/or with greater affinity with a particular molecule than it does with alternative molecules. Generally, but not necessarily, reference to binding means specific binding, and each term shall be understood to provide explicit support for the other term.

As used herein, the term “subject” can be any animal. In one embodiment, the animal is a vertebrate. For example, the animal can be a mammal, avian, chordate, amphibian or reptile. Exemplary subjects include but are not limited to human, primate, livestock (e.g. sheep, cow, chicken, horse, donkey, pig), companion animals (e.g. dogs, cats), laboratory test animals (e.g. mice, rabbits, rats, guinea pigs, hamsters), captive wild animal (e.g. fox, deer). In one embodiment, the mammal is a human.

As the skilled person would understand, the inhibitors described herein will be administered in a therapeutically effective amount. The terms “effective amount” or “therapeutically effective amount” as used herein, refer to a sufficient amount of a cGAS-STING pathway inhibitor being administered which will relieve to some extent or prevent worsening of one or more of the symptoms of the disease or condition being treated. The result can be reduction or a prevention of progression of the signs, symptoms, or causes of a disease, or any other desired alteration of a biological system. For example, an “effective amount” for therapeutic uses is the amount of the cGAS-STING pathway inhibitor required to provide a clinically significant decrease in disease symptoms without undue adverse side effects. An appropriate “effective amount” in any individual case may be determined using techniques, such as a dose escalation study. The term “therapeutically effective amount” includes, for example, a prophylactically effective amount. An “effective amount” of a cGAS-STING pathway inhibitor is an amount effective to achieve a desired pharmacologic effect or therapeutic improvement without undue adverse side effects. It is understood that “an effect amount” or “a therapeutically effective amount” can vary from subject to subject, due to variation in metabolism of the compound of any of age, weight, general condition of the subject, the condition being treated, the severity of the condition being treated, and the judgment of the prescribing physician. By way of example only, therapeutically effective amounts may be determined by routine experimentation, including but not limited to a dose escalation clinical trial. Where more than one therapeutic agent is used in combination, a “therapeutically effective amount” of each therapeutic agent can refer to an amount of the therapeutic agent that would be therapeutically effective when used on its own, or may refer to a reduced amount that is therapeutically effective by virtue of its combination with one or more additional therapeutic agents.

The term “small molecule” as used herein, refers to a molecule having a molecular weight below 2000 daltons.

The terms “treating” or “treatment” as used herein, refer to both direct treatment of a subject by a medical professional (e.g., by administering a therapeutic agent to the subject), or indirect treatment, effected, by at least one party, (e.g., a medical doctor, a nurse, a pharmacist, or a pharmaceutical sales representative) by providing instructions, in any form, that (i) instruct a subject to self-treat according to a claimed method (e.g., self-administer a drug) or (ii) instruct a third party to treat a subject according to a claimed method. Also encompassed within the meaning of the term “treating” or “treatment” are prevention or reduction of the disease to be treated, e.g., by administering a therapeutic at a sufficiently early phase of disease to prevent or slow its progression.

The terms “preventing” or “prevention” as used herein, refer to the process of administering a compound to a subject with the aim of preventing the onset of the disease or symptoms thereof. These terms encompass both total prevention of the disease and temporary prevention. Specifically, the methods described herein can be used to prevent a TDP-43 proteinopathy completely or to delay the onset of the TDP-43 proteinopathy or symptoms thereof. Such preventative methods are particularly useful for subjects with a predisposition to a TDP-43 proteinopathy, but who have not yet suffered from symptoms of the TDP-43 proteinopathy.

The terms “co-administration” or “administered in combination” or the like, as used herein, are meant to encompass administration of the selected therapeutic agents to a single subject, and are intended to include treatment regimens in which the agents are administered by the same or different route of administration or at the same or different time.

### TDP-43 Proteinopathies

The methods described herein include treating or preventing a TDP-43 proteinopathy in a subject, the method comprising administering a cGAS-STING pathway inhibitor to the subject.

The cGAS-STING pathway is a component of the innate immune system that functions to detect the presence of cytosolic DNA and, in response, triggers expression of inflammatory genes that can lead to senescence or to the activation of defence mechanisms. DNA is normally found in the nucleus of the cell and thus localization of DNA to the cytosol is normally associated with tumorigenesis or viral infection.

Upon binding DNA, cGAS triggers reaction of GTP and ATP to form cyclic GMP-AMP (cGAMP). cGAMP binds to STING which triggers phosphorylation of IRF3 via TBK1. Phosphorylated IRF3 can then trigger transcription of inflammatory genes such as NF-κB related cytokines and type I interferon. The cGAS-STING pathway therefore acts to detect cytosolic DNA and induce an immune response.

The present inventors have surprisingly found that overexpressed, mutant, and/or mislocalised TDP-43 causes release of mitochondrial DNA (mtDNA) to the cytosol, which in turn activates the cGAS pathway leading to neuroinflammation in TDP-43 proteinopathies, such as ALS, FTLD, Alzheimer's disease, and Parkinson disease. Thus, for the first time the present inventors have identified the previously unknown innate immune sensor pathway responsible for neurodegeneration in such diseases. This offers new avenues for therapeutic intervention of the pathway for treatment and prevention of TDP-43 proteinopathies.

Without being bound by theory, it is believed that inhibiting the cGAS-STING pathway reduces neurodegeneration caused, at least in part, by cGAS-STING mediated inflammation to achieve a therapeutic effect in TDP-43 proteinopathies.

As used herein, the phrase “TDP-43 proteinopathies” refers to diseases associated with mislocalisation and/or aggregation of TDP-43 in the cytoplasm of neurons. In an embodiment, the TDP-43 proteinopathy is a neurodegenerative disease. Exemplary TDP-43 proteinopathies include motor neurone disease (MND) such as amyotrophic lateral sclerosis (ALS), dementia, frontotemporal lobar degeneration (FTLD) including frontotemporal lobar degeneration with ubiquitin/TDP-43 positive and tau-negative inclusions (FTLD-U), frontotemporal dementia (FTD), Parkinson's disease (PD), Guam parkinsonism-dementia, or a Lewy body-related disease such as Alzheimer's disease (AD).

In one embodiment the TDP-43 proteinopathy is MND. As used herein, “MND” refers to a class of diseases including ALS, spinal muscular atrophy and spinal and bulbar muscular atrophy (SBMA, or Kennedy's disease).

In one embodiment, the TDP-43 proteinopathy is ALS. ALS is characterised by degeneration of the upper and lower motor neurons, leading to progressive muscle atrophy and wasting, weakness and spasticity. Approximately 10% of ALS cases have a positive family history associated with mutations in genes such as superoxide dismutase (SODI), dynactin (DCTNI), and vesicle trafficking protein (VAPB). Such cases are referred to as “familial ALS”. The remaining cases are referred to as “sporadic” ALS. In one embodiment, the ALS is sporadic ALS. In other embodiments, the ALS is familial ALS.

In one embodiment, the TDP-43 proteinopathy is FTD. In one embodiment, the TDP-43 proteinopathy is FTLD. Frontotemporal lobar degeneration (FTLD) is a pathological process that occurs in FTD and is characterised by degeneration of neurons in the superficial frontal cortex and anterior temporal lobes. FTLD can be categorised into two main groups: cases with tau-positive pathology known as tauopathies (FTLD-tau), and the more frequent cases with ubiquitin and TDP-43 inclusions known as FTLD-U. Thus, in some embodiments, the TDP-43 proteinopathy is FTLD-U.

In one embodiment, the TDP-43 proteinopathy is inclusion body myositis (IBM). IBM is a progressive muscle disorder characterized by muscle inflammation, weakness, and atrophy (wasting). It is a type of inflammatory myopathy and typically develops in adulthood, usually after age 50.

cGAS-STING Pathway Inhibitors

cGAS-STING pathway inhibition, as used herein, refers to reducing one or more of net cGAS or STING expression, net cGAS or STING protein levels, or cGAS-STING signalling activity (e.g., inhibition of expression of inflammatory cytokines). Inhibition of the cGAS-STING pathway may include at least about a 10% to a 100% reduction in the level of cGAS-STING activity in the presence of, or resulting from, a given dose of the cGAS-STING pathway inhibitor relative to cGAS-STING activity level in its absence, e.g., a 10%, 20%, 30%, 40%, 50%, 60%, 70%, 80%, 85%, 90%, 95%, or another percent reduction in cGAS-STING activity from about 10% to about 100%.

cGAS-STING pathway inhibition can be quantified, for example, by measuring expression and/or protein levels of NF-κB and/or type I interferon related cytokines. cGAS-STING pathway inhibition can also be quantified using an assay for cGAMP, such as those described in Hall et al. (2017). Suitable methods for measuring the levels of NF-κB and/or type I interferon related cytokines will be known in the art and include qPCR and enzyme-linked immunosorbent assay (ELISA). For example, type 1 interferon production can be measured using the assay described in Seeds and Miller (2011). NF-κB cytokine production can be assessed using commercially available assays for quantifying TNFα such as the “Human TNF alpha Assay Kit” available from Cisbio (cat no. 62HTNFAPEG) or the “TNF alpha Human ELISA Kit” available from ThermoFisher (cat no. KHC3011). Other methods for quantifying cGAS-STING mediated signalling which can be used for quantifying cGAS-STING pathway inhibition are described in the Examples.

Examples of types of cGAS-STING pathway inhibitors useful for the invention include, but are not limited to, a small molecule, a polynucleotide, a polypeptide, or a peptide.

### Small Molecules

In some embodiments, the cGAS-STING pathway inhibitor is a small molecule. In some embodiments, the small molecule binds to cGAS or STING and reduces their activity, e.g., the catalysis of cGAMP by cGAS or activation of TBK1 by STING. Suitable small molecule cGAS-STING pathway inhibitors for use in the invention can be identified using screening methods that are routine in the art.

In some embodiments, the small molecule that is administered may be a precursor compound, commonly referred to as a “prodrug” which is inactive or comparatively poorly active, but which, following administration, is converted (i.e., metabolised) to an active cGAS-STING pathway inhibitor. In those embodiments, the compound that is administered may be referred to as a prodrug. Alternatively, or in addition, the compounds that are administered may be metabolized to produce active metabolites which have activity in reducing cGAS-STING mediated signalling activity. The use of such active metabolites is also within the scope of the present disclosure.

Depending on the substituents present in the compound, the compound may optionally be present in the form of a pharmaceutically acceptable salt. Salts of compounds which are suitable for use in the described methods are those in which a counter-ion is pharmaceutically acceptable. Suitable salts include those formed with organic or inorganic acids or bases. In particular, suitable salts formed with acids include those formed with mineral acids, strong organic carboxylic acids, such as alkane carboxylic acids of 1 to 4 carbon atoms which are unsubstituted or substituted, for example, by halogen, such as saturated or unsaturated dicarboxylic acids, such as hydroxycarboxylic acids, such as amino acids, or with organic sulfonic acids, such as (C₁₋₄)-alkyl- or aryl-sulfonic acids which are substituted or unsubstituted, for example by halogen. Pharmaceutically acceptable acid addition salts include those formed from hydrochloric, hydrobromic, sulphuric, nitric, citric, tartaric, acetic, phosphoric, lactic, pyruvic, acetic, trifluoroacetic, succinic, perchloric, fumaric, maleic, glycolic, lactic, salicylic, oxaloacetic, methanesulfonic, ethanesulfonic, p-toluenesulfonic, formic, benzoic, malonic, naphthalene-2-sulfonic, benzenesulfonic, isethionic, ascorbic, malic, phthalic, aspartic, and glutamic acids, lysine and arginine. Pharmaceutically acceptable base salts include ammonium salts, alkali metal salts, for example those of potassium and sodium, alkaline earth metal salts, for example those of calcium and magnesium, and salts with organic bases, for example dicyclohexylamine, N-methyl-D-glucomine, morpholine, thiomorpholine, piperidien, pyrrolidine, a mono-, di- or tri-lower alkylamine, for example ethyl-, tbutyl-, diethyl-, diisopropyl-, triethyl-, tributyl- or dimethyl-propylamine, or a mono-, di- or trihydroxy lower alkylamine, for example mono-, di- or triethanolamine. Corresponding internal salts may also be formed.

Those skilled in the art of organic and/or medicinal chemistry will appreciate that many organic compounds can form complexes with solvents in which they are reacted or from which they are precipitated or crystallised. These complexes are known as “solvates”. For example, a complex with water is known as a “hydrate”. Solvates, such as hydrates, exist when the drug substance incorporates solvent, such as water, in the crystal lattice in either stoichiometric or non-stoichiometric amounts. Drug substances are routinely screened for the existence of solvates such as hydrates since these may be encountered at any stage. Accordingly, it will be understood that the compounds useful for the present invention may be present in the form of solvates, such as hydrates. Solvated forms of the compounds which are suitable for use in the invention are those wherein the associated solvent is pharmaceutically acceptable. For example, a hydrate is an example of a pharmaceutically acceptable solvate.

The compounds useful for the present invention may be present in amorphous form or crystalline form. Many compounds exist in multiple polymorphic forms, and the use of the compounds in all such forms is encompassed by the present disclosure. Small molecules useful for the present disclosure can be identified using standard procedures such as screening a library of candidate compounds for binding to cGAS or STING, and then determining if any of the compounds which bind reduce cGAS-STING mediated signalling activity. In some embodiments, screening for a compound for use in the invention comprises assessing whether the compound inhibits cGAS-STING signalling activity in cells. Small molecules useful for the present invention can also be identified using procedures for in silico screening, which can include screening of known library compounds, to identify candidates which reduce cGAS-STING signalling activity.

With regard to the features of the compounds according to any one of Formula (I), (II), (IIa), (IIb), (IIc), (IId), (IIe), (IIf), (IIIa), (IIIb), (IIIc), (IV), (IVa), (IVb), (IVc), and (V), definitions are provided for the following terms:

The term “carbocyclic” represents a monocyclic or polycyclic ring system wherein the ring atoms are all carbon atoms, e.g., of about 3 to about 20 carbon atoms, and which may be aromatic, non-aromatic, saturated, or unsaturated, and may be substituted and/or contain fused rings. Examples of such groups include aryl groups such as benzene, saturated groups such as cyclopentyl, or fully or partially hydrogenated phenyl, naphthyl and fluorenyl. It will be appreciated that the polycyclic ring system includes bicyclic and tricyclic ring systems.

“Heterocyclic” represents a monocyclic or polycyclic ring system wherein the ring atoms are provided by at least two different elements, typically a combination of carbon and one or more of nitrogen, sulphur and oxygen, although may include other elements for ring atoms such as selenium, boron, phosphorus, bismuth and silicon, and wherein the ring system is about 3 to about 20 atoms, and which may be aromatic such as a “heteroaryl” group, non-aromatic, saturated, or unsaturated, and may be substituted and/or contain fused rings. For example, the heterocyclic may be (i) an optionally substituted cycloalkyl or cycloalkenyl group, e.g., of about 3 to about 20 ring members, which may contain one or more heteroatoms such as nitrogen, oxygen, or sulfur (examples include pyrrolidinyl, morpholino, thiomorpholino, or fully or partially hydrogenated thienyl, furyl, pyrrolyl, thiazolyl, oxazolyl, oxazinyl, thiazinyl, pyridyl and azepinyl); (ii) an optionally substituted partially saturated monocyclic or polycyclic ring system in which an aryl (or heteroaryl) ring and a heterocyclic group are fused together to form a cyclic structure (examples include chromanyl, dihydrobenzofuryl and indolinyl); or (iii) an optionally substituted fully or partially saturated polycyclic fused ring system that has one or more bridges (examples include quinuclidinyl and dihydro-1,4-epoxynaphthyl). It will be appreciated that the polycyclic ring system includes bicyclic and tricyclic ring systems.

As will be understood, an “aromatic” group means a cyclic group having 4m+2π electrons, where m is an integer equal to or greater than 1. As used herein, “aromatic” is used interchangeably with “aryl” to refer to an aromatic group, regardless of the valency of aromatic group.

“Aryl” whether used alone, or in compound words such as arylalkyl, aryloxy or arylthio, represents: (i) an optionally substituted mono- or polycyclic aromatic carbocyclic moiety, e.g., of about 6 to about 20 carbon atoms, such as phenyl, naphthyl or fluorenyl; or, (ii) an optionally substituted partially saturated polycyclic carbocyclic aromatic ring system in which an aryl and a cycloalkyl or cycloalkenyl group are fused together to form a cyclic structure such as a tetrahydronaphthyl, indenyl, indanyl or fluorene ring. It will be appreciated that the polycyclic ring system includes bicyclic and tricyclic ring systems.

A “hetaryl”, “heteroaryl” or heteroaromatic group, is an aromatic group or ring containing one or more heteroatoms, such as N, O, S, Se, Si or P. As used herein, “heteroaromatic” is used interchangeably with “hetaryl” or “heteroaryl”, and a heteroaryl group refers to monovalent aromatic groups, bivalent aromatic groups and higher multivalency aromatic groups containing one or more heteroatoms. For example, “heteroaryl” whether used alone, or in compound words such as heteroaryloxy represents: (i) an optionally substituted mono- or polycyclic aromatic organic moiety, e.g., of about 5 to about 20 ring members in which one or more of the ring members is/are element(s) other than carbon, for example nitrogen, oxygen, sulfur or silicon; the heteroatom(s) interrupting a carbocyclic ring structure and having a sufficient number of delocalized π electrons to provide aromatic character, provided that the rings do not contain adjacent oxygen and/or sulfur atoms. Typical 6-membered heteroaryl groups are pyrazinyl, pyridazinyl, pyrazolyl, pyridyl and pyrimidinyl. All regioisomers are contemplated, e.g., 2-pyridyl, 3-pyridyl and 4-pyridyl. Typical 5-membered heteroaryl rings are furyl, imidazolyl, oxazolyl, isoxazolyl, isothiazolyl, oxadiazolyl, pyrrolyl, 1,3,4-thiadiazolyl, thiazolyl, thienyl, triazolyl, and silole. All regioisomers are contemplated, e.g., 2-thienyl and 3-thienyl. Bicyclic groups typically are benzo-fused ring systems derived from the heteroaryl groups named above, e.g., benzofuryl, benzimidazolyl, benzthiazolyl, indolyl, indolizinyl, isoquinolyl, quinazolinyl, quinolyl and benzothienyl; or, (ii) an optionally substituted partially saturated polycyclic heteroaryl ring system in which a heteroaryl and a cycloalkyl or cycloalkenyl group are fused together to form a cyclic structure such as a tetrahydroquinolyl or pyrindinyl ring. It will be appreciated that the polycyclic ring system includes bicyclic and tricyclic ring systems.

The term “optionally fused” means that a group is either fused by another ring system or unfused, and “fused” refers to one or more rings that share at least one common ring atom with one or more other rings. The fusing may be provided a single common ring atom, for example a spiro compound. The fusing may be provided by at least two common atoms. Fusing may be provided by one or more carbocyclic, heterocyclic, aryl or hetaryl rings, as defined herein, or be provided by substituents of rings being joined together to form a further ring system. The fused ring may have between 5 and 10 ring atoms in size, for example a 5, 6 or 7 membered ring. The fused ring may be fused to one or more other rings, and may for example contain 1 to 4 rings.

The term “optionally substituted” means that a functional group is either substituted or unsubstituted, at any available position. Substitution can be with one or more functional groups selected from, e.g., alkyl, alkenyl, alkynyl, cycloalkyl, cycloalkenyl, aryl, heterocyclyl, heteroaryl, formyl, alkanoyl, cycloalkanoyl, aroyl, heteroaroyl, carboxyl, alkoxycarbonyl, cycloalkyloxycarbonyl, aryloxycarbonyl, heterocyclyloxycarbonyl, heteroaryloxycarbonyl, alkylaminocarbonyl, cycloalkylaminocarbonyl, arylaminocarbonyl, heterocyclylaminocarbonyl, heteroarylaminocarbonyl, cyano, alkoxy, cycloalkoxy, aryloxy, heterocyclyloxy, heteroaryloxy, alkanoate, cycloalkanoate, aryloate, heterocyclyloate, heteroaryloate, alkylcarbonylamino, cycloalkylcarbonylamino, arylcarbonylamino, heterocyclylcarbonylamino, heteroarylcarbonylamino, nitro, alkylthio, cycloalkylthio, arylthio, heterocyclylthio, heteroarylthio, alkylsulfonyl, cycloalkylsulfonyl, arylsulfonyl, heterocyclysulfonyl, heteroarylsulfonyl, hydroxyl, halo, haloalkyl, haloaryl, haloheterocyclyl, haloheteroaryl, haloalkoxy, haloalkylsulfonyl, silylalkyl, alkenylsilylalkyl, and alkynylsilylalkyl. It will be appreciated that other groups not specifically described may also be used.

The term “halo” or “halogen” whether employed alone or in compound words such as haloalkyl, haloalkoxy or haloalkylsulfonyl, represents fluorine, chlorine, bromine or iodine. Further, when used in compound words such as haloalkyl, haloalkoxy or haloalkylsulfonyl, the alkyl may be partially halogenated or fully substituted with halogen atoms which may be independently the same or different. Examples of haloalkyl include, without limitation, —CH₂CH₂F, —CF₂CF₃ and —CH₂CHFCl. Examples of haloalkoxy include, without limitation, —OCHF₂, —OCF₃, —OCH₂CCl₃, —OCH₂CF₃ and —OCH₂CH₂CF₃. Examples of haloalkylsulfonyl include, without limitation, —SO₂CF₃, —SO₂CCl₃, —SO₂CH₂CF₃ and —SO₂CF₂CF₃.

“Alkyl” whether used alone, or in compound words such as alkoxy, alkylthio, alkylamino, dialkylamino or haloalkyl, represents straight or branched chain hydrocarbons ranging in size from one to about 20 carbon atoms, or more. Thus alkyl moieties include, unless explicitly limited to smaller groups, moieties ranging in size, for example, from one to about 6 carbon atoms or greater, such as, methyl, ethyl, n-propyl, iso-propyl and/or butyl, pentyl, hexyl, and higher isomers, including, e.g., those straight or branched chain hydrocarbons ranging in size from about 6 to about 20 carbon atoms, or greater.

“Alkenyl” whether used alone, or in compound words such as alkenyloxy or haloalkenyl, represents straight or branched chain hydrocarbons containing at least one carbon-carbon double bond, including, unless explicitly limited to smaller groups, moieties ranging in size from two to about 6 carbon atoms or greater, such as, methylene, ethylene, 1-propenyl, 2-propenyl, and/or butenyl, pentenyl, hexenyl, and higher isomers, including, e.g., those straight or branched chain hydrocarbons ranging in size, for example, from about 6 to about 20 carbon atoms, or greater.

“Alkynyl” whether used alone, or in compound words such as alkynyloxy, represents straight or branched chain hydrocarbons containing at least one carbon-carbon triple bond, including, unless explicitly limited to smaller groups, moieties ranging in size from, e.g., two to about 6 carbon atoms or greater, such as, ethynyl, 1-propynyl, 2-propynyl, and/or butynyl, pentynyl, hexynyl, and higher isomers, including, e.g., those straight or branched chain hydrocarbons ranging in size from, e.g., about 6 to about 20 carbon atoms, or greater.

“Cycloalkyl” represents a mono- or polycarbocyclic ring system of varying sizes, e.g., from about 3 to about 20 carbon atoms, e.g., cyclopropyl, cyclobutyl, cyclopentyl, cyclohexyl or cycloheptyl. The term cycloalkyloxy represents the same groups linked through an oxygen atom such as cyclopentyloxy and cyclohexyloxy. The term cycloalkylthio represents the same groups linked through a sulfur atom such as cyclopentylthio and cyclohexylthio.

“Cycloalkenyl” represents a non-aromatic mono- or polycarbocyclic ring system, e.g., of about 3 to about 20 carbon atoms containing at least one carbon-carbon double bond, e.g., cyclopentenyl, cyclohexenyl or cycloheptenyl. The term “cycloalkenyloxy” represents the same groups linked through an oxygen atom such as cyclopentenyloxy and cyclohexenyloxy. The term “cycloalkenylthio” represents the same groups linked through a sulfur atom such as cyclopentenylthio and cyclohexenylthio.

“Cycloalkynyl” represents a non-aromatic mono- or polycarbocyclic ring system, e.g., of about 3 to about 20 carbon atoms containing at least one carbon-carbon double bond, e.g., cyclopentenyl, cyclohexenyl or cycloheptenyl. The term “cycloalkenyloxy” represents the same groups linked through an oxygen atom such as cyclopentenyloxy and cyclohexenyloxy. The term “cycloalkenylthio” represents the same groups linked through a sulfur atom such as cyclopentenylthio and cyclohexenylthio.

“Formyl” represents a —CHO moiety.

“Alkanoyl” represents a —C(═O)-alkyl group in which the alkyl group is as defined supra. In a particular embodiment, an alkanoyl ranges in size from about C₂-C₂₀. One example is acyl.

“Aroyl” represents a —C(═O)-aryl group in which the aryl group is as defined supra. In a particular embodiment, an aroyl ranges in size from about C₇-C₂₀. Examples include benzoyl and 1-naphthoyl and 2-naphthoyl.

“Heterocycloyl” represents a —C(═O)-heterocyclyl group in which the heterocylic group is as defined supra. In a particular embodiment, an heterocycloyl ranges in size from about C₄-C₂₀.

“Heteroaroyl” represents a —C(═O)-heteroaryl group in which the heteroaryl group is as defined supra. In a particular embodiment, a heteroaroyl ranges in size from about C₆-C₂₀. An example is pyridylcarbonyl.

“Carboxyl” represents a —CO₂H moiety.

“Oxycarbonyl” represents a carboxylic acid ester group —CO₂R which is linked to the rest of the molecule through a carbon atom.

“Alkoxycarbonyl” represents an —CO₂-alkyl group in which the alkyl group is as defined supra. In a particular embodiment, an alkoxycarbonyl ranges in size from about C₂-C₂₀. Examples include methoxycarbonyl and ethoxycarbonyl.

“Aryloxycarbonyl” represents an —CO₂-aryl group in which the aryl group is as defined supra. Examples include phenoxycarbonyl and naphthoxycarbonyl.

“Heterocyclyloxycarbonyl” represents a —CO₂-heterocyclyl group in which the heterocyclic group is as defined supra.

“Heteroaryloxycarbonyl” represents a —CO-heteroaryl group in which the heteroaryl group is as defined supra.

“Aminocarbonyl” represents a carboxylic acid amide group —C(═O)NHR or —C(═O)NR₂ which is linked to the rest of the molecule through a carbon atom.

“Alkylaminocarbonyl” represents a —C(═O)NHR or —C(═O)NR₂ group in which R is an alkyl group as defined supra.

“Arylaminocarbonyl” represents a —C(═O)NHR or —C(═O)NR₂ group in which R is an aryl group as defined supra.

“Heterocyclylaminocarbonyl” represents a —C(═O)NHR or —C(═O)NR₂ group in which R is a heterocyclic group as defined supra. In certain embodiments, NR₂ is a heterocyclic ring, which is optionally substituted.

“Heteroarylaminocarbonyl” represents a —C(═O)NHR or —C(═O)NR₂ group in which R is a heteroaryl group as defined supra. In certain embodiments, NR₂ is a heteroaryl ring, which is optionally substituted.

“Cyano” represents a —CN moiety.

“Hydroxyl” represents a —OH moiety.

“Alkoxy” represents an —O-alkyl group in which the alkyl group is as defined supra. Examples include methoxy, ethoxy, n-propoxy, iso-propoxy, and the different butoxy, pentoxy, hexyloxy and higher isomers.

“Aryloxy” represents an —O-aryl group in which the aryl group is as defined supra. Examples include, without limitation, phenoxy and naphthoxy.

“Alkenyloxy” represents an —O-alkenyl group in which the alkenyl group is as defined supra. An example is allyloxy.

“Heterocyclyloxy” represents an —O-heterocyclyl group in which the heterocyclic group is as defined supra.

“Heteroaryloxy” represents an —O-heteroaryl group in which the heteroaryl group is as defined supra. An example is pyridyloxy.

“Alkanoate” represents an —OC(═O)—R group in which R is an alkyl group as defined supra.

“Aryloate” represents a —OC(═O)—R group in which R is an aryl group as defined supra.

“Heterocyclyloate” represents an —OC(═O)—R group in which R is a heterocyclic group as defined supra.

“Heteroaryloate” represents an —OC(═O)—R group in which P is a heteroaryl group as defined supra.

“Amino” represents an —NH₂ moiety.

“Alkylamino” represents an —NHR or —NR₂ group in which R is an alkyl group as defined supra. Examples include, without limitation, methylamino, ethylamino, n-propylamino, isopropylamino, and the different butylamino, pentylamino, hexylamino and higher isomers.

“Arylamino” represents an —NHR or —NR₂ group in which R is an aryl group as defined supra. An example is phenylamino.

“Heterocyclylamino” represents an —NHR or —NR₂ group in which R is a heterocyclic group as defined supra. In certain embodiments, NR₂ is a heterocyclic ring, which is optionally substituted.

“Heteroarylamino” represents a —NHR or —NR₂ group in which R is a heteroaryl group as defined supra. In certain embodiments, NR₂ is a heteroaryl ring, which is optionally substituted.

“Carbonylamino” represents a carboxylic acid amide group —NHC(═O)R that is linked to the rest of the molecule through a nitrogen atom.

“Alkylcarbonylamino” represents a —NHC(═O)R group in which R is an alkyl group as defined supra.

“Arylcarbonylamino” represents an —NHC(═O)R group in which R is an aryl group as defined supra.

“Heterocyclylcarbonylamino” represents an —NHC(═O)R group in which R is a heterocyclic group as defined supra.

“Heteroarylcarbonylamino” represents an —NHC(═O)R group in which R is a heteroaryl group as defined supra.

“Nitro” represents a —NO₂ moiety.

“Alkylthio” represents an —S-alkyl group in which the alkyl group is as defined supra. Examples include, without limitation, methylthio, ethylthio, n-propylthio, iso propylthio, and the different butylthio, pentylthio, hexylthio and higher isomers.

“Arylthio” represents an —S-aryl group in which the aryl group is as defined supra. Examples include phenylthio and naphthylthio.

“Heterocyclylthio” represents an —S-heterocyclyl group in which the heterocyclic group is as defined supra.

“Heteroarylthio” represents an —S-heteroaryl group in which the heteroaryl group is as defined supra.

“Sulfonyl” represents an —SO₂R group that is linked to the rest of the molecule through a sulfur atom.

“Alkylsulfonyl” represents an —SO₂-alkyl group in which the alkyl group is as defined supra.

“Arylsulfonyl” represents an —SO₂-aryl group in which the aryl group is as defined supra.

“Heterocyclylsulfonyl” represents an —SO₂-heterocyclyl group in which the heterocyclic group is as defined supra.

“Heteoarylsulfonyl” presents an —SO₂-heteroaryl group in which the heteroaryl group is as defined supra.

“Aldehyde” represents a —C(═O)H group.

“Alkanal” represents an alkyl-(C═O)H group in which the alkyl group is as defined supra.

“Alkylsilyl” presents an alkyl group that is linked to the rest of the molecule through the silicon atom, which may be substituted with up to three independently selected alkyl groups in which each alkyl group is as defined supra.

“Alkenylsilyl” presents an alkenyl group that is linked to the rest of the molecule through the silicon atom, which may be substituted with up to three independently selected alkenyl groups in which each alkenyl group is as defined supra.

“Alkynylsilyl” presents an alkynyl group that is linked to the rest of the molecule through the silicon atom, which may be substituted with up to three independently selected alkynyl groups in which each alkenyl group is as defined supra.

“Aryl” refers to a carbocyclic aromatic group. Examples of aryl groups include, but are not limited to, phenyl, naphthyl and anthracenyl. A carbocyclic aromatic group or a heterocyclic aromatic group can be unsubstituted or substituted with one or more groups including, but not limited to, —C₁-C₈ alkyl, —O—(C₁-C₈ alkyl), -aryl, —C(O)R′, —OC(O)R′, —C(O)OR′, —C(O)NH₂, —C(O)NHR′, —C(O)N(R′)₂—NHC(O)R′, —S(O)₂R′, —S(O)R′, —OH, -halogen, —N₃, —NH₂, —NH(R′), —N(R′)₂ and —CN; wherein each R′ is independently selected from H, —C₁-C₈ alkyl and aryl.

The term “C₁₋₁₀alkyl,” as used herein refers to a straight chain or branched, saturated or unsaturated hydrocarbon having from 1 to 10 carbon atoms. Representative “C₁₋₁₀alkyl” groups include, but are not limited to, -methyl, -ethyl, -n-propyl, -n-butyl, -n-pentyl, -n-hexyl, -n-heptyl, -n-octyl, -n-nonyl and -n-decyl; while branched C₁-C₈ alkyls include, but are not limited to, -isopropyl, -sec-butyl, -isobutyl, -tert-butyl, -isopentyl, 2-methylbutyl, unsaturated C₁-C₈ alkyls include, but are not limited to, -vinyl, -allyl, -1-butenyl, -2-butenyl, -isobutylenyl, -1-pentenyl, -2-pentenyl, -3-methyl-1-butenyl, -2-methyl-2-butenyl, -2,3-dimethyl-2-butenyl, 1-hexyl, 2-hexyl, 3-hexyl, -acetylenyl, -propynyl, -1-butynyl, -2-butynyl, -1-pentynyl, -2-pentynyl, -3-methyl-1 butynyl, methyl, ethyl, propyl, isopropyl, n-butyl, isobutyl, sec-butyl, tert-butyl, n-pentyl, isopentyl, neopentyl, n-hexyl, isohexyl, 2-methylpentyl, 3-methylpentyl, 2,2-dimethylbutyl, 2,3-dimethylbutyl, 2,2-dimethylpentyl, 2,3-dimethylpentyl, 3,3-dimethylpentyl, 2,3,4-trimethylpentyl, 3-methylhexyl, 2,2-dimethylhexyl, 2,4-dimethylhexyl, 2,5-dimethylhexyl, 3,5-dimethylhexyl, 2,4-dimethylpentyl, 2-methylheptyl, 3-methylheptyl, n-heptyl, isoheptyl, n-octyl, and isooctyl. A C₁-C₈ alkyl group can be unsubstituted or substituted with one or more groups including, but not limited to, —C₁-C₈ alkyl, —O—(C₁-C₈ alkyl), -aryl, —C(O)R′, —OC(O)R′, —C(O)OR′, —C(O)NH₂, —C(O)NHR′, —C(O)N(R′)₂—NHC(O)R′, —SO₃R′, —S(O)₂R′, —S(O)R′, —OH, -halogen, —N₃, —NH₂, —NH(R′), —N(R′)₂ and —CN; where each R′ is independently selected from H, —C₁-C₈ alkyl and aryl.

A “C₃₋₁₂carbocyclyl” is a 3-, 4-, 5-, 6-, 7- or 8-membered saturated or unsaturated non-aromatic carbocyclic ring. Representative C₃₋₁₂carbocycles include, but are not limited to, -cyclopropyl, -cyclobutyl, -cyclopentyl, -cyclopentadienyl, -cyclohexyl, -cyclohexenyl, -1,3-cyclohexadienyl, -1,4-cyclohexadienyl, -cycloheptyl, -1,3-cycloheptadienyl, -1,3,5-cycloheptatrienyl, -cyclooctyl, and -cyclooctadienyl. A C₃-C₈ carbocycle group can be unsubstituted or substituted with one or more groups including, but not limited to, —C₁₋₁₂alkyl, —O—(C₁₋₁₂alkyl), -aryl, —C(O)R′, —OC(O)R′, —C(O)OR′, —C(O)NH₂, —C(O)NHR′, —C(O)N(R′)₂—NHC(O)R′, —S(O)₂R′, —S(O)R′, —OH, -halogen, —N₃, —NH₂, —NH(R′), —N(R′)₂ and —CN; where each R′ is independently selected from H, —C₁₋₁₂ alkyl and aryl.

A “C₃₋₁₂ carbocyclo” refers to a C₃-C₈ carbocycle group defined above wherein one of the carbocycle groups' hydrogen atoms is replaced with a bond.

A “C₁₋₁₀alkylene” is a straight chain, saturated hydrocarbon group of the formula —(CH₂)₁₋₁₀—. Examples of a C₁-C₁₀ alkylene include methylene, ethylene, propylene, butylene, pentylene, hexylene, heptylene, ocytylene, nonylene and decalene.

An “arylene” is an aryl group which has two covalent bonds and can be in the ortho, meta, or para configurations as shown in the following structures:

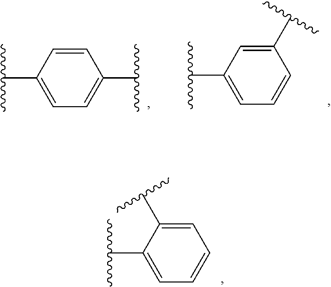 [SMILES: *c1ccc(*)cc1 ; *c1cccc(*)c1 ; *c1ccccc1*]

in which the phenyl group can be unsubstituted or substituted with up to four groups including, but not limited to, —C₁-C₈ alkyl, —O—(C₁-C₈ alkyl), -aryl, —C(O)R′, —OC(O)R′, —C(O)OR′, —C(O)NH₂, —C(O)NHR′, —C(O)N(R′)₂—NHC(O)R′, —S(O)₂R′, —S(O)R′, —OH, -halogen, —N₃, —NH₂, —NH(R′), —N(R′)₂ and —CN; wherein each R′ is independently selected from H, —C₁-C₈ alkyl and aryl.

A “C₃₋₁₂heterocyclyl” refers to an aromatic or non-aromatic C₃₋₁₂carbocycle in which one to four of the ring carbon atoms are independently replaced with a heteroatom from the group consisting of O, S and N. Representative examples of a C₃-C₈ heterocycle include, but are not limited to, benzofuranyl, benzothiophene, indolyl, benzopyrazolyl, coumarinyl, isoquinolinyl, pyrrolyl, thiophenyl, furanyl, thiazolyl, imidazolyl, pyrazolyl, triazolyl, quinolinyl, pyrimidinyl, pyridinyl, pyridonyl, pyrazinyl, pyridazinyl, isothiazolyl, isoxazolyl and tetrazolyl. A C₃-C₁₂ heterocycle can be unsubstituted or substituted with up to seven groups including, but not limited to, —C₁-C₈ alkyl, —O—(C₁-C₈ alkyl), -aryl, —C(O)R′, —OC(O)R′, —C(O)OR′, —C(O)NH₂, —C(O)NHR′, —C(O)N(R′)₂—NHC(O)R′, —S(O)₂R′, —S(O)R′, —OH, -halogen, —N₃, —NH₂, —NH(R′), —N(R′)₂ and —CN; wherein each R′ is independently selected from H, —C₁-C₈ alkyl and aryl.

“C₃₋₁₂heterocyclo” refers to a C₃₋₁₂heterocycle group defined above wherein one of the heterocycle group's hydrogen atoms is replaced with a bond. A C₃-C₁₂ heterocyclo can be unsubstituted or substituted with up to six groups including, but not limited to, —C₁-C₁₂alkyl, —O—(C₁-C₁₂ alkyl), -aryl, —C(O)R′, —OC(O)R′, —C(O)OR′, —C(O)NH₂, —C(O)NHR′, —C(O)N(R′)₂—NHC(O)R′, —S(O)₂R′, —S(O)R′, —OH, -halogen, —N₃, —NH₂, —NH(R′), —N(R′)₂ and —CN; wherein each R′ is independently selected from H, —C₁-C₁₂alkyl and aryl.

“Alkenylene” refers to an unsaturated, branched or straight chain or cyclic hydrocarbon radical of 2-18 carbon atoms, and having two monovalent radical centers derived by the removal of two hydrogen atoms from the same or two different carbon atoms of a parent alkene. Typical alkenylene radicals include, but are not limited to: 1,2-ethylene (—CH═CH—).

“Alkynylene” refers to an unsaturated, branched or straight chain or cyclic hydrocarbon radical of 2-18 carbon atoms, and having two monovalent radical centers derived by the removal of two hydrogen atoms from the same or two different carbon atoms of a parent alkyne. Typical alkynylene radicals include, but are not limited to: acetylene (—C≡C—), propargyl (—CH₂C≡C—), and 4-pentynyl (—CH₂CH₂CH₂C≡CH—).

“Arylalkyl” refers to an acyclic alkyl radical in which one of the hydrogen atoms bonded to a carbon atom, typically a terminal or sp³ carbon atom, is replaced with an aryl radical. Typical arylalkyl groups include, but are not limited to, benzyl, 2-phenylethan-1-yl, 2-phenylethen-1-yl, naphthylmethyl, 2-naphthylethan-1-yl, 2-naphthylethen-1-yl, naphthobenzyl, 2-naphthophenylethan-1-yl and the like. The arylalkyl group comprises 6 to 20 carbon atoms, e.g., the alkyl moiety, including alkanyl, alkenyl or alkynyl groups, of the arylalkyl group is 1 to 6 carbon atoms and the aryl moiety is 5 to 14 carbon atoms.

“Heteroarylalkyl” refers to an acyclic alkyl radical in which one of the hydrogen atoms bonded to a carbon atom, typically a terminal or sp³ carbon atom, is replaced with a heteroaryl radical. Typical heteroarylalkyl groups include, but are not limited to, 2-benzimidazolylmethyl, 2-furylethyl, and the like. The heteroarylalkyl group comprises 6 to 20 carbon atoms, e.g., the alkyl moiety, including alkanyl, alkenyl or alkynyl groups, of the heteroarylalkyl group is 1 to 6 carbon atoms and the heteroaryl moiety is 5 to 14 carbon atoms and 1 to 3 heteroatoms selected from N, O, P, and S. The heteroaryl moiety of the heteroarylalkyl group may be a monocycle having 3 to 7 ring members (2 to 6 carbon atoms or a bicycle having 7 to 10 ring members (4 to 9 carbon atoms and 1 to 3 heteroatoms selected from N, O, P, and S), for example: a bicyclo [4,5], [5,5], [5,6], or [6,6] system.

“Substituted alkyl”, “substituted aryl”, and “substituted arylalkyl” mean alkyl, aryl, and arylalkyl respectively, in which one or more hydrogen atoms are each independently replaced with a substituent. Typical substituents include, but are not limited to, —X, —R, —O⁻, —OR, —SR, —S⁻, —NR₂, —NR₃, ═NR, —CX₃, —CN, —OCN, —SCN, —N═C═O, —NCS, —NO, —NO₂, ═N₂, —N₃, NC(═O)R, —C(═O)R, —C(═O)NR₂, —SO₃⁻, —SO₃H, —S(═O)₂R, —OS(═O)₂OR, —S(═O)₂NR, —S(═O)R, —OP(═O)(OR)₂, —P(═O)(OR)₂, PO₃, —PO₃H₂, —C(═O)R, —C(═O)X, —C(═S)R, —CO₂R, —CO₂⁻, —C(═S)OR, —C(═O)SR, —C(═S)SR, —C(═O)NR₂, —C(═S)NR₂, —C(═NR)NR₂, where each X is independently a halogen: F, Cl, Br, or I; and each R is independently —H, C₂-C₂₀ alkyl, C₆-C₂₀ aryl, C₃-C₁₄ heterocycle, protecting group or prodrug moiety. Alkylene, alkenylene, and alkynylene groups as described above may also be similarly substituted.

Examples of heterocycles include by way of example and not limitation pyridyl, dihydroypyridyl, tetrahydropyridyl (piperidyl), thiazolyl, tetrahydrothiophenyl, sulfur oxidized tetrahydrothiophenyl, pyrimidinyl, furanyl, thienyl, pyrrolyl, pyrazolyl, imidazolyl, tetrazolyl, benzofuranyl, thianaphthalenyl, indolyl, indolenyl, quinolinyl, isoquinolinyl, benzimidazolyl, piperidinyl, 4-piperidonyl, pyrrolidinyl, 2-pyrrolidonyl, pyrrolinyl, tetrahydrofuranyl, bis-tetrahydrofuranyl, tetrahydropyranyl, bis-tetrahydropyranyl, tetrahydroquinolinyl, tetrahydroisoquinolinyl, decahydroquinolinyl, octahydroisoquinolinyl, azocinyl, triazinyl, 6H-1,2,5-thiadiazinyl, 2H,6H-1,5,2-dithiazinyl, thienyl, thianthrenyl, pyranyl, isobenzofuranyl, chromenyl, xanthenyl, phenoxathinyl, 2H-pyrrolyl, isothiazolyl, isoxazolyl, pyrazinyl, pyridazinyl, indolizinyl, isoindolyl, 3H-indolyl, 1H-indazolyl, purinyl, 4H-quinolizinyl, phthalazinyl, naphthyridinyl, quinoxalinyl, quinazolinyl, cinnolinyl, pteridinyl, 4aH-carbazolyl, carbazolyl, β-carbolinyl, phenanthridinyl, acridinyl, pyrimidinyl, phenanthrolinyl, phenazinyl, phenothiazinyl, furazanyl, phenoxazinyl, isochromanyl, chromanyl, imidazolidinyl, imidazolinyl, pyrazolidinyl, pyrazolinyl, piperazinyl, indolinyl, isoindolinyl, quinuclidinyl, morpholinyl, oxazolidinyl, benzotriazolyl, benzisoxazolyl, oxindolyl, benzoxazolinyl, and isatinoyl.

By way of example and not limitation, carbon bonded heterocycles are bonded at position 2, 3, 4, 5, or 6 of a pyridine, position 3, 4, 5, or 6 of a pyridazine, position 2, 4, 5, or 6 of a pyrimidine, position 2, 3, 5, or 6 of a pyrazine, position 2, 3, 4, or 5 of a furan, tetrahydrofuran, thiofuran, thiophene, pyrrole or tetrahydropyrrole, position 2, 4, or 5 of an oxazole, imidazole or thiazole, position 3, 4, or 5 of an isoxazole, pyrazole, or isothiazole, position 2 or 3 of an aziridine, position 2, 3, or 4 of an azetidine, position 2, 3, 4, 5, 6, 7, or 8 of a quinoline or position 1, 3, 4, 5, 6, 7, or 8 of an isoquinoline. Still more typically, carbon bonded heterocycles include 2-pyridyl, 3-pyridyl, 4-pyridyl, 5-pyridyl, 6-pyridyl, 3-pyridazinyl, 4-pyridazinyl, 5-pyridazinyl, 6-pyridazinyl, 2-pyrimidinyl, 4-pyrimidinyl, 5-pyrimidinyl, 6-pyrimidinyl, 2-pyrazinyl, 3-pyrazinyl, 5-pyrazinyl, 6-pyrazinyl, 2-thiazolyl, 4-thiazolyl, or 5-thiazolyl.

By way of example and not limitation, nitrogen bonded heterocycles are bonded at position 1 of an aziridine, azetidine, pyrrole, pyrrolidine, 2-pyrroline, 3-pyrroline, imidazole, imidazolidine, 2-imidazoline, 3-imidazoline, pyrazole, pyrazoline, 2-pyrazoline, 3-pyrazoline, piperidine, piperazine, indole, indoline, 1H-indazole, position 2 of a isoindole, or isoindoline, position 4 of a morpholine, and position 9 of a carbazole, or β-carboline. Still more typically, nitrogen bonded heterocycles include 1-aziridyl, 1-azetedyl, 1-pyrrolyl, 1-imidazolyl, 1-pyrazolyl, and 1-piperidinyl.

### Polynucleotides

In some embodiments the cGAS-STING pathway inhibitor is a polynucleotide, which may inhibit cGAS-STING mediated signalling activity by at least one of a number of different mechanisms.

RNA Interference

In some embodiments the polynucleotide cGAS-STING pathway inhibitor acts by reducing expression of cGAS and/or STING proteins by targeting their mRNA. For example, the polynucleotide may reduce expression of cGAS or STING by RNA interference.

The terms “RNA interference”, “RNAi” or “gene silencing” refer generally to a process in which a double-stranded RNA molecule reduces the expression of a nucleic acid sequence with which the double-stranded RNA molecule shares substantial or total homology. However, it has been shown that RNA interference can also be achieved using non-RNA double stranded molecules (see, for example, US 20070004667).

In some embodiments, a cGAS-STING pathway inhibitor comprises nucleic acid molecules comprising and/or encoding double-stranded regions for RNA interference against cGAS mRNA or STING mRNA. The nucleic acid molecules are typically RNA, but may comprise chemically-modified nucleotides and non-nucleotides.

The double-stranded regions should be at least 19 contiguous nucleotides, for example about 19 to 23 nucleotides, or may be longer, for example 30 or 50 nucleotides, or 100 nucleotides or more. The full-length sequence corresponding to the entire gene transcript may be used. Preferably, they are about 19 to about 23 nucleotides in length.

The degree of identity of a double-stranded region of a nucleic acid molecule to the targeted transcript should be at least 90% and more preferably 95-100%. The nucleic acid molecule may of course comprise unrelated sequences which may function to stabilize the molecule.

The term “short interfering RNA” or “siRNA” as used herein refers to a nucleic acid molecule which comprises ribonucleotides capable of inhibiting or down regulating gene expression, for example by mediating RNAi in a sequence-specific manner, wherein the double stranded portion is less than 50 nucleotides in length, preferably about 19 to about 23 nucleotides in length. For example, the siRNA can be a nucleic acid molecule comprising self-complementary sense and antisense regions, wherein the antisense region comprises nucleotide sequence that is complementary to nucleotide sequence in a target nucleic acid molecule or a portion thereof and the sense region having nucleotide sequence corresponding to the target nucleic acid sequence or a portion thereof. The siRNA can be assembled from two separate oligonucleotides, where one strand is the sense strand and the other is the antisense strand, wherein the antisense and sense strands are self-complementary.

As used herein, the term siRNA is meant to be equivalent to other terms used to describe nucleic acid molecules that are capable of mediating sequence specific RNAi, for example micro-RNA (miRNA), short hairpin RNA (shRNA), short interfering oligonucleotide, short interfering nucleic acid (siNA), short interfering modified oligonucleotide, chemically-modified siRNA, post-transcriptional gene silencing RNA (ptgsRNA), and others. In addition, as used herein, the term RNAi is meant to be equivalent to other terms used to describe sequence specific RNA interference, such as post transcriptional gene silencing, translational inhibition, or epigenetics. For example, siRNA molecules can be used to epigenetically silence genes at both the post-transcriptional level or the pre-transcriptional level. In a non-limiting example, epigenetic regulation of gene expression by siRNA molecules can result from siRNA mediated modification of chromatin structure to alter gene expression.

By “shRNA” or “short-hairpin RNA” is meant an RNA molecule where less than about 50 nucleotides, preferably about 19 to about 23 nucleotides, is base paired with a complementary sequence located on the same RNA molecule, and where said sequence and complementary sequence are separated by an unpaired region of at least about 4 to about 15 nucleotides which forms a single-stranded loop above the stem structure created by the two regions of base complementarity.

Included shRNAs are dual or bi-finger and multi-finger hairpin dsRNAs, in which the RNA molecule comprises two or more of such stem-loop structures separated by single-stranded spacer regions.

Once designed, the nucleic acid molecules comprising a double-stranded region can be generated by any method known in the art, for example, by in vitro transcription, recombinantly, or by synthetic means.

Modifications or analogs of nucleotides can be introduced to improve the properties of the nucleic acid molecules. Improved properties include increased nuclease resistance and/or increased ability to permeate cell membranes. Accordingly, the terms “nucleic acid molecule” and “double-stranded RNA molecule” includes synthetically modified bases such as, but not limited to, inosine, xanthine, hypoxanthine, 2-aminoadenine, 6-methyl-, 2-propyl- and other alkyl-adenines, 5-halo uracil, 5-halo cytosine, 6-aza cytosine and 6-aza thymine, pseudo uracil, 4-thiuracil, 8-halo adenine, 8-aminoadenine, 8-thiol adenine, 8-thiolalkyl adenines, 8-hydroxyl adenine and other 8-substituted adenines, 8-halo guanines, 8-amino guanine, 8-thiol guanine, 8-thioalkyl guanines, 8-hydroxyl guanine and other substituted guanines, other aza and deaza adenines, other aza and deaza guanines, 5-trifluoromethyl uracil and 5-trifluoro cytosine.

Chemically modified siRNAs particularly suited for in vivo delivery are described in the art in, e.g., WO2014201306, WO2007051303.

Polynucleotides Encoding Peptides or Polypeptides

In some embodiments, a polynucleotide-based cGAS-STING pathway inhibitor encodes a polypeptide, so that delivery of the polynucleotide to cells results in expression of an encoded peptide or polypeptide cGAS-STING pathway protein inhibitor.

In some embodiments, the polynucleotide encodes a dominant negative suppressor of cGAS-STING mediated signalling activity.

In some embodiments, the polynucleotide cGAS-STING pathway inhibitor encodes a programmable nuclease which inhibits cGAS-STING mediated signalling activity by inactivating or reducing expression of the genes encoding cGAS or STING. As used herein, the term “programmable nuclease” relates to nucleases that are “targeted” (“programmed”) to recognize and edit a pre-determined genomic location. In some embodiments the encoded polypeptide is a programmable nuclease “targeted” or “programmed” to introduce a genetic modification into the cGAS or STING encoding gene or regulatory region thereof. In some embodiments, the genetic modification is a deletion or substitution in the cGAS or STING encoding gene or in a regulatory region thereof.

In some embodiments, the programmable nuclease may be programmed to recognize a genomic location by a combination of DNA-binding zinc-finger protein (ZFP) domains. ZFPs recognize a specific 3-bp in a DNA sequence, a combination of ZFPs can be used to recognize a specific a specific genomic location. In some embodiments, the programmable nuclease may be programmed to recognize a genomic location by transcription activator-like effectors (TALEs) DNA binding domains. In an alternate embodiment, the programmable nuclease may be programmed to recognize a genomic location by one or more RNA sequences. In an alternate embodiment, the programmable nuclease may be programmed by one or more DNA sequences. In an alternate embodiment, the programmable nuclease may be programmed by one or more hybrid DNA/RNA sequences. In an alternate embodiment, the programmable nuclease may be programmed by one or more of an RNA sequence, a DNA sequences and a hybrid DNA/RNA sequence.

Programmable nucleases that can be used in accordance with the present disclosure include, but are not limited to, RNA-guided engineered nuclease (RGEN) derived from the bacterial clustered regularly interspaced short palindromic repeat (CRISPR)-cas (CRISPR-associated) system, zinc-finger nuclease (ZFN), transcription activator-like nuclease (TALEN), and argonautes.

In some embodiments, the nuclease is a RNA-guided engineered nuclease (RGEN). In some embodiments the RGEN is from an archaeal genome or is a recombinant version thereof. In some embodiments the RGEN is from a bacterial genome or is a recombinant version thereof. In some embodiments the RGEN is from a Type I (CRISPR)-cas (CRISPR-associated) system. In some embodiments the RGEN is from a Type II (CRISPR)-cas (CRISPR-associated) system. In some embodiments the RGEN is from a Type III (CRISPR)-cas (CRISPR-associated) system. In some embodiments the nuclease is a class I RGEN. In some embodiments the nuclease is a class II RGEN. In some embodiments the RGEN is a multi-component enzyme. In some embodiments the RGEN is a single component enzyme. In some embodiments the RGEN is CAS3. In some embodiments, the RGEN is CASIO. In some embodiments the RGEN is CAS9. In some embodiments, the RGEN is Cpf1 (Zetsche et al., 2015). In some embodiments, the RGEN is targeted by a single RNA or DNA. In some embodiments, the RGEN is targeted by more than one RNA and/or DNA. In some embodiments, the programmable nuclease may be a DNA programmed argonaute (WO 14/189628).

In some embodiments, the polynucleotide cGAS-STING pathway inhibitor is provided in an expression vector to be delivered to cells (e.g., neurons) using any of a number of routine targeting methods known in the art.

As used herein, an “expression vector” is a DNA or RNA vector that is capable of effecting expression of one or more polynucleotides in a host cell (e.g., a neuron). The vector is typically a plasmid or recombinant virus. Any suitable expression vector can be used, examples of which include, but are not limited to, a plasmid or viral vector. In some embodiments, the viral vector is a retrovirus, a lentivirus, an adenovirus, a herpes virus, or an adeno-associated viral vector.

Such vectors will include one or more promoters for expressing the polynucleotide such as a dsRNA for gene silencing. Suitable promoters include include, but are not limited to, the retroviral LTR; the SV40 promoter; and the human cytomegalovirus (CMV) promoter. Cellular promoters such as eukaryotic cellular promoters including, but not limited to, the histone, RNA polymerase III (in the case of shRNA or miRNA expression), and β-actin promoters, can also be used. In some embodiments the promoter is an NK cell-selective promoter such as the human NKp46 promoter (see, e.g., Walzer et al., 2007). The selection of a suitable promoter will be apparent to those skilled in the art from the teachings contained herein.

### Polypeptides

In some embodiments the cGAS-STING pathway inhibitor is a polypeptide, which may inhibit cGAS-STING signalling activity by at least one of a number of different mechanisms, e.g., specifically binding to cGAS, STING, cGAMP or TBK-1 thereby reducing interaction of these molecules with their ligands/binding partners.

Polypeptides Comprising Antigen Binding Sites

In some embodiments, a cGAS-STING pathway inhibitor is a polypeptide comprising an antigen binding site such as an antibody or a fragment thereof. Preferably, the antibody or fragment thereof is modified to penetrate or be taken up (passively or actively) in mammalian cells, and particularly neurons.

The term “antibody” as used herein includes polyclonal antibodies, monoclonal antibodies, bispecific antibodies, fusion diabodies, triabodies, heteroconjugate antibodies, and chimeric antibodies. Also contemplated are antibody fragments that retain at least substantial (about 10%) antigen binding relative to the corresponding full length antibody. Such antibody fragments are referred to herein as “antigen-binding fragments” and comprise an antigen binding site of an antibody. Antibodies include modifications in a variety of forms including, for example, but not limited to, domain antibodies including either the VH or VL domain, a dimer of the heavy chain variable region (VHH, as described for a camelid), a dimer of the light chain variable region (VLL), Fv fragments containing only the light (VL) and heavy chain (VH) variable regions which may be joined directly or through a linker, or Fd fragments containing the heavy chain variable region and the CHI domain.

A scFv consisting of the variable regions of the heavy and light chains linked together to form a single-chain antibody and oligomers of scFvs such as diabodies and triabodies are also encompassed by the term “antibody”. Also encompassed are fragments of antibodies such as Fab, (Fab′)2 and FabFc2 fragments which contain the variable regions and parts of the constant regions. Complementarity determining region (CDR)-grafted antibody fragments and oligomers of antibody fragments are also encompassed. The heavy and light chain components of an Fv may be derived from the same antibody or different antibodies thereby producing a chimeric Fv region. The antibody may be of animal (for example mouse, rabbit or rat) or human origin or may be chimeric or humanize.

As used herein the term “antibody” includes these various forms. Using the guidelines provided herein and those methods well known to those skilled in the art which are described in the references cited above and in such publications as Harlow & Lane, Antibodies: a Laboratory Manual, Cold Spring Harbor Laboratory, (1988) the antibodies for use in the methods of the present invention can be readily made.

The antibodies may be Fv regions comprising a variable light (VL) and a variable heavy (VH) chain in which the light and heavy chains may be joined directly or through a linker. As used herein a linker refers to a molecule that is covalently linked to the light and heavy chain and provides enough spacing and flexibility between the two chains such that they are able to achieve a conformation in which they are capable of specifically binding the epitope to which they are directed. Protein linkers are particularly preferred as they may be expressed as an intrinsic component of the Ig portion of the fusion polypeptide.

In another embodiment, recombinantly produced single chain scFv antibody, preferably a humanized scFv, is used in the methods of the invention.

In one embodiment, the antibodies have the capacity for intracellular transmission. Antibodies which have the capacity for intracellular transmission include antibodies such as camelids and llama antibodies, shark antibodies (IgNARs), scFv antibodies, intrabodies or nanobodies, for example, scFv intrabodies and VHH intrabodies. Yeast SPLINT antibody libraries are available for testing for intrabodies which are able to disrupt protein-protein interactions. Such agents may comprise a cell-penetrating peptide sequence or nuclear-localizing peptide sequence such as those disclosed in Constantini et al. (2008). Also useful for in vivo delivery are Vectocell or Diato peptide vectors such as those disclosed in De Coupade et al. (2005).

In addition, the antibodies may be fused to a cell penetrating agent, for example a cell-penetrating peptide. Cell penetrating peptides include Tat peptides, Penetratin, short amphipathic peptides such as those from the Pep- and MPG-families, oligoarginine and oligolysine. In one example, the cell penetrating peptide is also conjugated to a lipid (C6-C18 fatty acid) domain to improve intracellular delivery (Koppelhus et al., 2008). Examples of cell penetrating peptides can be found in Howl et al. (2007) and Deshayes et al. (2008). Thus, the invention also provides the therapeutic use of antibodies fused via a covalent bond (e.g. a peptide bond), at optionally the N-terminus or the C-terminus, to a cell-penetrating peptide sequence.

Methods for generating antibodies and fragments thereof are known in the art and/or described in Harlow and Lane (editors) Antibodies: A Laboratory Manual, Cold Spring Harbor Laboratory, (1988). Generally, in such methods, an antigen or a region thereof (e.g., an extracellular domain) or immunogenic fragment or epitope thereof or a cell expressing and displaying same (i.e., an immunogen), optionally formulated with any suitable or desired carrier, adjuvant, or pharmaceutically acceptable excipient, is administered to a non-human animal, for example, a mouse, chicken, rat, rabbit, guinea pig, dog, horse, cow, goat or pig. The immunogen may be administered intranasally, intramuscularly, sub-cutaneously, intravenously, intradermally, intraperitoneally, or by other known route.

Monoclonal antibodies are one exemplary form of an antibody contemplated by the present disclosure. The term “monoclonal antibody” or “mAb” refers to a homogeneous antibody population capable of binding to the same antigen(s), for example, to the same epitope within the antigen. This term is not intended to be limited as regards to the source of the antibody or the manner in which it is made.

For the production of mAbs any one of a number of known techniques may be used, such as, for example, the procedure exemplified in U.S. Pat. No. 4,196,265.

Alternatively, ABL-MYC technology (NeoClone, Madison Wis. 53713, USA) is used to produce cell lines secreting MAbs (e.g., as described in Largaespada et al., 1996).

Antibodies can also be produced or isolated by screening a display library, e.g., a phage display library, e.g., as described in U.S. Pat. No. 6,300,064 and/or U.S. Pat. No. 5,885,793.

The antibody of the present disclosure may be a synthetic antibody. For example, the antibody is a chimeric antibody, a humanized antibody, a human antibody or a de-immunized antibody.

In one embodiment, an antibody described herein is a chimeric antibody. The term “chimeric antibody” refers to antibodies in which a portion of the heavy and/or light chain is identical with or homologous to corresponding sequences in antibodies derived from a particular species (e.g., murine, such as mouse) or belonging to a particular antibody class or subclass, while the remainder of the chain(s) is identical with or homologous to corresponding sequences in antibodies derived from another species (e.g., primate, such as human) or belonging to another antibody class or subclass. Methods for producing chimeric antibodies are described in, e.g., U.S. Pat. Nos. 4,816,567 and 5,807,715.

The antibodies of the present disclosure may be humanized or human.

The term “humanized antibody” shall be understood to refer to a subclass of chimeric antibodies having an antigen binding site or variable region derived from an antibody from a non-human species and the remaining antibody structure based upon the structure and/or sequence of a human antibody. In a humanized antibody, the antigen-binding site generally comprises the complementarity determining regions (CDRs) from the non-human antibody grafted onto appropriate FRs in the variable regions of a human antibody and the remaining regions from a human antibody. Antigen binding sites may be wild-type (i.e., identical to those of the non-human antibody) or modified by one or more amino acid substitutions. In some instances, FR residues of the human antibody are replaced by corresponding non-human residues.

Methods for humanizing non-human antibodies or parts thereof (e.g., variable regions) are known in the art. Humanization can be performed following the method of U.S. Pat. No. 5,225,539 or 5,585,089. Other methods for humanizing an antibody are not excluded.

STING Inhibitors

In some embodiments, the cGAS-STING pathway inhibitor is a STING inhibitor. Such inhibitors can specifically target STING by, for example, reducing STING activity (e.g., TPK1 activation), competitively inhibiting binding of STING to cGAMP, or reducing STING expression.

In some embodiments, the STING inhibitor is a cyclic dinucleotide. Such cyclic dinucleotide inhibitors are believed to occupy the cGAMP binding site on STING while preventing STING from activating TPK1. Suitable cyclic dinucleotides are described in US20140341976 and WO2015185565. In some embodiments, the cyclic dinucleotide is a compound of formula (I)

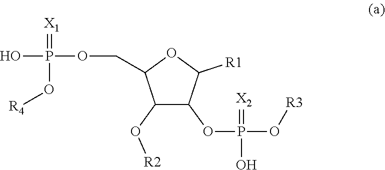

covalently linked to

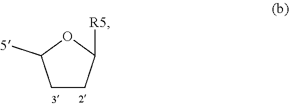

wherein the following definitions are provided in relation to the substitution of the compound of formula (I):
R3 is a covalent bond to the 5′ carbon of (b),
R4 is a covalent bond to the 2′ or 3′ carbon of (b),
R1 is a purine linked through its N9 nitrogen to the ribose ring of (a),
R5 is a purine linked through its N9 nitrogen to the ribose ring of (b),
Each of X1 and X2 are independently O or S,
R2 is H or an optionally substituted straight chain alkyl of from 1 to 18 carbons and from 0 to 3 heteroatoms, an optionally substituted alkenyl of from 1-9 carbons, an optionally substituted alkynyl of from 1-9 carbons, or an optionally substituted aryl, wherein substitution(s), when present, may be independently selected from the group consisting of C1-6 alkyl straight or branched chain, benzyl, halogen, trihalomethyl, C1-6 alkoxy, —NO2, —NH2, —OH, ═O, —COOR′ where R′ is H or lower alkyl, —CH2OH, and —CONH2, and
the 2′ or 3′ carbon of (b) which is not in a covalent bond with (a) is —O—R6, wherein R6 is H or an optionally substituted straight chain alkyl of from 1 to 18 carbons and from 0 to 3 heteroatoms, an optionally substituted alkenyl of from 1-9 carbons, an optionally substituted alkynyl of from 1-9 carbons, or an optionally substituted aryl, wherein substitution(s), when present, may be independently selected from the group consisting of C1-6 alkyl straight or branched chain, benzyl, halogen, trihalomethyl, C1-6 alkoxy, —NO2, —NH2, —OH, ═O, —COOR′ where R′ is H or lower alkyl, —CH2OH, and —CONH2,
or a pharmaceutically acceptable salt, solvate, stereoisomer, tautomer, or prodrug thereof.

In some embodiments, R2 and R6 are not both H. In some embodiments one or both of R2 and R6 are independently an unsubstituted straight chain alkyl of from 1 to 18 carbons, an unsubstituted alkenyl of from 1-9 carbons, an unsubstituted alkynyl of from 1-9 carbons, or an unsubstituted aryl, and most preferably selected from the group consisting of selected from the group consisting of allyl, propargyl, homoallyl, homopropargyl, methyl, ethyl, propyl, isopropyl, isobutyl, cyclopropylmethyl, and benzyl. In some embodiments, one or both of R2 and R6 are allyl. In some embodiments, one or both of R2 and R6 comprise a propargyl. In some embodiments, one or both of R2 and R6 are methyl. In some embodiments, one or both of R2 and R6 are ethyl. In some embodiments, one or both of R2 and R6 are propyl. In some embodiments, one or both of R2 and R6 are benzyl. In some embodiments, one of R2 or R6 is selected from the group consisting of allyl, propargyl, homoallyl, homopropargyl, methyl, ethyl, propyl, isopropyl, isobutyl, cyclopropylmethyl, and benzyl, and the other of R2 or R6 comprises a prodrug leaving group.

In some embodiments, the prodrug leaving group is a moiety removed by cellular esterases. In some embodiments, the prodrug leaving group is a C6 to C18 fatty acid ester.

In some embodiments, X1 and X2 are both S.

In some embodiments, R1 and R5 are independently selected from the group consisting of adenine, guanine, inosine, and xanthine. In some embodiments, one or both of R1 and R5 are adenine. In some embodiments, one or both of R1 and R5 are guanine. In some embodiments, R1 is adenine and R5 is guanine.

Other STING inhibitors encompassed by the present disclosure include the small molecule compounds described in Haag et al. (2018). Such compounds covalently target the predicted transmembrane residue Cys91 and thereby block activation-induced palmitoylation of STING. It is believed that palmitoylation of STING is essential for its assembly into multimeric complexes at the Golgi apparatus and, in turn, for the recruitment of downstream signalling factors. Thus, STING inhibitors and their derivatives that target Cys91 are predicted to reduce STING mediated inflammatory cytokine production. Thus, in some embodiments, a STING inhibitor for use in the present invention blocks palmitoylation-induced clustering of STING. In some embodiments, the STING inhibitor covalently binds to STING. In some embodiments, the STING inhibitor covalently binds to Cys91 of STING. In some embodiments, the STING inhibitor is a nitrofuran derivative. In some embodiments, the STING inhibitor is H-151 as described in Haag et al. (2018).

In one embodiment, the STING inhibitor may be a compound of formula (II) or a pharmaceutically acceptable salt, solvate, stereoisomer, tautomer, or prodrug thereof:

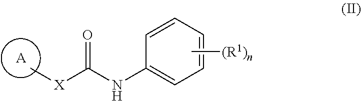

wherein the following definitions are provided in relation to the substitution of the compound of formula (II), including formula (IIa), (IIb), (IIc), (IId), (IId), (IIe), (IIf):

n represents 0 to 5;

Ring A is an heterocyclic or carbocyclic ring, wherein the heterocyclic and carbocyclic is optionally substituted with halo, CN, NO₂, OC(O)R², C(O)R², C(O)NR²R³, C(O)OR², OR², OS(O)₂R², NR²R³, SR², and R²; wherein R² and R³ are each independently selected from hydrogen, C₁₋₁₀alkyl, C₁₋₁₀alkylhalo, arylC₁₋₁₀alkyl, hetarylC₁₋₁₀alkyl, and heterocyclic;

X is either absent, or selected from the group consisting of NR², C₁₋₁₀alkyl, C₁₋₁₀alkenyl, C₂₋₁₀ alkynyl, wherein the C₁₋₁₀alkyl, C₂₋₁₀alkenyl, C₂₋₁₀alkynyl, are each optionally interrupted with one or more heteroatoms independently selected from O, N and S, wherein C₁₋₁₀alkyl, C₂₋₁₀alkenyl, C₂₋₁₀ alkynyl are each optionally substituted with halo, CN, NO₂, OC(O)R², C(O)R², C(O)NR²R³, C(O)OR², OR², OS(O)₂R², NR²R³, SR², and R²; wherein R² and R³ may be provided according to any embodiments as described herein; and

R¹ is independently selected from hydrogen, halo, CN, NO₂, OC(O)R⁴, C(O)R⁴, C(O)NR⁴R⁵, C(O)OR⁴, OR⁴, OS(O)₂R⁴, NR⁴R⁵, SR⁴ and R⁴, wherein R⁴ and R⁵ are each independently selected from hydrogen, C₁₋₁₀alkyl, C₁₋₁₀alkylhalo, arylC₁₋₁₀alkyl, hetarylC₁₋₁₀alkyl, and heterocyclic, wherein the C₁₋₁₀alkyl moiety is optionally interrupted with one or more heteroatoms independently selected from O, N and S, and the C₁₋₁₀alkyl, arylC₁₋₁₀alkyl, hetarylC₁₋₁₀alkyl, and heterocyclic groups are each optionally substituted with halo, CN, NO₂, OC(O)R², C(O)R², C(O)NR²R³, C(O)OR², OR², OS(O)₂R², NR²R³, SR², and R²; wherein R² and R³ may be provided according to any embodiments as described herein.

In some embodiments, the STING inhibitor may be a compound of Formula (IIa) or Formula (IIb):

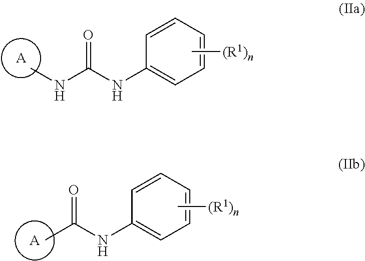

wherein n, Ring A and R¹, may be provided according to any embodiments as described herein according to formula (II).

In some embodiments, R¹ is independently selected from the group consisting of hydrogen, halo, C₁₋₁₀alkyl, C₁₋₁₀alkylhalo, arylC₁₋₁₀alkyl, hetarylC₁₋₁₀alkyl, heterocyclic, CN, and NO₂. In some embodiments, R¹ may be independently selected from hydrogen, halo and C₁₋₁₀alkyl. In one embodiments, R¹ is C₁₋₁₀alkyl, for example, methyl, ethyl, propyl, butyl, pentyl, hexyl, heptyl, octyl, nonyl or decyl. In one embodiment, R¹ is methyl, ethyl, propyl, butyl, pentyl, or hexyl. In another embodiment, R¹ is halo, for example I or Br. In another embodiment, R¹ is C₁₋₁₀alkylhalo, for example CF₃.

In some embodiments, Ring A may be a heterocyclic, for example a monocyclic or polycyclic heterocyclic, wherein the monocyclic or polycyclic heterocyclic may be an optionally substituted fully or partially saturated heterocyclic. The polycyclic heterocyclic may be a 5 or 6 membered heterocyclic ring which may be optionally fused with an optionally substituted monocyclic carbocyclic group. For example, Ring A may be a heterocyclic selected from the group consisting of pyridyl, dihydroypyridyl, tetrahydropyridyl (piperidyl), thiazolyl, tetrahydrothiophenyl, sulfur oxidized tetrahydrothiophenyl, pyrimidinyl, furanyl, thienyl, pyrrolyl, pyrazolyl, imidazolyl, tetrazolyl, benzofuranyl, thianaphthalenyl, indolyl, indolenyl, quinolinyl, isoquinolinyl, benzimidazolyl, piperidinyl, 4-piperidonyl, pyrrolidinyl, 2-pyrrolidonyl, pyrrolinyl, tetrahydrofuranyl, bis-tetrahydrofuranyl, tetrahydropyranyl, bis-tetrahydropyranyl, tetrahydroquinolinyl, tetrahydroisoquinolinyl, decahydroquinolinyl, octahydroisoquinolinyl, azocinyl, triazinyl, 6H-1,2,5-thiadiazinyl, 2H,6H-1,5,2-dithiazinyl, thienyl, thianthrenyl, pyranyl, isobenzofuranyl, chromenyl, xanthenyl, phenoxathinyl, 2H-pyrrolyl, isothiazolyl, isoxazolyl, pyrazinyl, pyridazinyl, indolizinyl, isoindolyl, 3H-indolyl, 1H-indazolyl, purinyl, 4H-quinolizinyl, phthalazinyl, naphthyridinyl, quinoxalinyl, quinazolinyl, cinnolinyl, pteridinyl, 4aH-carbazolyl, carbazolyl, β-carbolinyl, phenanthridinyl, acridinyl, pyrimidinyl, phenanthrolinyl, phenazinyl, phenothiazinyl, furazanyl, phenoxazinyl, isochromanyl, chromanyl, imidazolidinyl, imidazolinyl, pyrazolidinyl, pyrazolinyl, piperazinyl, indolinyl, isoindolinyl, quinuclidinyl, morpholinyl, oxazolidinyl, benzotriazolyl, benzisoxazolyl, oxindolyl, benzoxazolinyl, and isatinoyl, wherein the heterocyclic ring may be optionally substituted with hydrogen, halo, CN, NO₂, OC(O)R⁴, C(O)R⁴, C(O)NR⁴R⁵, C(O)OR⁴, OR⁴, OS(O)₂R⁴, NR⁴R⁵, SR⁴ and R⁴ wherein R⁴ and R⁵ may be provided according to any embodiments as described herein according to formula (II).

In some embodiments, Ring A may be a heterocyclic selected from a group of Formula (IIIa) of Formula (IIIb):

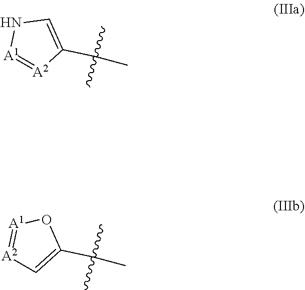

wherein the following definitions are provided in relation to the substitution of the compound of formula (IIIa) and (IIIb):

A¹ and A² may be each independently selected from CR⁶, or A¹ and A² may join together to form a carbocyclic or heterocyclic ring;

R⁶ may be independently selected from hydrogen, halo, hydrogen, halo, CN, NO₂, OC(O)R⁴, C(O)R⁴, C(O)NR⁴R⁵, C(O)OR⁴, OR⁴, OS(O)₂R⁴, NR⁴R⁵, SR⁴, wherein R⁴ and R⁵ may be provided according to any embodiments as described herein according to Formula (II);

wherein the C₁₋₁₀alkyl moiety, carbocyclic and heterocyclic may be each optionally substituted with one or more substituents independently selected from halo, CN, NO₂, OC(O)R⁴, C(O)R⁴, C(O)NR⁴R⁵, C(O)OR⁴, OR⁵, OS(O)₂R⁴, NR⁴R⁵, SR⁴, and R⁴; wherein R⁴ and R⁵ may be provided according to any embodiments as described herein according to Formula (II).

In one embodiment, Ring A may be selected from a group of Formula (IIIc)

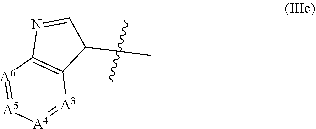

wherein the following definitions are provided in relation to the substitution of the compound of formula (IIIc):

A³, A⁴, A⁵ and A⁶, may be each independently selected from N and CR⁶

R⁶ may be selected from hydrogen, halo, hydrogen, halo, CN, NO₂, OC(O)R⁴, C(O)R⁴, C(O)NR⁴R⁵, C(O)OR⁴, OR⁴, OS(O)₂R⁴, NR⁴R⁵, SR⁴, wherein R⁴ and R⁵ may be provided according to any embodiments as described herein according to Formula (II).

In one embodiment, Ring A is indolyl or furanyl, wherein the indonyl or furanyl is optionally substituted with hydrogen, halo, CN, NO₂, OC(O)R⁴, C(O)R⁴, C(O)NR⁴R⁵, C(O)OR⁴, OR⁴, OS(O)₂R⁴, NR⁴R⁵, SR⁴, wherein R⁴ and R⁵ may be provided according to any embodiments as described herein according to Formula (II). In one embodiment, Ring A is indolyl optionally substituted with one or more C₁₋₆alkyl. In another embodiment, Ring A is furanyl optionally substituted with NO₂.

In some embodiments, the STING inhibitor may be a compound of Formula (lie) or Formula (IId):

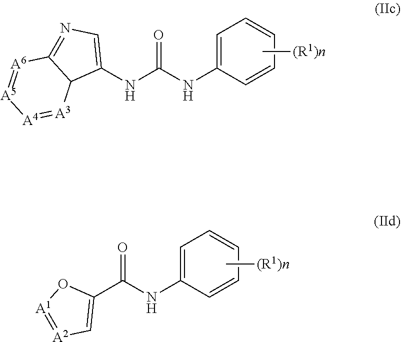

wherein A₁ to A₆, R₁ and n, may be provided according to any embodiments as described herein according to any one of Formula (II), (IIa), (IIb), (IIIa), (IIIb), and (IIIc).

In some embodiments, the STING inhibitor may be a compound of Formula (IIe) or Formula (IIf):

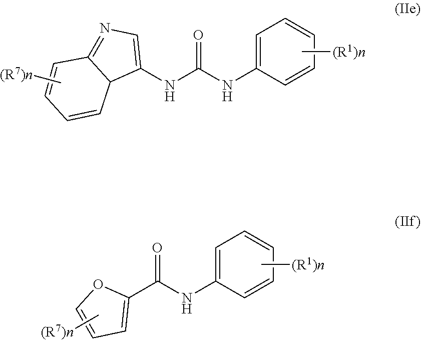

wherein

R¹, and n, may be provided according to any embodiments as described herein according to any one of Formula (II), (IIa), and (IIb);

R⁷ may be independently selected from hydrogen, halo, hydrogen, halo, CN, NO₂, OC(O)R⁴, C(O)R⁴, C(O)NR⁴R⁵, C(O)OR⁴, OR⁴, OS(O)₂R⁴, NR⁴R⁵, SR⁴, wherein R⁴ and R⁵ may be provided according to any embodiments as described herein.

In one embodiment, the STING inhibitor may be a compound selected from any one of the following compounds:

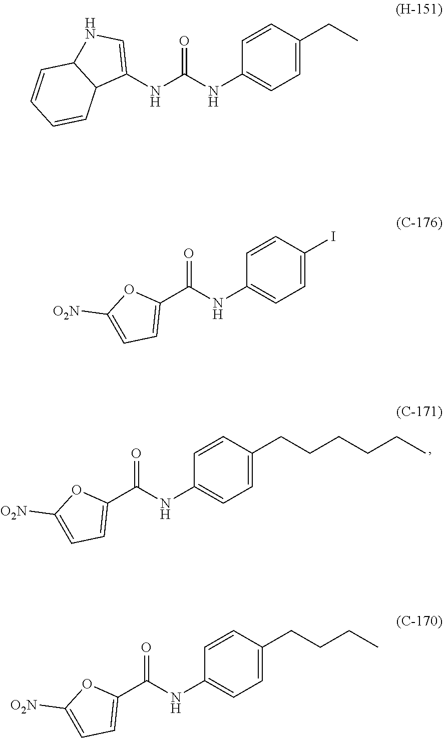 [SMILES: CCc1ccc(NC(=O)NC2=CNC3C=CC=CC23)cc1 ; O=C(Nc1ccc(I)cc1)c1ccc([N+](=O)[O-])o1 ; CCCCCCc1ccc(NC(=O)c2ccc([N+](=O)[O-])o2)cc1 ; CCCCc1ccc(NC(=O)c2ccc([N+](=O)[O-])o2)cc1]

In one preferred embodiment, the STING inhibitor is H-151.

In some embodiments, the STING inhibitor is a compound described in WO2013/166000. Such STING inhibitors include diclofenac, R(−)-2,10,11-trihydroxyaporphine hydrobromide, dipropyldopamine hydrobromide, (±)-trans-U-50488, 2,2′-bipyridine, SP600125, doxazosin mesylate, mitoxantrone, MRS 2159, nemadipine-A, (±)-PPHT hydrochloride, SMER28, quinine, or quisqualic acid. Such compounds are commercially available.

In some embodiments the STING inhibitor is a polynucleotide. For instance, the polynucleotide may inhibit STING by reducing the expression of STING. In some embodiments, the polynucleotide reduces the expression of STING by RNA interference. In some embodiments, the polynucleotide is an siRNA. Exemplary siRNAs that can be used to target STING mRNA are commercially available, e.g., from ThermoFisher (siRNA IDs: s50644, s50645, s50646, and S226307).

In some embodiments, the STING inhibitor is a polypeptide. In some embodiments, the polypeptide comprises an antigen binding site of an antibody which binds to STING. In some embodiments, the STING inhibitor is an antibody or a fragment thereof. Antibodies that target STING are commercially available from various sources such as R&D Systems (e.g., Cat. No. MAB7169). Such antibodies can be used to derive other polypeptides comprising antigen binding site using routine methods, such as, for example scFv antibodies, intrabodies or nanobodies as described herein.

cGAS Inhibitors

In some embodiments, the cGAS-STING pathway inhibitor is a cGAS inhibitor. Such inhibitors can specifically target cGAS by, for example, reducing cGAS activity (e.g., cGAMP catalysis), inhibiting binding of cGAS to mtDNA, or reducing cGAS expression. The generation of cGAMP by cGAS requires that the enzyme bind to dsDNA and use its two substrates, ATP and GTP, to generate the cyclic dinucleotide, cGAMP. Thus, a cGAS inhibitor could reduce cGAS activity by, for example, disrupting dsDNA binding, blocking ATP and/or GTP from entering the active site, or inhibiting the generation of phosphodiester linkages between ATP and GTP to prevent cyclization of cGAMP.

In some embodiments, the cGAS inhibitor is a compound described in WO2017176812.

In some embodiments, the cGAS inhibitor is a compound of formula (IV)

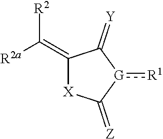

wherein the following definitions are provided in relation to the substitution of the compound of formula (IV) including formula (IVa), (IVb), and (IVc):
X is NH or S;
Y is O or S;
Z is O, S, CHR^{1a} or NR^{1a};
R^{1a} is hydrogen, C₁₋₆alkyl, or C₁₋₆alkyl selectively functionalized with one or more halogen, thiol, hydroxyl, carbonyl, carboxyl, carbonyloxyl, C₁₋₆alkoxy, C₁₋₆hydroxyalkoxy, amino, C₁₋₆alkylamino, di(C₁₋₆alkyl)amino, or azido groups;
G is N or C;
if G is N, R¹ is hydrogen C₁₋₆alkyl, or C₁₋₆alkyl selectively functionalized with one or more halogen, thiol, hydroxyl, carbonyl, carboxyl, carbonyloxyl, C₁₋₆alkoxy, C₁₋₆hydroxyalkoxy, amino, C₁₋₆alkylamino, di(C₁₋₆alkyl)amino, or azido groups,
if G is N and if Z includes R^{1a}, R¹-R^{1a} is connected as a —CH₂CH₂—, —CH₂CH₂CH₂—, —CH═CH—, —C(CH₃)═CH— or —CH═C(CH₃)— group; and
if G is C and if Z includes R^{1a}, R¹-R^{1a} is connected as a ═CH—CH═CH—, ═N—CH═CH—, or ═CH—N═CH— group;
R¹ is hydrogen or C₁₋₆alkyl, or R¹-R^{1a} are connected form a —CH₂CH₂—, —CH₂CH₂CH₂—, —CH═CH—, —C(CH₃)═CH—, or —CH═C(CH₃)— group or together with carbon or nitrogen atoms to which they are attached form a pyridine, pyrimidine or pyrazine ring;
R² is hydrogen, halogen, C₁₋₆alkyl, or C₁₋₆alkyl selectively functionalized with one or more halogen, thiol, hydroxyl, carbonyl, carboxyl, carbonyloxyl, C₁₋₆alkoxy, C₁₋₆hydroxyalkoxy, amino, C₁₋₆alkylamino, di(C₁₋₆alkyl)amino, or azido groups;
R^{2a} is phenyl or a heteroaryl group selected from the group consisting of imidazolyl, pyridyl, pyridizinyl, pyrimidinyl, and pyrazinyl, wherein the phenyl or heterocyclic group is optionally substituted with 1-4 substituents independently selected from halo the group consisting of halogen, —SR^{3a}, —S(O)R^{3a}, —OR^{3a}, —OCH₂R^{3b}, —OCH(CH₃)R^{3b}, —OC(O)NHR^{3a}, —NR^{3a}R⁴, —NHSO₂R^{3a}, azido, —CHO, —CO₂R^{3a}, cyano, C₁₋₆alkyl or —CR^{5a}R^{6a}R^{7a}, C₂₋₆alkenyl, —C(R^{5a})═C(R^{8a})(R^{9a}), C₂₋₆alkynyl, and —C≡CR^{8a};
R^{3a}, R^{3b}, and R^{4a} are independently hydrogen, phenyl, naphthyl, pyridyl, pyrimidinyl, imidazolyl, 1,2,3-triazolyl, quinolinyl, isoquinolinyl, thiazolyl, tetrazolyl groups, C₁₋₆alkyl, cyclic —(C₁₋₈alkyl)-, cyclic —(C₁₋₆oxaalkyl)-, cyclic —(C₁₋₆azaalkyl)-, C₂₋₆alkenyl, or C₂₋₆alkynyl;
wherein the phenyl, naphthyl, pyridyl, pyrimidinyl, imidazolyl, 1,2,3-triazolyl, quinolinyl, isoquinolinyl, thiazolyl, or tetrazolyl groups are optionally substituted with 1-3 substituents independently selected from the group consisting of halogen, thiol, C₁₋₆alkyl thioether, C₁₋₆alkyl sulfoxide, C₁₋₆alkyl, C₁₋₆alkoxyl, amino, C₁₋₆alkylamino, C₁₋₆dialkylamino, C₁₋₆alkyl sulfonamide, azido, —CHO, —CO₂H, C₁₋₆alkyl carboxylate, cyano, C₂₋₆alkenyl, and C₂₋₆alkynyl group; and the C₁₋₆alkyl, cyclic —(C₁₋₈alkyl)-, cyclic —(C₁₋₆oxaalkyl)-, cyclic —(C₁₋₄azaalkyl)-, C₂₋₆alkenyl, or C₂₋₆alkynyl groups are selectively functionalized with one or more halogen, thiol, hydroxyl, carbonyl, carboxyl, carbonyloxyl, C₁₋₆alkoxy, C₁₋₆hydroxyalkoxy, amino, C₁₋₆alkylamino, di(C₁₋₆alkyl)amino, azido, piperidinyl, phenyl, naphthyl, pyridyl, pyrimidinyl, imidazolyl, 1,2,3-triazolyl, quinolinyl, isoquinolinyl, thiazolyl, or tetrazolyl groups; and
R^{5a}, R^{6a}, R^{7a}, R^{8a} and R^{9a} are independently hydrogen, phenyl, naphthyl, pyridyl, pyrimidinyl, imidazolyl, 1,2,3-triazolyl, quinolinyl, isoquinolinyl, thiazolyl, tetrazolyl groups, C₁₋₆alkyl, cyclic —(C₁₋₈alkyl)-, cyclic —(C₁₋₆oxaalkyl)-, cyclic —(C₁₋₆azaalkyl)-, C₂₋₆alkenyl, C₂₋₆alkynyl, C₁₋₆alkoxyl, cyclic —(C₁₋₈alkoxyl)-, cyclic —(C₁₋₆oxaalkoxyl)-, cyclic —(C₁₋₆azaalkoxyl)-;
wherein the phenyl, naphthyl, pyridyl, pyrimidinyl, imidazolyl, 1,2,3-triazolyl, quinolinyl, isoquinolinyl, or thiazolyl, tetrazolyl groups are optionally substituted with 1-3 substituents independently selected from the group consisting of halogen, thiol, C₁₋₆alkyl thioether, C₁₋₆alkyl sulfoxide, C₁₋₆ alkyl, C₁₋₆alkoxyl, amino, C₁₋₆alkylamino, C₁₋₆dialkylamino, C₁₋₆alkyl sulfonamide, azido, —CHO, —CO₂H, C₁₋₆alkyl carboxylate, cyano, C₂₋₆alkenyl, and C₂₋₆alkynyl group, and the C₁₋₆alkyl, cyclic —(C₁₋₈alkyl)-, cyclic —(C₁₋₆oxaalkyl)-, cyclic —(C₁₋₆azaalkyl)-, C₂₋₆alkenyl, or C₂₋₆alkynyl groups are selectively functionalized with one or more halogen, thiol, hydroxyl, carbonyl, carboxyl, carbonyloxyl, C₁₋₆alkoxy, C₁₋₆hydroxyalkoxy, amino, C₁₋₆alkylamino, di(C₁₋₆alkyl)amino, azido, piperidinyl, phenyl, naphthyl, pyridyl, pyrimidinyl, imidazolyl, 1,2,3-triazolyl, quinolinyl, isoquinolinyl, thiazolyl, or tetrazolyl groups,
   or a pharmaceutically acceptable salt, solvate, stereoisomer, tautomer, or prodrug thereof

In some embodiments, X is S, Y is O or S, and R^{2a} is a imidazolyl, pyridyl, pyridizinyl, pyrimidinyl, or pyrazinyl group with 0-3 substituents independently selected from the group consisting of halogen, —SR^{3a}, —S(O)R^{3a}, —OR^{3a}, —OCH₂R^{3b}, —OCH(CH₃)R^{3b}, —OC(O)NHR^{3a}, —NR^{3a}R^{4a}, —NHSO₂R^{3a}, azido, carbonyl, —CHO, CO₂R^{3a}, cyano, C₁₋₆alkyl or —CR^{5a}R^{6a}R^{7a}, C₂₋₆alkeny, —C(R^{5a})═C(R^{8a})(R^{9a}), C₂₋₆alkynyl, and —C≡CR^{8a}

In some embodiments, X is S, Y is O or S, and R^{2a} is imidazolyl group with 0-3 substituents independently selected from the group consisting of halogen, —SR^{3a}, —S(O)R^{3a}, —OR^{3a}, —OCH₂R^{3b}, —OCH(CH₃)R^{3b}, —OC(O)NHR^{3a}, —NR^{3a}R^{4a}, —NHSO₂R^{3a}, azido, —CHO, CO₂R^{3a}, cyano, C₁₋₆alkyl or —CR^{5a}R^{6a}R^{7a}, C₂₋₆alkeny, —C(R^{5a})═C(R^{8a})(R^{9a}), C₂₋₆alkynyl, and —C≡CR^{8a}.

In some embodiments, X is S, Y is O or S, and R^{2a} is pyridyl group with 0-3 substituents independently selected from the group consisting of halogen, —SR^{3a}, —S(O)R^{3a}, —OR^{3a}, —OCH₂R^{3b}, —OCH(CH₃)R^{3b}, —OC(O)NHR^{3a}, —NR^{3a}R^{4a}, —NHSO₂R^{3a}, azido, —CHO, CO₂R^{3a}, cyano, C₁₋₆alkyl or —CR^{5a}R^{6a}R^{7a}, C₂₋₆alkeny, —C(R^{5a})═C(R^{8a})(R^{9a}), C₂₋₆alkynyl, and —C≡CR^{8a}.

In some embodiments, X is S, Y is O or S, and R^{2a} is pyridizinyl group with 0-3 substituents independently selected from the group consisting of halogen, —SR^{3a}, —S(O)R^{3a}, —OR^{3a}, —OCH₂R^{3b}, —OCH(CH₃)R^{3b}, —OC(O)NHR^{3a}, —NR^{3a}R^{4a}, —NHSO₂R^{3a}, azido, —CHO, CO₂R^{3a}, cyano, C₁₋₆alkyl or —CR^{5a}R^{6a}R^{7a}, C₂₋₆alkeny, —C(R^{5a})═C(R^{8a})(R^{9a}), C₂₋₆alkynyl, and —C≡CR^{8a}.

In some embodiments, X is S, Y is O or S, and R^{2a} is pyrimidinyl group with 0-3 substituents independently selected from the group consisting of halogen, —SR^{3a}, —S(O)R^{3a}, —OR^{3a}, —OCH₂R^{3b}, —OCH(CH₃)R^{3b}, —OC(O)NHR^{3a}, —NR^{3a}R⁴, —NHSO₂R^{3a}, azido, —CHO, CO₂R^{3a}, cyano, C₁₋₆alkyl or —CR^{5a}R^{6a}R^{7a}, C₂₋₆alkeny, —C(R^{5a})═C(R^{8a})(R^{9a}), C₂₋₆alkynyl, and —C≡CR^{8a}.

In some embodiments, X is S, Y is O or S, and R^{2a} is pyrazinyl group with 0-3 substituents independently selected from the group consisting of halogen, —SR^{3a}, —S(O)R^{3a}, —OR^{3a}, —OCH₂R^{3b}, —OCH(CH₃)R^{3b}, —OC(O)NHR^{3a}, —NR^{3a}R^{4a}, —NHSO₂R^{3a}, azido, —CHO, CO₂R^{3a}, cyano, C₁₋₆alkyl or —CR^{5a}R^{6a}R^{7a}, C₂₋₆alkeny, —C(R^{5a})═C(R^{8a})(R^{9a}), C₂₋₆alkynyl, and —C≡CR^{8a}.

In some embodiments, X is S, Y is O or S, and R^{2a} is phenyl group with 0-4 substituents independently selected from the group consisting of halogen, —SR^{3a}, —S(O)R^{3a}, —OR^{3a}, —OCH₂R^{3b}, —OCH(CH₃)R^{3b}, —OC(O)NHR^{3a}, —NR^{3a}R^{4a}, —NHSO₂R^{3a}, azido, —CHO, CO₂R^{3a}, cyano, C₁₋₆alkyl or —CR^{5a}R^{6a}R^{7a}, C₂₋₆alkeny, —C(R^{5a})═C(R^{5a})(R^{5a}), C₂₋₆alkynyl, and —C≡CR^{8a}.

In some embodiments, X is S, Y is O or S, G is N, and R¹ is hydrogen C₁₋₆alkyl, or C₁₋₆alkyl selectively functionalized with one or more halogen, thiol, hydroxyl, carbonyl, carboxyl, carbonyloxyl, C₁₋₆alkoxy, C₁₋₆hydroxyalkoxy, amino, C₁₋₆alkylamino, di(C₁₋₆alkyl)amino, or azido groups.

In some embodiments, X is S, Y is O or S, G is N, and R¹-R^{1a} is connected as a —CH₂CH₂—, —CH₂CH₂CH₂—, —CH═CH—, —C(CH₃)═CH—, or —CH═C(CH₃)— group.

In some embodiments, X is S, Y is O or S, G is C, Z is NR^{1a}, and R¹-R^{1a} is connected as a ═CH—CH═CH—, ═N—CH═CH—, or ═CH—N═CH— group.

In some embodiments, X is S, Y is O or S, and R² is hydrogen, halogen, C₁₋₆alkyl, or C₁₋₆alkyl selectively functionalized with one or more halogen, thiol, hydroxyl, carbonyl, carboxyl, carbonyloxyl, C₁₋₆alkoxy, C₁₋₆hydroxyalkoxy, amino, C₁₋₆alkylamino, di(C₁₋₆alkyl)amino, or azido groups.

In some embodiments, R² is hydrogen, Cl, Br, or methyl.

In some embodiments, the cGAS inhibitor compound of Formula (IVa)

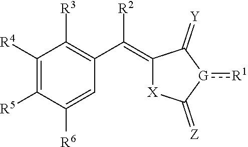

wherein:

### X is NH or S;

### Y is O or S;

Z is O, S, CHR^{1a} or NR^{1a};
R^{1a} is hydrogen, C₁₋₆alkyl, or C₁₋₆alkyl selectively functionalized with one or more halogen, thiol, hydroxyl, carbonyl, carboxyl, carbonyloxyl, C₁₋₆alkoxy, C₁₋₆hydroxyalkoxy, amino, C₁₋₆alkylamino, di(C₁₋₆alkyl)amino, or azido groups; G is N or C;
if G is N, R¹ is hydrogen C₁₋₆alkyl, or C₁₋₆alkyl selectively functionalized with one or more halogen, thiol, hydroxyl, carbonyl, carboxyl, carbonyloxyl, C₁₋₆alkoxy, C₁₋₆hydroxyalkoxy, amino, C₁₋₆alkylamino, di(C₁₋₆alkyl)amino, or azido groups, or R¹-R^{1a} is connected as a —CH₂CH₂—, —CH₂CH₂CH₂—, —CH═CH—, —C(CH₃)═CH— or —CH═C(CH₃)— group; and if G is C, R¹-R^{1a} is connected as a ═CH—CH═CH—, ═N—CH═CH—, or ═CH—N═CH— group;
R¹ is hydrogen or C₁₋₆alkyl, or R¹-R^{1a} are connected form a —CH₂CH₂—, —CH₂CH₂CH₂—, —CH═CH—, —C(CH₃)═CH—, or —CH═C(CH₃)— group or together with carbon or nitrogen atoms to which they are attached form a pyridine, pyrimidine or pyrazine ring;
R² is hydrogen, halo, C₁₋₆alkyl, or C₁₋₆alkyl selectively functionalized with one or more halogen, thiol, hydroxyl, carbonyl, carboxyl, carbonyloxyl, C₁₋₆alkoxy, C₁₋₆hydroxyalkoxy, amino, C₁₋₆alkylamino, di(C₁₋₆alkyl)amino, or azido groups;
R³, R⁵, and R⁶ are independently hydrogen, halogen, —SR^{3a}, —S(O)R^{3a}, —OR^{3a}, —OCH₂R^{3b}, —OCH(CH₃)R^{3b}, —OC(O)NHR^{3a}, —NR^{3a}R^{4a}, —NHSO₂R^{3a}, azido, —CHO, CO₂R^{3a}, cyano, C₁₋₆alkyl or —CR^{5a}R^{6a}R^{7a}, C₂₋₆alkeny, —C(R^{5a})═C(R^{8a})(R^{9a}), C₂₋₆ alkynyl, —C≡CR^{8a}, or R²-R³ are connected as a —CH₂CH₂— or —CH₂CH₂CH₂— group;
R^{3a}, R^{3b}, and R⁴⁸ are independently hydrogen, phenyl, naphthyl, pyridyl, pyrimidinyl, imidazolyl, 1,2,3-triazolyl, quinolinyl, isoquinolinyl, thiazolyl, tetrazolyl groups, C₁₋₆alkyl, cyclic-(C₁₋₈alkyl)-, cyclic —(C₁₋₆oxaalkyl)-, cyclic —(C₁₋₆azaalkyl)-, C₂₋₆alkenyl, C₂₋₆alkynyl;
wherein the phenyl, naphthyl, pyridyl, pyrimidinyl, imidazolyl, 1,2,3-triazolyl, quinolinyl, isoquinolinyl, thiazolyl, or tetrazolyl groups are optionally substituted with 1-3 substituents independently selected from the group consisting of halogen, thiol, C₁₋₆alkyl thioether, C₁₋₆alkyl sulfoxide, C₁₋₆alkyl, C₁₋₆alkoxyl, amino, C₁₋₆alkylamino, C₁₋₆dialkylamino, C₁₋₆alkyl sulfonamide, azido, —CHO, —CO₂H, C₁₋₆alkyl carboxylate, cyano, C₂₋₆alkeny, and C₂₋₆alkynyl group; and the C₁₋₆alkyl, cyclic —(C₁₋₈alkyl)-, cyclic —(C₁₋₆oxaalkyl)-, cyclic —(C₁₋₆azaalkyl)-, C₂₋₆alkenyl, or C₂₋₆ alkynyl groups are selectively functionalized with one or more halogen, thiol, hydroxyl, carbonyl, carboxyl, carbonyloxyl, C₁₋₆alkoxy, C₁₋₆hydroxyalkoxy, amino, C₁₋₆alkylamino, di(C₁₋₆alkyl)amino, azido, piperidinyl, phenyl, naphthyl, pyridyl, pyrimidinyl, imidazolyl, 1,2,3-triazolyl, quinolinyl, isoquinolinyl, thiazolyl, or tetrazolyl groups;
R⁴ is hydrogen or halogen,
or a pharmaceutically acceptable salt, solvate, stereoisomer, tautomer, or prodrug thereof.

In some embodiments, X is S, Y is O or S, G is N, and R¹ is hydrogen C₁₋₆alkyl, or C₁₋₆alkyl selectively functionalized with one or more halogen, thiol, hydroxyl, carbonyl, carboxyl, carbonyloxyl, C₁₋₆alkoxy, C₁₋₆hydroxyalkoxy, amino, C₁₋₆alkylamino, di(C₁₋₆alkyl)amino, or azido groups.

In some embodiments, X is S, Y is O or S, G is N, and R¹-R^{1a} is connected as a —CH₂CH₂—, —CH₂CH₂CH₂—, —CH═CH—, —C(CH₃)═CH—, or —CH═C(CH₃)— group.

In some embodiments, X is S, Y is O or S, G is C, Z is NR^{1a}, and R¹-R^{1a} is connected as a ═CH—CH═CH—, ═N—CH═CH—, or ═CH—N═CH— group.

In some embodiments, R^{2a} is hydrogen, Cl, Br, or methyl.

In some embodiments, X is S, Y is O or S, G is N, and R¹ is hydrogen C₁₋₆alkyl, or C₁₋₆alkyl selectively functionalized with one or more halogen, thiol, hydroxyl, carbonyl, carboxyl, carbonyloxyl, C₁₋₆alkoxy, C₁₋₆hydroxyalkoxy, amino, C₁₋₆alkylamino, di(C₁₋₆alkyl)amino, or azido groups.

In some embodiments, X is S, Y is O or S, G is C, Z is NR^{1a}, and R¹-R^{1a} is connected as a ═CH—CH═CH—, ═N—CH═CH—, or ═CH—N═CH— group.

In some embodiments, the cGAS inhibitor is a compound of formula (IVb)

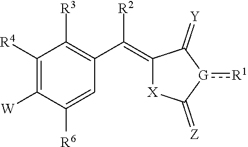

wherein:

### X is NH or S;

### Y is O or S;

Z is O, S, CHR^{1a} or NR^{1a};
R¹¹ is hydrogen, C₁₋₆alkyl, or C₁₋₆alkyl selectively functionalized with one or more halogen, thiol, hydroxyl, carbonyl, carboxyl, carbonyloxyl, C₁₋₆alkoxy, C₁₋₆hydroxyalkoxy, amino, C₁₋₆alkylamino, di(C₁₋₆alkyl)amino, or azido groups;

### G is N or C;

if G is N, R¹ is hydrogen C₁₋₆alkyl, or C₁₋₆alkyl selectively functionalized with one or more halogen, thiol, hydroxyl, carbonyl, carboxyl, carbonyloxyl, C₁₋₆alkoxy, C₁₋₆hydroxyalkoxy, amino, C₁₋₆alkylamino, di(C₁₋₆alkyl)amino, or azido groups, or R¹-R^{1a} is connected as a —CH₂CH₂—, —CH₂CH₂CH₂—, —CH═CH—, —C(CH₃)═CH— or —CH═C(CH₃)— group; and if G is C, R¹-R^{1a} is connected as a ═CH—CH═CH—, ═N—CH═CH—, or ═CH—N═CH— group;
W is OR^{10a} or NHR^{10a};
wherein R^{10a} is hydrogen, C₁₋₆alkyl, C₁₋₆alkyl selectively functionalized with one or more halogen, thiol, hydroxyl, carbonyl, carboxyl, carbonyloxyl, C₁₋₆ alkoxy, C₁₋₆hydroxyalkoxy, amino, C₁₋₆alkylamino, di(C₁₋₆alkyl)amino, or azido groups, or R^{10a}-R⁶ is connected as a —CH₂—CH₂—, —CH═CH—, —N═CH—, or —CH═N— group;
R² is hydrogen, halo, C₁₋₆alkyl, or C₁₋₆alkyl selectively functionalized with one or more halogen, thiol, hydroxyl, carbonyl, carboxyl, carbonyloxyl, C₁₋₆alkoxy, C₁₋₆hydroxyalkoxy, amino, C₁₋₆alkylamino, di(C₁₋₆alkyl)amino, or azido groups;
R³ and R⁶ are independently hydrogen, halogen, —SR^{3a}, —S(O)R^{3a}, —OR^{3a}, —OCH₂R^{3b}, —OCH(CH₃)R^{3b}, —OC(O)NHR^{3a}, —NR^{3a}R^{4a}, —NHSO₂R^{3a}, azido, —CHO, CO₂R^{3a}, cyano, C₁₋₆alkyl or —CR^{5a}R^{6a}R^{7a}, C₂₋₆alkeny, —C(R^{5a})═C(R^{8a})(R^{9a}), C₂₋₆alkynyl, —C≡CR^{8a}, or R²-R³ are connected as a —CH₂CH₂— or —CH₂CH₂CH₂— group;
R^{3a}, R^{3b}, and R^{4a} are independently hydrogen, phenyl, naphthyl, pyridyl, pyrimidinyl, imidazolyl, 1,2,3-triazolyl, quinolinyl, isoquinolinyl, thiazolyl, tetrazolyl groups, C₁₋₆alkyl, cyclic-(C₁₋₈alkyl)-, cyclic —(C₁₋₆oxaalkyl)-, cyclic —(C₁₋₆azaalkyl)-, C₂₋₆alkenyl, C₂₋₆alkynyl;
wherein the phenyl, naphthyl, pyridyl, pyrimidinyl, imidazolyl, 1,2,3-triazolyl, quinolinyl, isoquinolinyl, thiazolyl, or tetrazolyl groups are optionally substituted with 1-3 substituents independently selected from the group consisting of halogen, thiol, C₁₋₆alkyl thioether, C₁₋₆alkyl sulfoxide, C₁₋₆alkyl, C₁₋₆alkoxyl, amino, C₁₋₆alkylamino, C₁₋₆dialkylamino, C₁₋₆alkyl sulfonamide, azido, —CHO, —CO₂H, C₁₋₆alkyl carboxylate, cyano, C₂₋₆alkeny, and C₂₋₆alkynyl group; and the C₁₋₆alkyl, cyclic —(C₁₋₈alkyl)-, cyclic —(C₁₋₆oxaalkyl)-, cyclic —(C₁₋₆azaalkyl)-, C₂₋₆alkenyl, or C₂₋₆ alkynyl groups are selectively functionalized with one or more halogen, thiol, hydroxyl, carbonyl, carboxyl, carbonyloxyl, C₁₋₆alkoxy, C₁₋₆hydroxyalkoxy, amino, C₁₋₆alkylamino, di(C₁₋₆alkyl)amino, azido, piperidinyl, phenyl, naphthyl, pyridyl, pyrimidinyl, imidazolyl, 1,2,3-triazolyl, quinolinyl, isoquinolinyl, thiazolyl, or tetrazolyl groups;
R⁴ is hydrogen or halogen,

or a pharmaceutically acceptable salt, solvate, stereoisomer, tautomer, or prodrug thereof.

In some embodiments, X is S, Y is O or S, Z is O or S, G is N, and R¹ is hydrogen C₁₋₆alkyl, or C₁₋₆alkyl selectively functionalized with one or more halogen, thiol, hydroxyl, carbonyl, carboxyl, carbonyloxyl, C₁₋₆alkoxy, C₁₋₆hydroxyalkoxy, amino, C₁₋₆alkylamino, di(C₁₋₆alkyl)amino, or azido groups.

In some embodiments, X is S, Y is O or S, G is N, Z is NR^{1a}, and R¹-R^{1a} is connected as a —CH₂CH₂—, —CH₂CH₂CH₂—, —CH═CH—, —C(CH₃)═CH—, or —CH═C(CH₃)— group.

In some embodiments, X is S, Y is O or S, G is C, Z is NR^{1a}, and R¹-R^{1a} is connected as a ═CH—CH═CH—, ═N—CH═CH—, or ═CH—N═CH— group.

In some embodiments, R² is hydrogen, Cl, Br, or methyl.

In some embodiments, the cGAS inhibitor is a compound of formula (IVc)

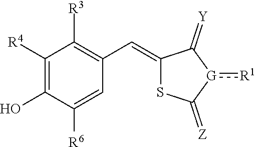

wherein:
Z is O, S, CHR^{1a} or NR^{1a};
R^{1a} is hydrogen, C₁₋₆alkyl, or C₁₋₆alkyl selectively functionalized with one or more halogen, thiol, hydroxyl, carbonyl, carboxyl, carbonyloxyl, C₁₋₆alkoxy, C₁₋₆hydroxyalkoxy, amino, C₁₋₆alkylamino, di(C₁₋₄alkyl)amino, or azido groups;

### G is N or C;

if G is N, R¹ is hydrogen C₁₋₆alkyl, or C₁₋₆alkyl selectively functionalized with one or more halogen, thiol, hydroxyl, carbonyl, carboxyl, carbonyloxyl, C₁₋₆ alkoxy, C₁₋₆hydroxyalkoxy, amino, C₁₋₆alkylamino, di(C₁₋₆alkyl)amino, or azido groups, or R¹-R^{1a} is connected as a —CH₂CH₂—, —CH₂CH₂CH₂—, —CH═CH—, —C(CH₃)═CH— or —CH═C(CH₃)— group; and if G is C, R¹-R^{1a} is connected as a ═CH—CH═CH—, ═N—CH═CH—, or ═CH—N═CH— group;
R³ and R⁴ are independently hydrogen or halogen;
R⁶ is hydrogen, halogen, —SR^{3a}, —S(O)R^{3a}, —OR^{3a}, —OCH₂R^{3b}, —OCH(CH₃)R^{3b}, —OC(O)NHR^{3a}, —NR^{3a}R^{4a}, —NHSO₂R^{3a}, azido, —CHO, CO₂R^{3a}, cyano, C₁₋₆alkyl or —CR^{5a}R^{6a}R^{7a}, C₂₋₆alkeny, —C(R^{5a})═C(R^{8a})(R^{9a}), C₂₋₆alkynyl, —C≡CR^{8a}, or R²-R³ are connected as a —CH₂CH₂— or —CH₂CH₂CH₂— group;
R^{3a}, R^{3b}, and R^{4a} are independently hydrogen, phenyl, naphthyl, pyridyl, pyrimidinyl, imidazolyl, 1,2,3-triazolyl, quinolinyl, isoquinolinyl, thiazolyl, tetrazolyl groups, C₁₋₆alkyl, cyclic-(C₁₋₈alkyl)-, cyclic —(C₁₋₆oxaalkyl)-, cyclic —(C₁₋₆azaalkyl)-, C₂₋₆alkenyl, C₂₋₆alkynyl;
wherein the phenyl, naphthyl, pyridyl, pyrimidinyl, imidazolyl, 1,2,3-triazolyl, quinolinyl, isoquinolinyl, thiazolyl, or tetrazolyl groups are optionally substituted with 1-3 substituents independently selected from the group consisting of halogen, thiol, C₁₋₆alkyl thioether, C₁₋₆alkyl sulfoxide, C₁₋₆alkyl, C₁₋₆alkoxyl, amino, C₁₋₆alkylamino, C₁₋₆dialkylamino, C₁₋₆alkyl sulfonamide, azido, —CHO, —CO₂H, C₁₋₆alkyl carboxylate, cyano, C₂₋₆alkeny, and C₂₋₆alkynyl group; and the C₁₋₆alkyl, cyclic —(C₁₋₈alkyl)-, cyclic —(C₁₋₆oxaalkyl)-, cyclic —(C₁₋₆azaalkyl)-, C₂₋₆alkenyl, or C₂₋₆ alkynyl groups are selectively functionalized with one or more halogen, thiol, hydroxyl, carbonyl, carboxyl, carbonyloxyl, C₁₋₆alkoxy, C₁₋₆hydroxyalkoxy, amino, C₁₋₆alkylamino, di(C₁₋₆alkyl)amino, azido, piperidinyl, phenyl, naphthyl, pyridyl, pyrimidinyl, imidazolyl, 1,2,3-triazolyl, quinolinyl, isoquinolinyl, thiazolyl, or tetrazolyl groups,
or a pharmaceutically acceptable salt, solvate, stereoisomer, tautomer, or prodrug thereof.

In some embodiments, Z is O or S, G is N, and R¹ is hydrogen C₁₋₆alkyl, or C₁₋₆alkyl selectively functionalized with one or more halogen, thiol, hydroxyl, carbonyl, carboxyl, carbonyloxyl, C₁₋₆alkoxy, C₁₋₆hydroxyalkoxy, amino, C₁₋₆alkylamino, di(C₁₋₆alkyl)amino, or azido groups.

In some embodiments, G is N, Z is NR^{1a}, and R¹-R^{1a} is connected as a —CH₂CH₂—, —CH₂CH₂CH₂—, —CH═CH—, —C(CH₃)═CH—, or —CH═C(CH₃)— group.

In some embodiments, G is C, Z is NR^{1a}, and R¹-R^{1a} is connected as a ═CH—CH═CH—, ═N—CH═CH—, or ═CH—N═CH— group.

In some embodiments, G is N, R¹ is methyl, Z is O, R³ and R⁴ are independently hydrogen or halogen, and R⁶ is —OR^{3a}, —OCH₂R^{3b}, or —OCH(CH₃)R^{3b};

wherein R^{3a} and R^{3b} are independently hydrogen, phenyl, naphthyl, pyridyl, pyrimidinyl, imidazolyl, 1,2,3-triazolyl, quinolinyl, isoquinolinyl, thiazolyl, tetrazolyl groups, C₁₋₆alkyl, cyclic-(C₁₋₈alkyl)-, cyclic —(C₁₋₈oxaalkyl)-, cyclic —(C₁₋₄azaalkyl)-, C₂₋₆alkenyl, C₂₋₆alkynyl;
wherein the phenyl, naphthyl, pyridyl, pyrimidinyl, imidazolyl, 1,2,3-triazolyl, quinolinyl, isoquinolinyl, thiazolyl, or tetrazolyl groups are optionally substituted with 1-3 substituents independently selected from the group consisting of halogen, thiol, C₁₋₆alkyl thioether, C₁₋₆alkyl sulfoxide, C₁₋₆alkyl, C₁₋₆alkoxyl, amino, C₁₋₆alkylamino, C₁₋₆alkylamino, C₁₋₆alkyl sulfonamide, azido, —CHO, —CO₂H, C₁₋₆alkyl carboxylate, cyano, C₂₋₆alkeny, and C₂₋₆alkynyl group; and the C₁₋₆alkyl, cyclic —(C₁₋₈alkyl)-, cyclic —(C₁₋₆oxaalkyl)-, cyclic —(C₁₋₆azaalkyl)-, C₂₋₆alkenyl, or C₂₋₆ alkynyl groups are selectively functionalized with one or more halogen, thiol, hydroxyl, carbonyl, carboxyl, carbonyloxyl, C₁₋₆alkoxy, C₁₋₆hydroxyalkoxy, amino, C₁₋₆alkylamino, di(C₁₋₆alkyl)amino, azido, piperidinyl, phenyl, naphthyl, pyridyl, pyrimidinyl, imidazolyl, 1,2,3-triazolyl, quinolinyl, isoquinolinyl, thiazolyl, or tetrazolyl groups.

In some embodiments, the cGAS inhibitor is a compound selected from the group consisting of

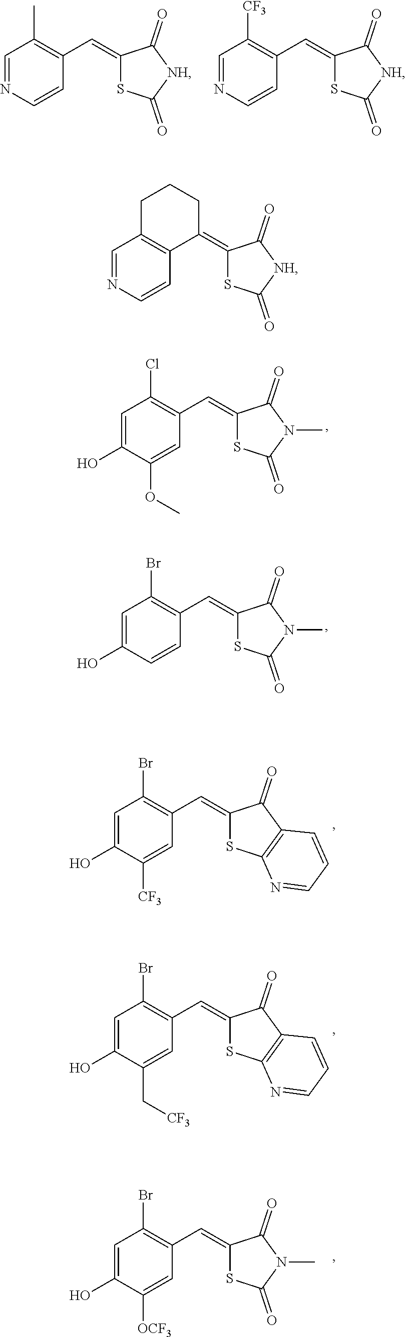

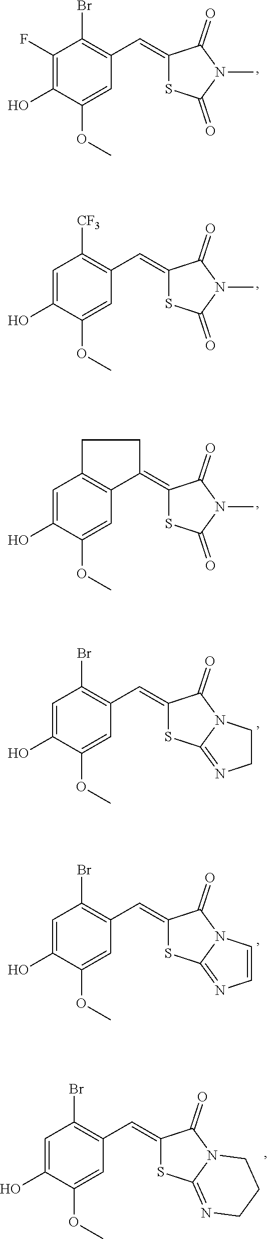 [SMILES: COc1cc(/C=C2\SC(=O)N(C)C2=O)c(Br)c(F)c1O ; COc1cc(/C=C2\SC(=O)N(C)C2=O)c(C(F)(F)F)cc1O ; COc1cc2c(cc1O)CC/C2=C1/SC(=O)N(C)C1=O ; COc1cc(/C=c2\sc3n(c2=O)CCN=3)c(Br)cc1O ; COc1cc(/C=c2\sc3nccn3c2=O)c(Br)cc1O ; COc1cc(/C=c2\sc3n(c2=O)CCCN=3)c(Br)cc1O]

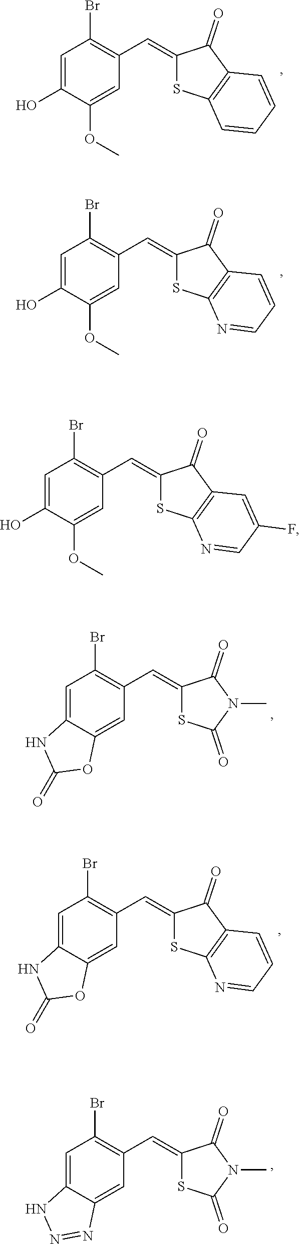 [SMILES: COc1cc(/C=C2\Sc3ccccc3C2=O)c(Br)cc1O ; COc1cc(/C=C2\Sc3ncccc3C2=O)c(Br)cc1O ; COc1cc(/C=C2\Sc3ncc(F)cc3C2=O)c(Br)cc1O ; CN1C(=O)S/C(=C\c2cc3oc(=O)[nH]c3cc2Br)C1=O ; O=C1/C(=C/c2cc3oc(=O)[nH]c3cc2Br)Sc2ncccc21 ; CN1C(=O)S/C(=C\c2cc3nn[nH]c3cc2Br)C1=O]

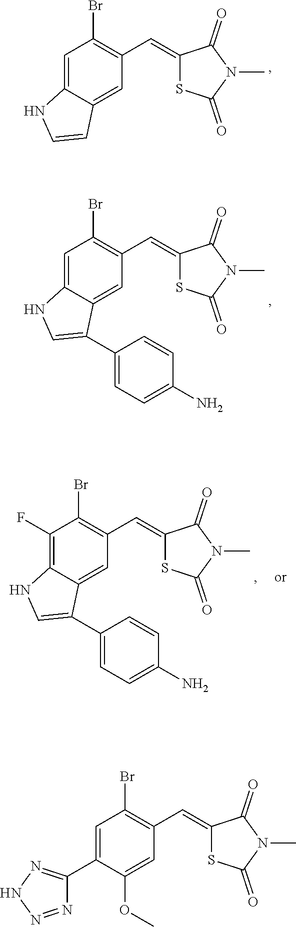 [SMILES: CN1C(=O)S/C(=C\c2cc3cc[nH]c3cc2Br)C1=O ; CN1C(=O)S/C(=C\c2cc3c(-c4ccc(N)cc4)c[nH]c3cc2Br)C1=O ; CN1C(=O)S/C(=C\c2cc3c(-c4ccc(N)cc4)c[nH]c3c(F)c2Br)C1=O ; COc1cc(/C=C2\SC(=O)N(C)C2=O)c(Br)cc1-c1nn[nH]n1]

In some embodiments, the cGAS inhibitor is a compound selected from the group consisting of

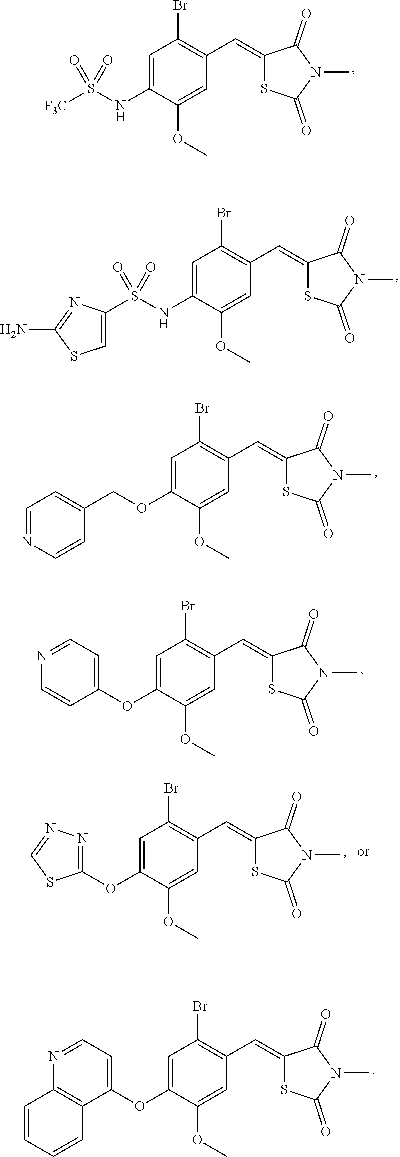 [SMILES: COc1cc(/C=C2\SC(=O)N(C)C2=O)c(Br)cc1NS(=O)(=O)C(F)(F)F ; COc1cc(/C=C2\SC(=O)N(C)C2=O)c(Br)cc1NS(=O)(=O)c1csc(N)n1 ; COc1cc(/C=C2\SC(=O)N(C)C2=O)c(Br)cc1OCc1ccncc1 ; COc1cc(/C=C2\SC(=O)N(C)C2=O)c(Br)cc1Oc1ccncc1 ; COc1cc(/C=C2\SC(=O)N(C)C2=O)c(Br)cc1Oc1nncs1 ; COc1cc(/C=C2\SC(=O)N(C)C2=O)c(Br)cc1Oc1ccnc2ccccc12]

In some embodiments, the cGAS inhibitor is a compound selected from the group consisting of

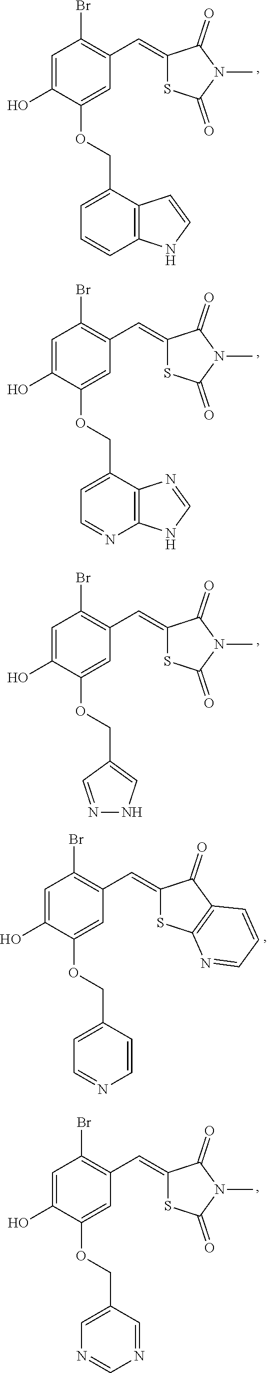

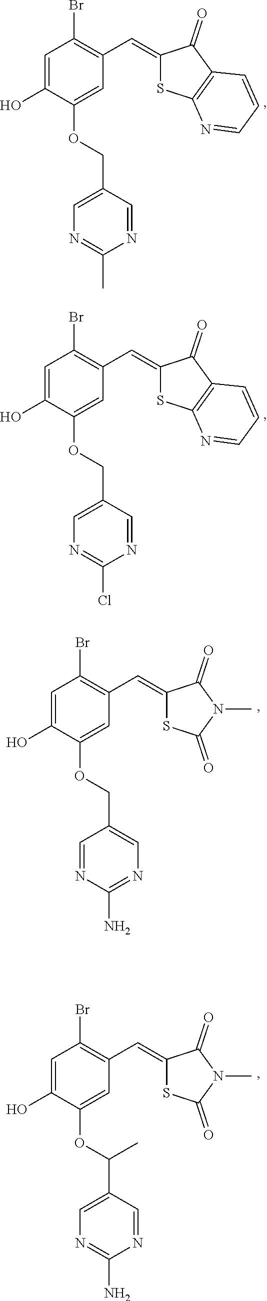

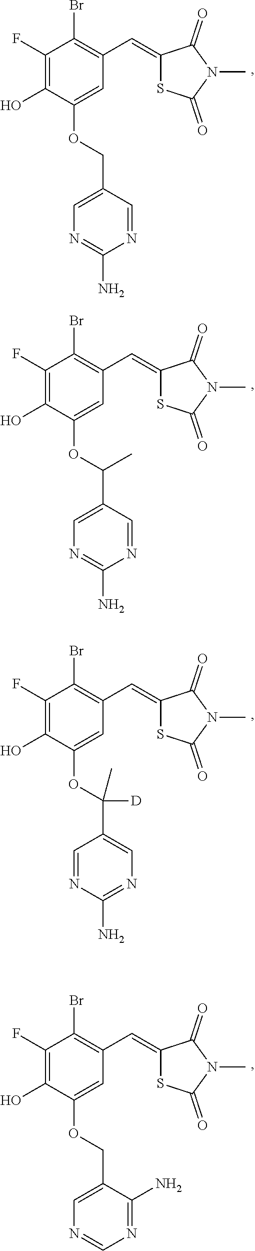

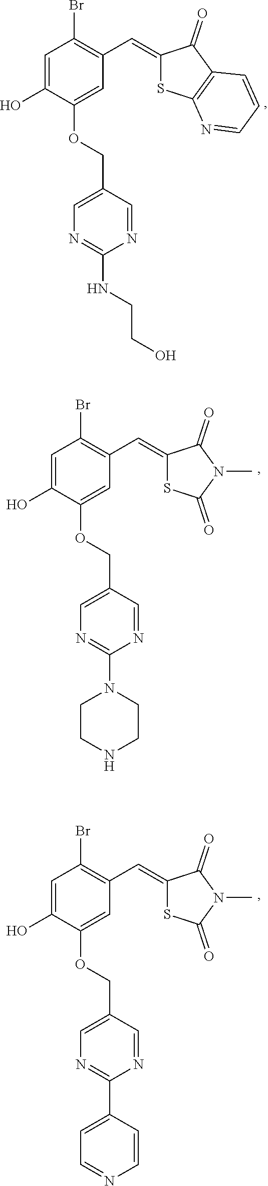 [SMILES: O=C1/C(=C/c2cc(OCc3cnc(NCCO)nc3)c(O)cc2Br)Sc2ncccc21 ; CN1C(=O)S/C(=C\c2cc(OCc3cnc(N4CCNCC4)nc3)c(O)cc2Br)C1=O ; CN1C(=O)S/C(=C\c2cc(OCc3cnc(-c4ccncc4)nc3)c(O)cc2Br)C1=O]

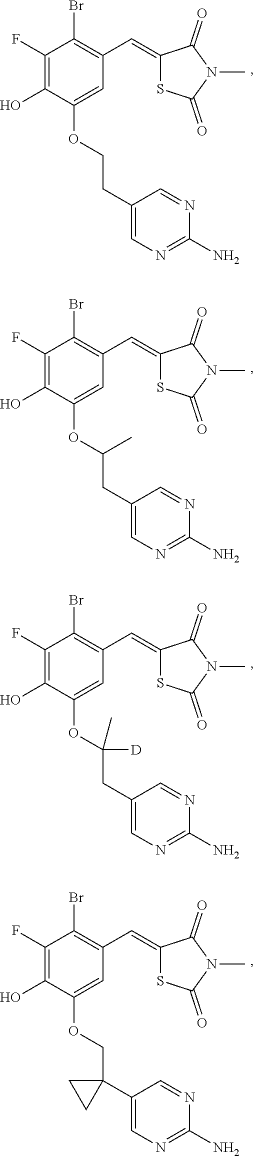 [SMILES: CN1C(=O)S/C(=C\c2cc(OCCc3cnc(N)nc3)c(O)c(F)c2Br)C1=O ; CC(Cc1cnc(N)nc1)Oc1cc(/C=C2\SC(=O)N(C)C2=O)c(Br)c(F)c1O ; [2H]C(C)(Cc1cnc(N)nc1)Oc1cc(/C=C2\SC(=O)N(C)C2=O)c(Br)c(F)c1O ; CN1C(=O)S/C(=C\c2cc(OCC3(c4cnc(N)nc4)CC3)c(O)c(F)c2Br)C1=O]

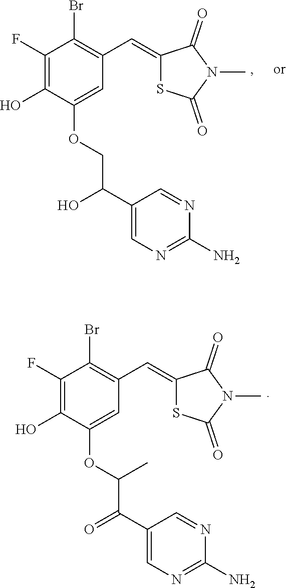 [SMILES: CN1C(=O)S/C(=C\c2cc(OCC(O)c3cnc(N)nc3)c(O)c(F)c2Br)C1=O ; CC(Oc1cc(/C=C2\SC(=O)N(C)C2=O)c(Br)c(F)c1O)C(=O)c1cnc(N)nc1]

Structural studies have shown that upon dsDNA binding, cGAS is activated through conformational transitions resulting in the formation of a catalytically competent and accessible nucleotide-binding pocket for generation of cGAMP (Gao et al., 2013). Thus, in some embodiments, the cGAS inhibitor binds to the catalytic site of cGAS. Arg364 and Tyr421 are two highly conserved catalytic site amino-acid residues found in mouse and human cGAS. Point mutations in these residues render cGAS incapable of responding to dsDNA and abolish downstream inflammatory signalling (Gao et al., 2013). Accordingly, in some embodiments, the cGAS inhibitor interacts with Arg364 and/or Tyr421 of cGAS. In some embodiments, the cGAS inhibitor is a compound described in Gao et al. (2013). In one embodiment, the cGAS inhibitor is RU.521, as described in Gao et al. (2013).

In one embodiment, the cGAS inhibitor may be a compound of formula (V) or a pharmaceutically acceptable salt, solvate, stereoisomer, tautomer, or prodrug thereof:

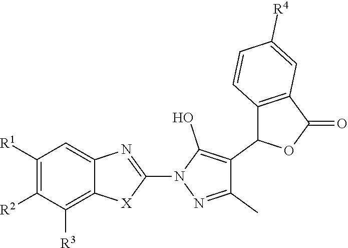

wherein R¹ to R⁴ and X are defined as follows in relation to formula (V).

X is O, S, or NR⁵.

R¹ to R⁴ are each independently selected from the group consisting of hydrogen, halo, C₁₋₁₀alkyl, C₁₋₁₀alkyenyl, C₂₋₁₀alkynyl, CN, NO₂, OC(O)R⁵, C(O)R⁵, C(O)NR⁵R⁶, C(O)OR⁵, OR⁶, OS(O)₂R⁵, NR⁵R⁶, SR⁵, wherein R⁵ and R⁶ are each independently selected from hydrogen, C₁₋₁₀alkyl, C₂₋₁₀alkenyl, C₂₋₁₀alkynyl and C₁₋₁₀alkylhalo;

In one embodiment, R¹ may hydrogen or halogen. R¹ may be hydrogen, chlorine, bromine or fluorine.

In one embodiment, R² and R³ may be hydrogen, halogen or C₁₋₁₀alkyl. R² and R³ may be hydrogen, bromine, chlorine, fluorine or methyl. For example, R² and R³ may be chlorine.

In one embodiment, R⁴ may be hydrogen, halogen or methoxy.

In one embodiment, X may be NH or S.

In one embodiment, R¹ is hydrogen, R² and R³ are each independently chlorine, R⁴ is hydrogen, and X is NH. In one preferred embodiment, the cGAS inhibitor may be a compound of the following formula:

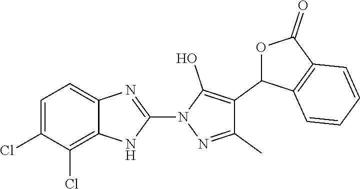 [SMILES: Cc1nn(-c2nc3ccc(Cl)c(Cl)c3[nH]2)c(O)c1C1OC(=O)c2ccccc21]

or a pharmaceutically acceptable salt, solvate, stereoisomer, tautomer, or prodrug thereof.

In some embodiments, the cGAS inhibitor is a compound described in Hall et al. (2017). In some embodiments, the cGAS inhibitor is PF-06928215 or a pharmaceutically acceptable salt, solvate, stereoisomer, tautomer, or prodrug thereof. In some embodiments, the cGAS inhibitor is a compound of the following formula:

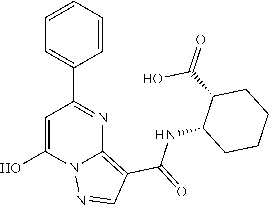 [SMILES: O=C(N[C@H]1CCCC[C@H]1C(=O)O)c1cnn2c(O)cc(-c3ccccc3)nc12]

In some embodiments the cGAS inhibitor is a polynucleotide. For instance, the polynucleotide may inhibit cGAS by reducing the expression of cGAS. In some embodiments, the polynucleotide reduces expression of cGAS by RNA interference. In some embodiments, the polynucleotide is an siRNA. Exemplary siRNAs that can be used to target cGAS mRNA are commercially available, e.g., from ThermoFisher (siRNA IDs: s41748, s41746, and s41747).

In some embodiments, the cGAS inhibitor is a polypeptide. In some embodiments, the polypeptide comprises an antigen binding site of an antibody which binds to cGAS. In some embodiments, the cGAS inhibitor is an antibody or a fragment thereof. Antibodies that target STING are commercially available from various sources such as Santa Cruz Biotechnology (e.g., Cat. No. sc-515777). Such antibodies can be used to derive other polypeptides comprising an antigen binding site using routine methods, such as, for example scFv antibodies, intrabodies or nanobodies as described herein.

cGAMP Inhibitors

In some embodiments, the cGAS-STING pathway inhibitor is a cGAMP inhibitor. Such inhibitors can specifically target cGAMP by, for example, sequestering/binding to cGAMP to prevent it from interacting with STING and thereby inhibiting downstream transcription of inflammatory cytokines.

In some embodiments, the cGAMP inhibitor binds to cGAMP. In some embodiments, the cGAMP inhibitor is a polypeptide comprising an antigen binding site of an antibody which binds to cGAMP. Suitable polypeptides comprising antigen binding sites are described herein. In some embodiments the cGAMP inhibitor is an antibody, or fragment thereof, that binds to cGAMP. Suitable antibodies are described in Hall et al. (2017) and WO2018045369. Thus, in an embodiment, the cGAMP inhibitor is a polypeptide comprising the antigen binding site of any one of the antibodies described in Hall et al. (2017) or WO2018045369.

Administration of cGAS-STING Pathway Inhibitors

In some embodiments, a method for treating or preventing a TDP-43 proteinopathy in a subject, includes administration of a pharmaceutical composition containing at least one cGAS-STING pathway inhibitor, or a pharmaceutically acceptable salt, pharmaceutically acceptable N-oxide, pharmaceutically active metabolite, pharmaceutically acceptable prodrug, or pharmaceutically acceptable solvate thereof, in therapeutically effective amounts to said subject.

A cGAS-STING pathway inhibitor, is administered to treat, prevent, or at least partially arrest the symptoms of a subject already suffering from and/or diagnosed as having a TDP-43 proteinopathy, e.g., ALS or FTD. Amounts effective for this use will depend on the severity and course of the disease, previous therapy, the subject's health status, weight, and response to the treatment. It is considered well within the skill of the art for one to determine such therapeutically effective amounts by routine experimentation (including, but not limited to, a dose escalation clinical trial).

In preventative applications, compositions containing a cGAS-STING pathway inhibitor are administered to a subject susceptible to or otherwise at risk of developing an TDP-43 proteinopathy, e.g., ALS or FTD. Such an amount is defined to be a “prophylactically effective amount or dose” i.e., a dose sufficient to prevent or reduce the onset of neurodegenerative symptoms. In this use, the precise amounts also depend on the particular disease, the subject's state of health, weight, timing, etc. It is considered well within the skill of the art for one to determine such prophylactically effective amounts by routine experimentation (e.g., a dose escalation clinical trial).

In a case where a subject's status does improve, upon reliable medical advice, the administration of a cGAS-STING pathway inhibitor may be given continuously; alternatively, the dose of drug being administered may be temporarily reduced or temporarily suspended for a certain length of time (i.e., a “drug holiday”). The length of the drug holiday can vary between 2 days and 1 year, including by way of example only, 2 days, 3 days, 4 days, 5 days, 6 days, 7 days, 10 days, 12 days, 15 days, 20 days, 28 days, 35 days, 50 days, or 60 days. The dose reduction during a drug holiday may be from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

The amount of a given cGAS-STING pathway inhibitor that will be suitable as a therapeutically effective dose will vary depending upon factors such as the type and potency of the cGAS-STING pathway inhibitor to be administered, the severity/stage of the subject's disease, the characteristics (e.g., weight) of the subject in need of treatment, and prior or concurrent treatments, but can nevertheless be routinely determined in a manner known in the art according to the particular circumstances surrounding the case, including, e.g., the specific agent being administered, the route of administration, the condition being treated or prevented, and the subject or host being treated. In general, however, doses employed for adult human treatment will typically be in the range of 0.02-5000 mg per day, or from about 1-1500 mg per day. The desired dose may conveniently be presented in a single dose or as divided doses administered simultaneously (or over a short period of time) or at appropriate intervals, for example as two, three, four or more sub-doses per day.

The foregoing ranges are merely suggestive, as the number of variables in regard to an individual treatment regime is large, and considerable excursions from these recommended values are not uncommon. Such dosages may be altered depending on a number of variables, not limited to the activity of the cGAS-STING pathway inhibitor to be used, the type and severity of TDP-43 proteinopathy to be treated or prevented, the mode of administration, and the judgment of the practitioner.

Toxicity and therapeutic efficacy of such therapeutic regimens can be determined by standard pharmaceutical procedures in cell cultures or experimental animals, including, but not limited to, the determination of the LD₅₀ (the dose lethal to 50% of the population) and the ED₅₀ (the dose therapeutically effective in 50% of the population). The dose ratio between the toxic and therapeutic effects is the therapeutic index and it can be expressed as the ratio between LD₅₀ and ED₅₀. cGAS-STING pathway inhibitors exhibiting high therapeutic indices are preferred. The data obtained from cell culture assays and animal studies can be used in formulating a range of dosage for use in human and non-human subjects. The dosage of such compounds lies preferably within a range of circulating concentrations that include the ED₅₀ with minimal toxicity. The dosage may vary within this range depending upon the dosage form employed and the route of administration utilized.

In some embodiments, the cGAS-STING pathway inhibitor is administered in an amount sufficient to reduce or prevent an increase in neuroinflammation. As used herein, the term “neuroinflammation” refers to inflammation of nervous tissue, such as brain or spinal cord tissue. Neuroinflammation is caused in response to a wide variety of inflammatory cytokines, including NF-κB related cytokines and type I interferons. In TDP-43 proteinopathies, it is believed that neuroinflammation contributes to neurodegeneration which in turn leads to progressive worsening of symptoms. Thus, in some embodiments, the cGAS-STING pathway inhibitor is administered in an amount which is sufficient to reduce or prevent an increase in neurodegeneration. Such amounts may reduce or prevent an increase in the severity of symptoms of TDP-43 proteinopathies, such as muscle weakness, atrophy, muscle spasms, reduced motor skills, speech impairment, memory loss, and cognitive or behavioral dysfunction.

In some embodiments, the cGAS-STING pathway inhibitor is administered in an amount sufficient to reduce or prevent an increase in type I interferon expression. Activation of the cGAS-STING pathway results in production of type I interferons, which in turn is considered to cause neuroinflammation, thereby contributing to the neurodegenerative symptoms of TDP-43 proteinopathies, such as ALS. Thus, in some embodiments, a therapeutically effective amount of a cGAS-STING pathway inhibitor is an amount which reduces or prevents an increase in type I interferon expression.

Type I interferons (IFNs) are a large subgroup of interferon proteins that help regulate the activity of the immune system. The mammalian types are designated IFN-α (alpha), IFN-β (beta), IFN-κ (kappa), IFN-δ (delta), IFN-ε (epsilon), IFN-τ (tau), IFN-ω (omega), and IFN-ζ (zeta, also known as limitin). Accordingly, type I interferon expression can be quantified by measuring the expression of any one or more of these type I IFN subtypes. In some embodiments, the expression of IFN-β is measured. Thus, in some embodiments, the cGAS-STING pathway inhibitor is administered in an amount sufficient to reduce or prevent an increase in IFN-β expression. Expression levels can be assessed using routine methods by measuring either mRNA levels (e.g., by RT-qPCR) or protein levels (e.g., by an ELISA). Suitable methods are described herein and in Seeds and Miller (2011).

In some embodiments, the cGAS-STING pathway inhibitor is administered in an amount sufficient to reduce or prevent an increase in NF-κB related cytokine expression. As used herein, the phrase “NF-κB related cytokine”, refers to any cytokine whose production is increased in response to NF-κB mediated activity. Such NF-κB related cytokines include TNFα, IL-1α, IL-1β, and IL-6. Activation of the cGAS-STING pathway results in production of NF-κB related cytokines, which in turn are considered to cause neuroinflammation, thereby contributing to the neurodegenerative symptoms of TDP-43 proteinopathies, such as ALS. Thus, in some embodiments, the cGAS-STING pathway inhibitor is administered in an amount sufficient to reduce or prevent an increase in expression of any one or more of TNFα, IL-1α, IL-1β, and IL-6. In some embodiments, the expression of TNFα is reduced or prevented from increasing. Expression levels of NF-κB related cytokines can be assessed using routine methods by measuring either mRNA levels (e.g., by RT-qPCR) or protein levels (e.g., by an ELISA). For example, expression levels of TNFα can be measured using commercially available assays for quantifying TNFα such as the “Human TNF alpha Assay Kit” available from Cisbio (cat no. 62HTNFAPEG) or the “TNF alpha Human ELISA Kit” available from ThermoFisher (cat no. KHC3011).

### Combination Treatments

cGAS-STING pathway inhibitors can also be used in combination with other agents of therapeutic value in the treatment or prevention of TDP-43 proteinopathies, e.g., ALS and FTD. In some embodiments, the other agent is one which is conventionally used to treat or prevent neurodegenerative diseases. In general, other agents do not necessarily have to be administered in the same pharmaceutical composition, and may, because of different physical and chemical characteristics, preferably be administered by different routes. The determination of the mode of administration and the advisability of administration, where possible, in the same pharmaceutical composition, is well within the knowledge of the skilled clinician. The initial administration can be made according to established protocols known in the art, and then, based upon the observed effects, the dosage, modes of administration and times of administration can be modified by the skilled clinician.

A cGAS-STING pathway inhibitor and an additional therapeutic agent may be administered concurrently (e.g., simultaneously, essentially simultaneously or within the same treatment protocol) or sequentially, depending upon the nature and phase of the infection, the condition of the subject, and the actual choice of therapeutic agents used. The determination of the order of administration, and the number of repetitions of administration of each therapeutic agent during a treatment protocol, is well within the knowledge of the skilled physician after evaluation of the disease being treated or prevented and the condition of the subject.

It is known to those of skill in the art that therapeutically-effective dosages can vary when the drugs are used in treatment combinations. Methods for experimentally determining therapeutically-effective dosages of drugs and other agents for use in combination treatment regimens are described in the literature. For example, the use of metronomic dosing, i.e., providing more frequent, lower doses in order to minimize toxic side effects, has been described extensively in the literature. Combination treatment further includes periodic treatments that start and stop at various times to assist with the clinical management of the subject.

For combination therapies, dosages of co-administered therapeutic agents will of course vary depending on the type of co-agents employed, on the specific cGAS-STING pathway inhibitor, and TDP-43 proteinopathy to be treated or prevented.

It is understood that the dosage regimen to treat, prevent, or ameliorate the condition(s) for which relief is sought, can be modified in accordance with a variety of factors. These factors include the condition from which the subject suffers, as well as the age, weight, sex, diet, and general medical condition of the subject. Thus, the dosage regimen actually employed can vary widely and therefore can deviate from the dosage regimens set forth herein.

The cGAS-STING pathway inhibitor and additional therapeutic agent which make up a combination therapy disclosed herein may be a combined dosage form or in separate dosage forms intended for substantially simultaneous administration. The pharmaceutical agents that make up the combination therapy may also be administered sequentially, with either therapeutic compound being administered by a regimen calling for two-step administration. The two-step administration regimen may call for sequential administration of the active agents or spaced-apart administration of the separate active agents. The time period between the multiple administration steps may range from, a few minutes to several hours, depending upon the properties of each pharmaceutical agent, such as potency, solubility, bioavailability, plasma half-life and kinetic profile of the pharmaceutical agent. Circadian variation of various physiological parameters may also be evaluated to determine the optimal dose interval.

In addition, administration or co-administration of a cGAS-STING pathway inhibitor for treatment or prevention of a TDP-43 proteinopathy may be used in combination with procedures that may provide additional or synergistic benefit to the subject. By way of example only, subjects may undergo genetic testing to identify genetic variation in their genome so as to optimize treatment parameters, e.g., the type of cGAS-STING pathway inhibitor to be administered, dosing regimen, and co-administration with additional therapeutic agents.

Initial administration can be via any route practical, such as, for example, an intravenous injection, a bolus injection, infusion over 5 minutes to about 5 hours, a pill, a capsule, inhaler, injection, transdermal patch, buccal delivery, and the like, or combination thereof. A compound should be administered as soon as is practicable after the onset of a disease or condition is detected or suspected, and for a length of time necessary for the treatment or prevention of the TDP-43 proteinopathy.

In some embodiments, the cGAS-STING pathway inhibitor is administered in combination with a therapy that is conventionally used to treat or prevent neurodegenerative disease. For instance, the other therapy can be an antidepressant, a neuroleptic, an anticonvulsant, medroxyprogestrone, a dopaminergic agent, a cholinesterase inhibitor, riluzole, and/or edaravone.

In some embodiments, the cGAS-STING pathway inhibitor is administered in combination with an anti-inflammatory agent. In some embodiments, the anti-inflammatory agent is a non-steroidal anti-inflammatory drug (NSAID). In some embodiments, the anti-inflammatory agent is a cytokine inhibitor, for example a molecule that binds to TNFα, IL-6, IL-1, or IFNβ and prevents them from binding to their receptor.

### Dosage Forms

Compositions comprising cGAS-STING pathway inhibitors can be formulated for administration to a subject via any conventional means including, but not limited to, oral, parenteral (e.g., intravenous, subcutaneous, or intramuscular), buccal, inhalation, intranasal, rectal or transdermal administration routes.

The pharmaceutical compositions which include a cGAS-STING pathway inhibitor alone or in combination with one or more other therapeutic agents, can be formulated into any suitable dosage form, including but not limited to, aqueous oral dispersions, liquids, mists, gels, syrups, elixirs, slurries, suspensions and the like, for oral ingestion by a subject to be treated, solid oral dosage forms, aerosols, controlled release formulations, fast melt formulations, effervescent formulations, lyophilized formulations, tablets, powders, pills, dragees, capsules, delayed release formulations, extended release formulations, pulsatile release formulations, multiparticulate formulations, and mixed immediate release and controlled release formulations.

Pharmaceutical preparations for oral use can be obtained by mixing one or more solid excipient with one or more of the compounds, optionally grinding the resulting mixture, and processing the mixture of granules, after adding suitable auxiliaries, if desired, to obtain tablets or dragee cores. Suitable excipients include, for example, fillers such as sugars, including lactose, sucrose, mannitol, or sorbitol; cellulose preparations such as, for example, maize starch, wheat starch, rice starch, potato starch, gelatin, gum tragacanth, methylcellulose, microcrystalline cellulose, hydroxypropylmethylcellulose, sodium carboxymethylcellulose; or others such as: polyvinylpyrrolidone (PVP or povidone) or calcium phosphate. If desired, disintegrating agents may be added, such as the cross-linked croscarmellose sodium, polyvinylpyrrolidone, agar, or alginic acid or a salt thereof such as sodium alginate.

In another aspect, dosage forms may include microencapsulated formulations. In some embodiments, one or more other compatible materials are present in the microencapsulation material. Exemplary materials include, but are not limited to, pH modifiers, erosion facilitators, anti-foaming agents, antioxidants, flavoring agents, and carrier materials such as binders, suspending agents, disintegration agents, filling agents, surfactants, solubilizers, stabilizers, lubricants, wetting agents, and diluents.

Microencapsulated formulations of a cGAS-STING pathway inhibitor may be formulated by methods known by one of ordinary skill in the art. Such known methods include, e.g., spray drying processes, spinning disk-solvent processes, hot melt processes, spray chilling methods, fluidized bed, electrostatic deposition, centrifugal extrusion, rotational suspension separation, polymerization at liquid-gas or solid-gas interface, pressure extrusion, or spraying solvent extraction bath. In addition to these, several chemical techniques, e.g., complex coacervation, solvent evaporation, polymer-polymer incompatibility, interfacial polymerization in liquid media, in situ polymerization, in-liquid drying, and desolvation in liquid media could also be used. Furthermore, other methods such as roller compaction, extrusion/spheronization, coacervation, or nanoparticle coating may also be used.

The pharmaceutical solid oral dosage forms including formulations can be further formulated to provide a controlled release of the cGAS-STING pathway inhibitor. Controlled release refers to the release of one or more active agents from a dosage form in which they are incorporated according to a desired profile over an extended period of time. Controlled release profiles include, for example, sustained release, prolonged release, pulsatile release, and delayed release profiles. In contrast to immediate release compositions, controlled release compositions allow delivery of an agent to a subject over an extended period of time according to a predetermined profile. Such release rates can provide therapeutically effective levels of agent for an extended period of time and thereby provide a longer period of pharmacologic response while minimizing side effects as compared to conventional rapid release dosage forms. Such longer periods of response provide for many inherent benefits that are not achieved with the corresponding short acting, immediate release preparations.

In some embodiments, the solid dosage forms can be formulated as enteric coated delayed release oral dosage forms, i.e., as an oral dosage form of a pharmaceutical composition which utilizes an enteric coating to affect release in the small intestine of the gastrointestinal tract. The enteric coated dosage form may be a compressed or molded or extruded tablet/mold (coated or uncoated) containing granules, powder, pellets, beads or particles of the active ingredient and/or other composition components, which are themselves coated or uncoated. The enteric coated oral dosage form may also be a capsule (coated or uncoated) containing pellets, beads or granules of the solid carrier or the composition, which are themselves coated or uncoated.

The term “delayed release” as used herein refers to the delivery so that the release can be accomplished at some generally predictable location in the intestinal tract more distal to that which would have been accomplished if there had been no delayed release alterations. In some embodiments the method for delay of release is coating. Any coatings should be applied to a sufficient thickness such that the entire coating does not dissolve in the gastrointestinal fluids at pH below about 5, but does dissolve at pH about 5 and above. It is expected that any anionic polymer exhibiting a pH-dependent solubility profile can be used as an enteric coating in the methods and compositions to achieve delivery to the lower gastrointestinal tract. In some embodiments the polymers are anionic carboxylic polymers.

In some embodiments, the coating can, and usually does, contain a plasticizer and possibly other coating excipients such as colorants, talc, and/or magnesium stearate, which are well known in the art. Suitable plasticizers include triethyl citrate (Citroflex 2), triacetin (glyceryl triacetate), acetyl triethyl citrate (Citroflec A2), Carbowax 400 (polyethylene glycol 400), diethyl phthalate, tributyl citrate, acetylated monoglycerides, glycerol, fatty acid esters, propylene glycol, and dibutyl phthalate. In particular, anionic carboxylic acrylic polymers usually will contain 10-25% by weight of a plasticizer, especially dibutyl phthalate, polyethylene glycol, triethyl citrate and triacetin. Conventional coating techniques such as spray or pan coating are employed to apply coatings. The coating thickness must be sufficient to ensure that the oral dosage form remains intact until the desired site of topical delivery in the intestinal tract is reached.

Colorants, detackifiers, surfactants, antifoaming agents, lubricants (e.g., camuba wax or PEG) may be added to the coatings besides plasticizers to solubilize or disperse the coating material, and to improve coating performance and the coated product.

In other embodiments, the cGAS-STING pathway inhibitor formulations are delivered using a pulsatile dosage form. A pulsatile dosage form is capable of providing one or more immediate release pulses at predetermined time points after a controlled lag time or at specific sites. Pulsatile dosage forms may be administered using a variety of pulsatile formulations known in the art. For example, such formulations include, but are not limited to, those described in U.S. Pat. Nos. 5,011,692, 5,017,381, 5,229,135, and 5,840,329. Other pulsatile release dosage forms suitable for use with the present formulations include, but are not limited to, for example, U.S. Pat. Nos. 4,871,549, 5,260,068, 5,260,069, 5,508,040, 5,567,441 and 5,837,284. In one embodiment, the controlled release dosage form is pulsatile release solid oral dosage form including at least two groups of particles, (i.e. multiparticulate) each containing a formulation. The first group of particles provides a substantially immediate dose of the cGAS-STING pathway inhibitor upon ingestion. The first group of particles can be either uncoated or include a coating and/or sealant. The second group of particles includes coated particles, which includes from about 2% to about 75%, from about 2.5% to about 70%, or from about 40% to about 70%, by weight of the total dose of the active agents in the formulation, in admixture with one or more binders. The coating includes a pharmaceutically acceptable ingredient in an amount sufficient to provide a delay of from about 2 hours to about 7 hours following ingestion before release of the second dose. Suitable coatings include one or more differentially degradable coatings such as, by way of example only, pH sensitive coatings (enteric coatings) such as acrylic resins either alone or blended with cellulose derivatives, e.g., ethylcellulose, or non-enteric coatings having variable thickness to provide differential release of the formulation.

Many other types of controlled release systems known to those of ordinary skill in the art and are suitable for use with the formulations described herein. Examples of such delivery systems include, e.g., polymer-based systems, such as polylactic and polyglycolic acid, plyanhydrides and polycaprolactone; porous matrices, nonpolymer-based systems that are lipids, including sterols, such as cholesterol, cholesterol esters and fatty acids, or neutral fats, such as mono-, di- and triglycerides; hydrogel release systems; silastic systems; peptide-based systems; wax coatings, bioerodible dosage forms, compressed tablets using conventional binders and the like. See, e.g., U.S. Pat. Nos. 4,327,725, 4,624,848, 4,968,509, 5,461,140, 5,456,923, 5,516,527, 5,622,721, 5,686,105, 5,700,410, 5,977,175, 6,465,014 and 6,932,983.

Liquid formulation dosage forms for oral administration can be aqueous suspensions selected from the group including, but not limited to, pharmaceutically acceptable aqueous oral dispersions, emulsions, solutions, elixirs, gels, and syrups.

The aqueous suspensions and dispersions can remain in a homogenous state, as defined in The USP Pharmacists' Pharmacopeia (2005 edition, chapter 905), for at least 4 hours. The homogeneity should be determined by a sampling method consistent with regard to determining homogeneity of the entire composition. In one embodiment, an aqueous suspension can be re-suspended into a homogenous suspension by physical agitation lasting less than 1 minute. In another embodiment, an aqueous suspension can be re-suspended into a homogenous suspension by physical agitation lasting less than 45 seconds. In yet another embodiment, an aqueous suspension can be re-suspended into a homogenous suspension by physical agitation lasting less than 30 seconds. In still another embodiment, no agitation is necessary to maintain a homogeneous aqueous dispersion.

In addition to the additives listed above, the liquid formulations can also include inert diluents commonly used in the art, such as water or other solvents, solubilizing agents, and emulsifiers. Exemplary emulsifiers are ethyl alcohol, isopropyl alcohol, ethyl carbonate, ethyl acetate, benzyl alcohol, benzyl benzoate, propyleneglycol, 1,3-butyleneglycol, dimethylformamide, sodium lauryl sulfate, sodium doccusate, cholesterol, cholesterol esters, taurocholic acid, phosphotidylcholine, oils, such as cottonseed oil, groundnut oil, corn germ oil, olive oil, castor oil, and sesame oil, glycerol, tetrahydrofurfuryl alcohol, polyethylene glycols, fatty acid esters of sorbitan, or mixtures of these substances, and the like.

### Injectable Formulations

Formulations suitable for intramuscular, subcutaneous, or intravenous injection may include physiologically acceptable sterile aqueous or non-aqueous solutions, dispersions, suspensions or emulsions, and sterile powders for reconstitution into sterile injectable solutions or dispersions. Examples of suitable aqueous and non-aqueous carriers, diluents, solvents, or vehicles including water, ethanol, polyols (propyleneglycol, polyethylene-glycol, glycerol, cremophor and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions, and by the use of surfactants. Formulations suitable for subcutaneous injection may also contain additives such as preserving, wetting, emulsifying, and dispensing agents. Prevention of the growth of microorganisms can be ensured by various antibacterial and antifungal agents, such as parabens, chlorobutanol, phenol, sorbic acid, and the like. It may also be desirable to include isotonic agents, such as sugars, sodium chloride, and the like. Prolonged absorption of the injectable pharmaceutical form can be brought about by the use of agents delaying absorption, such as aluminum monostearate and gelatin.

For intravenous injections, compounds may be formulated in aqueous solutions, preferably in physiologically compatible buffers such as Hank's solution, Ringer's solution, or physiological saline buffer. For transmucosal administration, penetrants appropriate to the barrier to be permeated are used in the formulation. Such penetrants are generally known in the art. For other parenteral injections, appropriate formulations may include aqueous or nonaqueous solutions, preferably with physiologically compatible buffers or excipients. Such excipients are generally known in the art.

Parenteral injections may involve bolus injection or continuous infusion. Formulations for injection may be presented in unit dosage form, e.g., in ampoules or in multi-dose containers, with an added preservative. The pharmaceutical composition may be in a form suitable for parenteral injection as a sterile suspensions, solutions or emulsions in oily or aqueous vehicles, and may contain formulatory agents such as suspending, stabilizing and/or dispersing agents. Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions. Alternatively, the active ingredient may be in powder form for constitution with a suitable vehicle, e.g., sterile pyrogen-free water, before use.

The pharmaceutical compositions may be in unit dosage forms suitable for single administration of precise dosages. In unit dosage form, the formulation is divided into unit doses containing appropriate quantities of one or more compound. The unit dosage may be in the form of a package containing discrete quantities of the formulation. Non-limiting examples are packaged tablets or capsules, and powders in vials or ampoules. Aqueous suspension compositions can be packaged in single-dose non-reclosable containers. Alternatively, multiple-dose reclosable containers can be used, in which case it is typical to include a preservative in the composition. By way of example only, formulations for parenteral injection may be presented in unit dosage form, which include, but are not limited to ampoules, or in multi-dose containers, with an added preservative.

### Diagnostic Methods

The present inventors have identified the cGAS-STING pathway as the innate immune sensing pathway which promotes neuroinflammation in TDP-43 proteinopathies such as ALS and FTD. This surprising finding opens up previously unavailable avenues for diagnosing such diseases.

Accordingly, in an aspect, the present invention provides a method of diagnosing a subject with a TDP-43 proteinopathy, the method comprising detecting activation of the cGAS-STING pathway in a sample from the subject. Similarly, the present invention also provides a method for determining a likelihood of responsiveness to treatment with a cGAS-STING pathway inhibitor in a subject suffering from a TDP-43 proteinopathy, the method comprising detecting activation of the cGAS-STING pathway in a sample from the subject, wherein activation of the cGAS-STING pathway indicates a higher likelihood of responsiveness to the treatment.

Detecting activation of the cGAS-STING pathway may involve measuring the activity of any of the components of the cGAS-STING pathway or their downstream signalling targets. For example, in some embodiments, detecting activation of the cGAS-STING pathway comprises measuring the level of cGAMP in a sample from the subject. cGAMP is a secondary messenger that is produced by cGAS upon detection of cytosolic DNA and stimulates STING to activate TBK1 which in turn drives expression of inflammatory cytokines. Accordingly, elevated cGAMP levels are indicative of a TDP-43 proteinopathy or a higher likelihood of responsiveness to treatment with a cGAS-STING pathway inhibitor. cGAMP is a soluble factor released from cells (Ablasser et al., 2013). Thus, cGAMP provides a suitable biomarker for measurement in a sample from a subject to identify subjects with TDP-43 proteinopathies. In some embodiments, the sample is a blood sample. In some embodiments, the sample is a urine sample. In some embodiments, the sample is a tissue sample, for example from a biopsy. In some embodiments, the tissue sample is a nerve tissue sample.

Levels of cGAMP can be measured using routine methods that are known in the art such as mass spectrometry and ELISA. Kits for measuring cGAMP levels are commercially available from, for example, Cayman chemical (cat no. 501700). Other assays for measurement of cGAMP are described in Hall et al. (2017) and WO2018045369.

A “threshold” level of cGAMP can be determined based on, e.g., comparison of a levels of cGAMP in a control sample. Suitable controls would be readily apparent to a person skilled in the art and include a sample from an individual who does not suffer from a TDP-43 proteinopathy. Suitable threshold levels can readily be determined by the skilled person using routine experimentation.

In some embodiments, detecting cGAS-STING pathway activation comprises detecting the presence of cytosolic mtDNA. In other embodiments, detecting cGAS-STING pathway activation comprises detecting type I IFN or NF-κB expression, as descried herein.

In some embodiments, the subject may be suffering from a neurodegenerative disease in which TDP-43 pathology is unknown. Accordingly, the methods described herein can be used to determine a likelihood of responsiveness to treatment with a cGAS-STING pathway inhibitor in a subject suffering from a neurodegenerative disease, the method comprising detecting activation of the cGAS-STING pathway in a sample from the subject, wherein activation of the cGAS-STING pathway indicates a higher likelihood of responsiveness to the treatment. Such methods are particularly useful for neurodegenerative diseases that can multiple underlying causes including TDP-43 mislocalisation and/or aggregation. In some embodiments, the subject is suffering from a neurodegenerative disease which is suspected to be a TDP-43 proteinopathy.

### Kits

Also provided herein are kits containing inhibitors useful for the treatment or prevention of TDP-43 proteinopathies as described above.

In one example, the kit comprises (a) a container comprising a cGAS-STING pathway inhibitor as described herein, optionally in a pharmaceutically acceptable carrier or diluent; and (b) a package insert with instructions for treating or preventing a TDP-43 proteinopathy in a subject.

In accordance with this example of the disclosure, the package insert is on or associated with the container. Suitable containers include, for example, bottles, vials, syringes, etc. The containers may be formed from a variety of materials such as glass or plastic. The container holds or contains a composition that is effective for treating or preventing the TDP-43 proteinopathy and may have a sterile access port (for example, the container may be an intravenous solution bag or a vial having a stopper pierceable by a hypodermic injection needle). At least one active agent in the composition is the cGAS-STING pathway inhibitor. The label or package insert indicates that the composition is used for administration to a subject eligible for treatment, e.g., one having or predisposed to a TDP-43 proteinopathy, with specific guidance regarding dosing amounts and intervals of compound and any other medicament being provided. The kit may further comprise an additional container comprising a pharmaceutically acceptable diluent buffer, such as bacteriostatic water for injection (BWFI), phosphate-buffered saline, Ringer's solution, and/or dextrose solution. The kit may further include other materials desirable from a commercial and user standpoint, including other buffers, diluents, filters, needles, and syringes.

### EXAMPLES

### Example 1—Materials and Methods

### Cell Culture and Transfection

Immortalised mouse embryonic fibroblasts (MEFs) lacking MAVS, PKR, cGAS, STING, Bax/Bak, Mcl1 or WT control were maintained in DME/KELSO medium (in-house DMEM containing 40 mM sodium bicarbonate, 1 mM HEPES, 0.0135 mM folic acid, 0.24 mM L-asparagine, 0.55 mM L-arginine, 1×Pen/Strep and 22.2 mM D-glucose) supplemented with 10% FBS (Sigma-Aldrich). Transfection of MEFs was performed using FuGENE HD (Promega). AGK−/− (gift from D. Stojanovski) and WT HEK293T cells were maintained in DMEM supplemented with 10% FBS, 100 U/mL Penicillin and 100 μg/mL Streptomycin. Lipofectamine 2000 (Life Technologies) was used to transfect HEK293T cells according to manufacturers' instructions. Human monocytic THP-1 cells were grown in RPMI-1640 with 10% FBS, 100 U/mL Penicillin and 100 μg/mL Streptomycin. The above cell culture was conducted at 37° C. in a humidified atmosphere with 10% CO2. Mitochondrial DNA-depleted cells (ρ0) were prepared in a relevant culture medium containing 100 ng/ml ethidium bromide, 100 μg/ml sodium pyruvate and 50 μg/ml uridine as previously demonstrated in Hashiguchi and Zhang-Akiyama (2009). The depletion was analysed using real-time qPCR to measure expression of mitochondrial DNA genes and nuclear genes (see Quantitative real-time PCR below).

### CRISPR Cas9-Mediated Gene Deletion

CRISPR/Cas9 techniques were used to generate STINGCRISPR−/− THP-1 and PpifCRISPR−/− MEFs, as has been described in Baker and Masters (2018), using targeting guide sequences as follows: hsSTING_sgRNA_FW: 5′-ucccAGA GCA CAC UCU CCG GUA CC-3′ (SEQ ID NO: 1); hsSTING_sgRNA_RV: 5′-aaacGGU ACC GGA GAG UGU GCU CU-3′ (SEQ ID NO:2); mmPpif_sgRNA_FW: 5′-ucccCGU GCC AAA GAC UGC AGG UA-3′ (SEQ ID NO:3); mmPpif_sgRNA_RV: 5′-aaacUAC CUG CAG UCU UUG GCA CG-3′ (SEQ ID NO:4). The deletion was then confirmed by western blot.

### Constructs, Reagents and Inhibitors

Third generation lentiviral constructs including pSLIK-Neo, hTDP-43 WT and Q331K (gift from Aaron D. Gitler) (Armakola et al., 2012) were used to transduce THP-1 and Mcl1−/− MEFs to generate stable cell lines carrying doxycycline-induced Flag-tagged TDP-43 following antibiotic selection with G418 (ThermoFisher Scientific). For cGAS-Flag immunoprecipitation, HEK293T cells were transfected with pMIH-Flag-mmcGAS 3 and pGW1-hTDP-43-EGFP (gift from S. Finkeiner) (Armakola et al., 2012), as well as pGW1-hTDP-43 A315T or Q331K obtained via site-directed mutagenesis using the QuickChange Lightning Kit (Agilent Technologies) and the oligos as follows: p.A315T 5′-GGA TTA ATG CTG AAC GTA CCA AAG TTC ATC CCA CCA-3′ (SEQ ID NO:5); 5′-TGG TGG GAT GAA CTT TGG TAC GTT CAG CAT TAA TCC-3′ (SEQ ID NO:6); p.Q331K 5′-CCC CAA CTG CTC TTTA GTG CTG CCT GGG C-3′ (SEQ ID NO:7); 5′-GCC CAG GCA GCA CTA AAG AGC AGT TGG GG-3′ (SEQ ID NO:8). Stimuli for cGAS/STING included: Poly(dA:dT) (Invivogen), htDNA (Sigma-Aldrich) and 2′3′-c-di-AM(PS)2(Rp, Rp) (Invivogen). Cells were co-stimulated with ABT-737 (Active Biochemicals A-1002) to trigger apoptosis as described (White et al., 2014). MitoSOX™ Red (ThermoFisher Scientific, M36008) was used to indicate superoxide production upon mitochondrial stress and analysed by flow cytometry (BD LSRFortessa X-20). To block TDP-43-mediated DNA relocation into the cytoplasm, cells were treated with cyclosporin A (Sigma-Aldrich), RU.521 (gift from M. Ascano) (Vincent et al., 2017) or H-151 (aka Life Chemicals) (Haag et al., 2018).

### Super Resolution Microscopy

MEF cell lines expressing doxycycline-induced Flagged TDP-43 were seeded onto glass coverslips (18 mm×18 mm, thickness 1½, Zeiss) for indicated times and prepared for imaging as previously described (McArthur et al., 2018). In brief, cells were fixed, blocked and incubated overnight at 4° C. with primary antibodies as follows: anti-Flag (In-house, 9H1), anti-DNA (ProGen, AC-30-10) and anti-TOM20 (Santa Cruz Biotech, sc-11415) in blocking buffer. Following two washes in PBS and an one-hour incubation with secondary antibodies [goat anti-rabbit AF647 (A21245), goat anti-mouse AF488 (A11001 and goat anti-rat AF568, A11077) from Life Technologies], coverslips were mounted onto the microscopy slide using Prolong Diamond Antifade Mountant (ThermoFisher Scientific). Three color imaging was performed on the OMX-SR system (GE Healthcare) using a 60 Å˜1.42 NA oil immersion lens (Olympus).

### Mice

TDP-43A315T transgenic mice have been described previously (Wegorzewska et al., 2009) (e.g., B6.Cg-Tg(Prnp-TARDBP*A315T)95Balo/J, JAX stock no.:010700). TDP-43T/+ mice were backcrossed for at least ten generations and then maintained on a C57BL/6 background. STING (Jin et al., 2011) and Ifnar1 (Hwang et al., 1995) knockout strains have been described previously. STING+/− and Ifnar1+/− mice on the congenic C57/BL6 background were crossed with TDP-43T/+ mice to create TDP-43T/+STING+/− or TDP-43T/+Ifnar1+/−, respectively, and each further crossed with other non-littermate mice of the same genotype in order to generate the offspring of TDP-43T/+STING+/+, TDP-43T/+STING+/−, TDP-43T/+STING−/−, TDP-43T/+Ifnar1+/− and TDP-43T/+Ifnar1−/−. Care of the TDP-43 mice was adapted from previously described methods (Hwang et al., 1995). Mice were genotyped using primers as follows: hsA315T FW, 5′-GGA TGA GCT GCG GGA GTT CT-3′ (SEQ ID NO:9); hsA315T RV, 5′-TGC CCA TCA TAC CCC AAC TG-3′ (SEQ ID NO:10); mmSTING FW, 5′-GCT GGG AAT TGA ACG TAG GA-3′ (SEQ ID NO:11); mmSTING RV, 5′-GAG GAG ACA AAG GCA AGC AC-3′ (SEQ ID NO: 12); mmSTING KO FW, 5′-GTG CCC AGT CAT AGC CGA AT-3′ (SEQ ID NO:13); mmIfnar1 FW, 5′-CGG AGA ACC TGC GTG CAA TC-3′ (SEQ ID NO:14); mmIfnar1 RV, 5′-TCC CGG ACA AGA CGG GAA CAT GTG G-3′ (SEQ ID NO: 15); mmIfnar1 KO FW, 5′-CGG AGA ACC TGC GTG CAA TC-3′ (SEQ ID NO:16). All mice were given DietGel Boost (ClearH2O) in a cup on the floor of the cage from P30 until the experiment endpoint to ensure that the impaired mice could easily access their food and water. The mice were weighed and visually checked for an ALS phenotype by animal technicians daily until they reached the euthanasia end point of severe motor dysfunction (see gait impairment scoring). Animal procedures were approved by the Walter and Eliza Hall Institute Animal Ethics Committee.

### Phenotype Scoring and Motor Assessment

The inventors collected data for gait impairment and other motor phenotypes in each mouse line using adapted or previously described methods (Hwang et al., 1995; Samson et al., 2015; Wang et al., 2014).

Gait impairment scoring. TDP-43 mice exhibit abnormal motor control characteristic swimming gait approximately P80, scoring was performed blinded to the genotype twice a week by animal technicians until the humane euthanasia end point or P300. Taken briefly, a score of 0 was given to the mouse with no motor impairment; a score of 1 was given to the mouse with a tremor while walking; a score of 2 was given to the mouse displaying a lowered pelvis and swimming gait while moving forward; a score of 3 was given to the mouse struggling to move forward and dragging its abdomen on the ground; a score of 4 marked the euthanasia end point in which the mouse failed to upright itself within 30 s. The scoring was interpreted for the slope of the linear regression across the lifespan per mouse, indicating progression of ALS-associated motor dysfunction.

The Rotarod test. Motor co-ordination and balance was measured using a rotating rod (Rotamex-5, Columbus Instruments). It measured the time (latency) it takes the mouse to fall off the apparatus accelerating from 4 to 40 rpm in 288 s (1 rpm/8 s). All mice at P120-130 received a 3-day training with three trials a day prior the assay. On the day of testing, mice were kept in their cages and acclimatised to the procedure room for at least 15 minutes. The test phase consists of three assays separated by 15-minute intervals to avoid habituation, and the average of three assays was taken into data analysis.

Open Field (OF) test. To quantify differences in generalised locomotor activity, mice were subjected to the OF test as described (Samson et al., 2015). In brief, mice at P120-130 were placed in the centre of a custom-built circular arena with a white melamine floor (diameter: 90 cm) and black plastic wall (height: 39 cm). This was performed in a quite (˜50 decibels) and dimly lit (˜35 lux) room. The OF was wiped clean with 70% ethanol and allowed to dry between test sessions to minimise olfactory cues. The movement of each mouse in the OF was video captured using an overhead HD C615 webcam (Logitech) and then a detailed analysis of movement was performed using the open source ImageJ (National Institutes of Health, USA) and MouseMove. The ‘distance travelled’ and ‘fractional time spent stationary’ were chosen as the most relevant to measuring the locomotor differences between unaffected and ALS-affected mice.

### Mouse CNS Tissue Collection

Brains and spinal cords were collected from mice following cardiac perfusion with PBS. For cytokine profiling, tissues were homogenised with metal beads at 30 Hz for 90 seconds in 1 mL Trizol (ThermoFisher Scientific) using TissueLyser II (Qiagen) then total RNA was isolated for qPCR. For immunochemistry, mice were perfused with 4% paraformaldehyde (PFA) after perfusion with PBS. Tissues were then immersed in 4% PFA for 3 days, cryoprotected and embedded for cryosection (7 μm). Cresyl violet was used to stain nissl bodies as a marker to compare neuronal density in cortical layer V (neurons/mm2).

### Western Blot Analysis

Cells were lysed in 1×RIPA buffer (20 mM Tris-HCl pH 7.4, 150 mM NaCl, 1 mM EDTA, 1% Triton X-100, 10% glycerol, 0.1% SDS, 0.5% deoxycholate, 10 mM NaPPi, 5 mM NaF and 1 mM Na3VO4) supplemented with 1 mM PMSF and cOmplete protease inhibitors (Roche Biochemicals), processed through Pierce centrifuge columns (Thermo Scientific) to remove DNA and denatured at 95° C. for 10 min. Samples were separated on Novex 4-12% precast SDS-PAGE gels (Thermo Scientific) with MES running buffer (Thermo Scientific), and subsequently transferred onto polyvinylidene difluoride (PVDF) membrane (Millipore). Membranes were blocked in 5% skim milk in Tris-buffered saline (TBS) containing 0.1% Tween 20 (Sigma) before overnight incubation with specific primary antibodies at 4° C.: anti-P-TBK1 Ser172 (CST; clone D52C2, 8483), anti-TBK1 (CST; D1B4, clone 3504), anti-P-IRF-3 Ser396 (CST; clone D6O1M, 29047), anti-IRF-3 (CST; clone D83B9, 4302), anti-P-p65 Ser536 (CST; clone 93H1, 3033), anti-p65 (CST; clone C22B4, 4764), anti-TFAM (CST; clone D5C8, 8076), anti-STING (CST; clone D2P2F, 13647), anti-cleaved caspase-3 Asp175 (CST; clone 5A1E, 9664), anti-GFP (Thermo Scientific; A-11122), anti-cyclophilin F (abcam; clone EPR11311-121, ab231155), anti-FLAG (In-house; clone 9H1, 1:2000) or anti-ß-Actin-HRP (Santa Cruz Biotech, clone C4, sc-47778; 1:5000). All listed primary antibodies were used at 1:1000 unless clarified. Membranes were then washed and incubated with appropriate RP-conjugated secondary antibodies, developed immunoreactivity (Chemiluminescent HRP substrate, Millipore) and imaged using the ChemiDoc Touch Imaging System (BioRad).

TDP-43 mtDNA Immunoprecipitation

Following induction of TDP-43 for the time indicated, approximately 10×106 HEK293T or MEFs were lysed at 4° C. for 30 min with 1 mL of 1% NP-40 buffer (1% NP-40, 20 mM Tris-HCl pH 7.4, 150 mM NaCl, 1 mM EGTA, 10% glycerol, 10 mM NaPPi, 5 mM NaF and 1 mM Na3VO4), supplemented with 1 mM PMSF and cOmplete protease inhibitors (Roche Biochemicals). Immunoprecipitation of FLAG-tagged hTDP-43 or mouse cGAS was performed using anti-FLAG M2 affinity Gel (Sigma). Samples were incubated at 4° C. for 2 hours on a rotator, washed extensively and then subjected to DNA extraction using a NucleoSpin Tissue XS kit (Macherey-NaGel). Coprecipitated DNA was analysed by real-time PCR as described (White et al., 2014).

### Quantitative Real-Time PCR

Total RNA was isolated using the ISOLATE II RNA Mini Kit (Bioline) as per manufacturers' instructions and reverse transcribed using oligo(dT) nucleotides. Primers for cytokines, mitochondrial DNA and housekeeping genes were used as described previously (White et al., 2014; De Nardo et al., 2014). Others were designed using the Integrated DNA Technologies online tool as follows: hsIFNB1 FW, 5′-TGT CGC CTA CTA CCT GTT GTG C-3′ (SEQ ID NO:17); hsIFNB1 RV, 5′-AAC TGC AAC CTT TCG AAG CC-3′ (SEQ ID NO:18); mmIfnb1 FW: 5′-CCA GCT CCA AGA AAG GAC GA-3′ (SEQ ID NO:19); mmIfnb1 RV: 5′-TGG ATG GCA AAG GCA GTG TA-3′ (SEQ ID NO:20); mmIfna6 FW: 5′-GCT TTC CTG ATG GTT TTG GTG-3′ (SEQ ID NO:21); mmIfna6 RV: 5′-AGG CTT TCT TGT TCC TGA GG-3′ (SEQ ID NO:22). Quantitative real-time PCR was performed using SYBR Green/ROX qPCR Master Mix (Thermo Scientific) on a ViiA 7 Real-Time PCR system (Thermo Scientific).

cGAMP Competitive ELISA

Levels of cGAMP in cell lysates, murine serum and CNS tissues from TDP-43T/+ mice and ALS patients were measured by 2′3′-cGAMP competitive ELISA Kit (Cayman Chemical, 501700). For sample preparation, cells were lysed in M-PER™ Mammalian Protein Extraction Reagent (Thermo Scientific) whereas CNS tissues were homogenised in 80% methanol, centrifuged at 21,000×g for 5 minutes at 4° C. to remove debris and the soluble fraction freeze-dried before resuspension in PBS. Approval to use post-mortem human tissue was granted by the University of Melbourne Human Ethics Committee (approval numbers 1238124 and 1750665) and all tissues obtained from the Victorian Brain Bank.

### Data Representation and Statistical Analyses

Cytokine expression from qPCR is represented as gene expression relative to Hprt expression as δCt method. Where indicated, relative gene expression in TDP-43 WT or mutant overexpressing cells was further normalised to expression in vector control transfected/overexpressed cells of the same genotype in the same experiment and represented as fold change. Data are typically mean±s.e.m and analysed by t-test between two groups or one- or two-way ANOVA followed by a Sidak or Dunnett multiple comparison test as appropriate. GraphPad Prism 7 was used for all charts and statistical analyses. A P values <0.05 was considered as significant.

### Example 2—Inflammatory Signalling from TDP-43 is Dependent on cGAS/STING

The overexpression of wild-type or mutant TDP-43 in human cell lines results in activation of NF-kB and IFN pathways. Only a select few cytoplasmic innate immune sensors can trigger both of these inflammatory pathways, and so a panel of mouse embryonic fibroblast (MEF) cell lines deficient in these candidates were tested for responses to inducible expression of wild-type or mutant TDP-43.

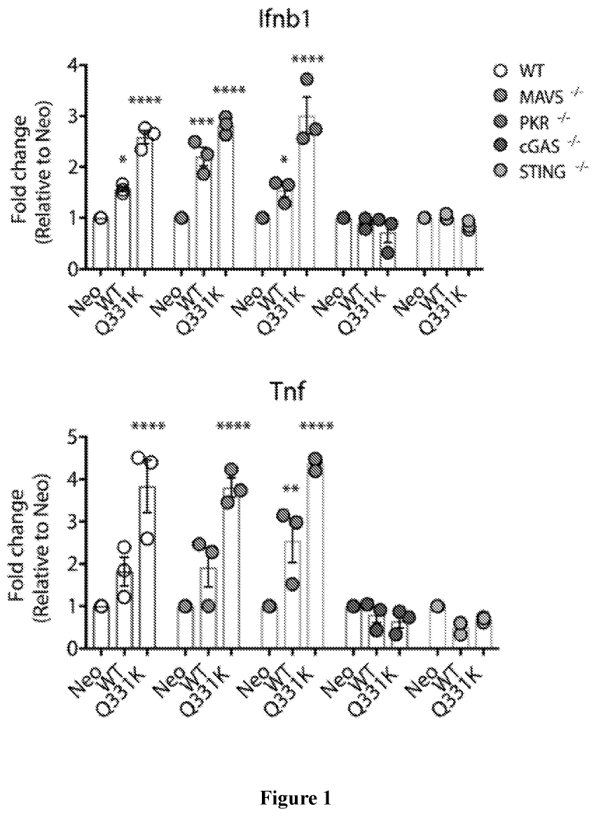

Cells were analysed 72 hours after TDP-43 induction, before the initiation of cell death, to avoid indirect activation of immune sensors. As TDP-43 is an RNA binding protein, sensors of cytoplasmic RNA were interrogated first, including RIG-I or MDA-5 (via the deletion of the conserved signalling adaptor MAVS) and PKR (FIG. 1). Surprisingly, none of these innate immune sensors reduced NF-kB or IFN activation downstream of TDP-43, and instead, deletion of cGAS, a sensor of cytoplasmic DNA, returned these pathways to baseline (FIG. 1). cGAS signals via a second messenger, cGAMP, to trigger STING. To confirm these findings, genetic deletion of STING was tested. FIG. 1 shows that genetic deletion of STING also prevents TDP-43 induced inflammation.

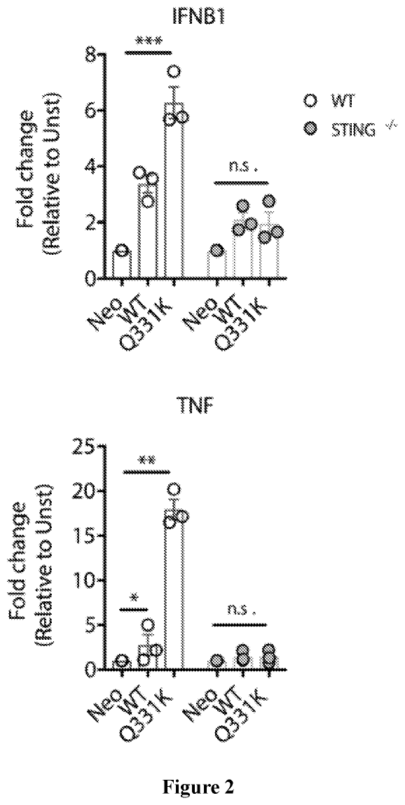

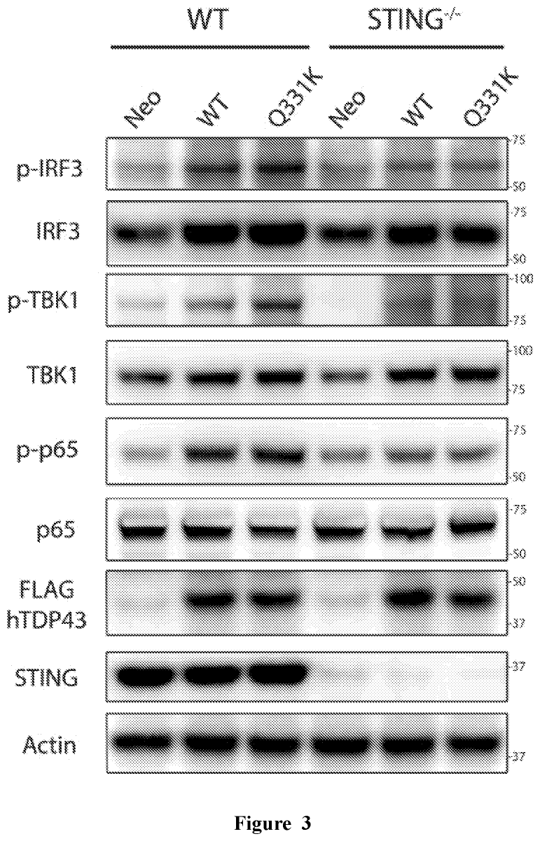

As these results come from a mouse fibroblast cell line, the findings were confirmed in human myeloid Thp1 cells, with CRISPR mediated deletion of STING. In this human cell line IFN and NF-KB pathways are significantly reduced by deletion of STING, as demonstrated by cytokine production (FIG. 2) and analysis of the signalling pathways via western blot (FIG. 3).

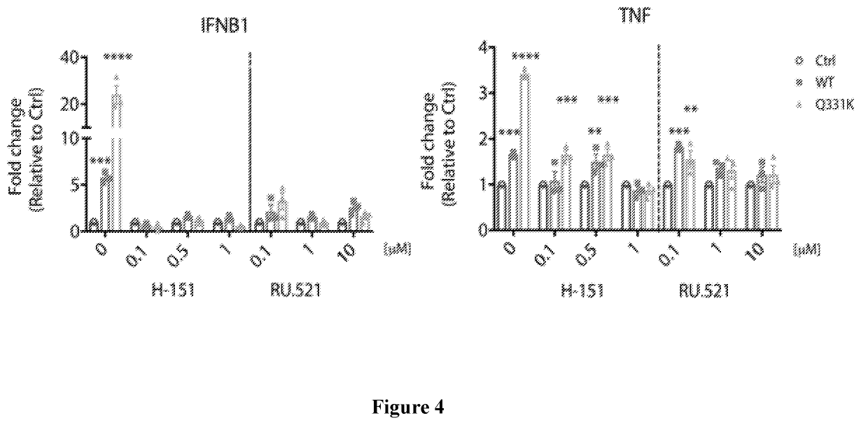

Finally, to determine if pharmacological blockade of the cGAS-STING pathway is feasible, recently described inhibitors of cGAS (RU.521, described in Vincent et al., 2017) and STING (H-151 described in Haag et al) were tested. Indeed, these drugs prevented the production of IFN and TNF in response to overexpressed wild-type and mutant TDP-43 (FIG. 4). These results identify cGAS as the immune sensor regulating neuroinflammation associated with TDP-43 and further demonstrate the effectiveness of reducing neuroinflammation in TDP-43 proteinopathies by administering a cGAS-STING pathway inhibitor.

### Example 3—TDP-43 Triggers mtDNA Release into the Cytoplasm

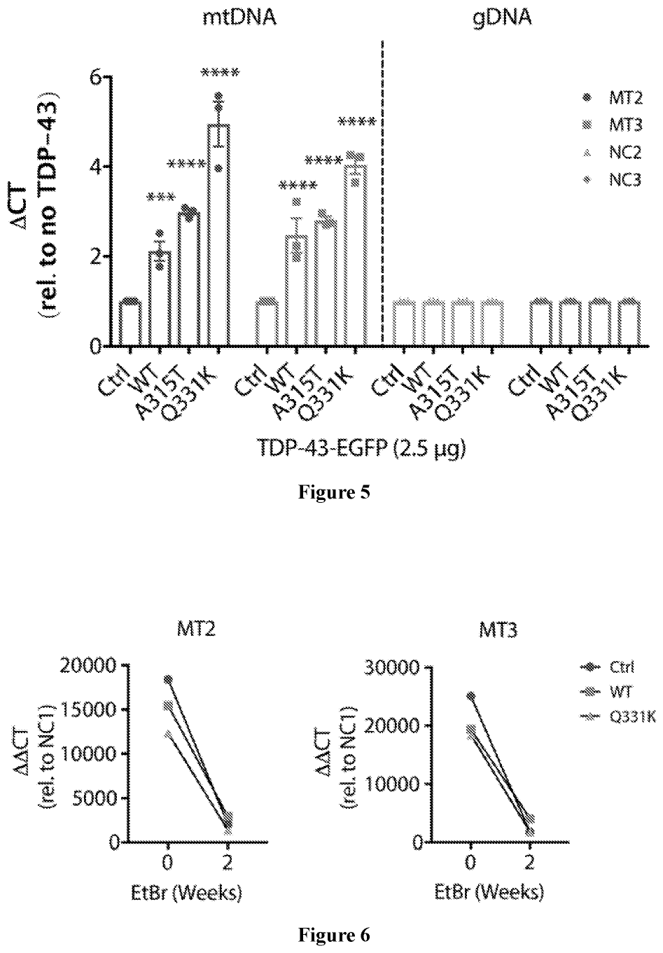

In sterile settings, cGAS is known to respond to dsDNA of mitochondrial or nuclear origin. To ascertain the source of DNA activating cGAS in response to TDP-43, FLAG-tagged cGAS was immunoprecipiated from cells overexpressing wild-type or mutant TDP-43, and qPCR for mitochondrial (Nd2 and Nd5) or nuclear (POLG) genes (White et al., 2014) was directly performed on the precipitate. This showed that in response to TDP-43, cGAS was bound to mitochondrial, and not nuclear DNA (FIG. 5).

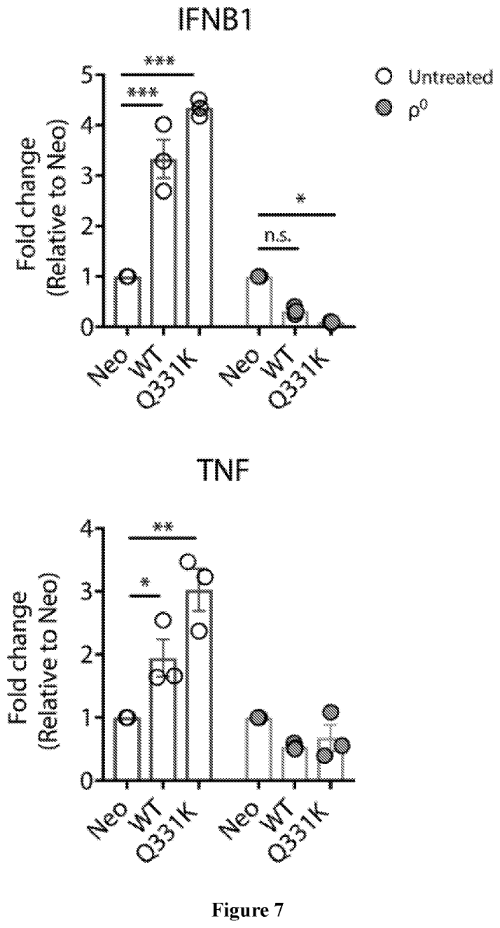

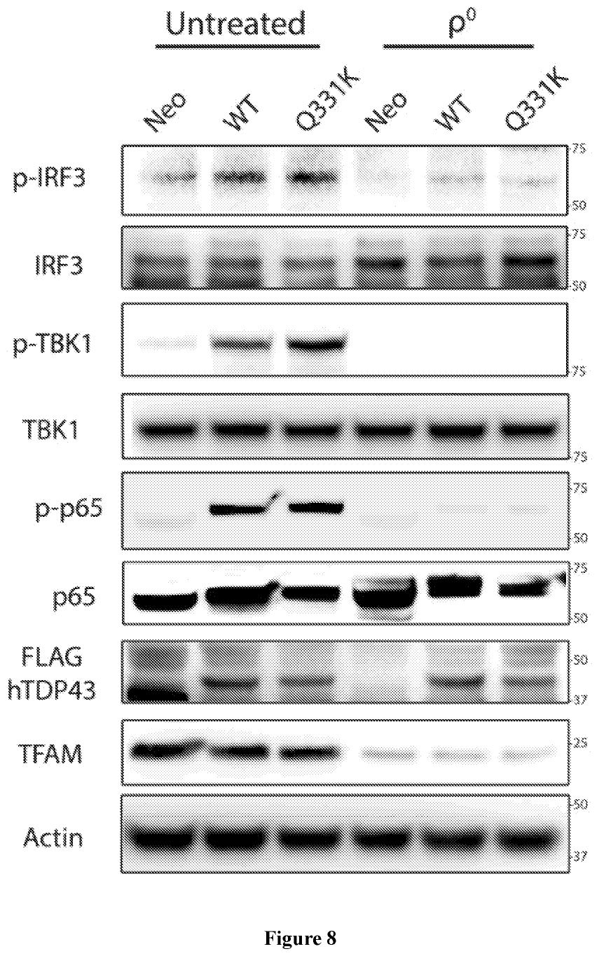

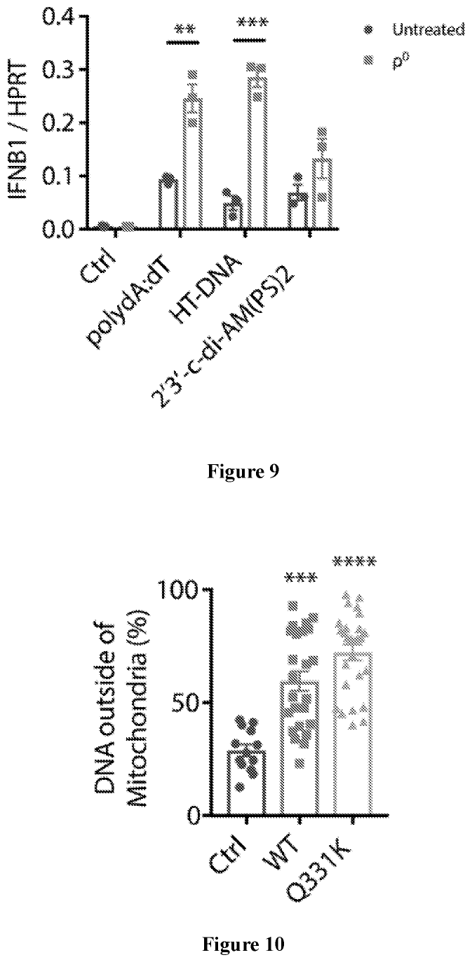

It is also possible to make cell lines deficient for mitochondrial DNA by culturing them with lose dose ethidium bromide. Accordingly, the Thp1 model overexpressing WT and mutant TDP-43 was exposed to low doses of ethidium bromide and were referred to as ρ⁰ (FIG. 6). Inflammatory cytokine production (FIG. 7) and activation of cGAS-STING signaling pathways (FIG. 8) were then quantified. Inflammatory cytokine production and activation of downstream signaling all returned to baseline in the ρ⁰ cells depleted of mitochondrial DNA. There was no intrinsic functional abnormality in the cGAS/STING signaling capability of these cells, as demonstrated by the induction of appropriate responses to a STING ligand (FIG. 9). As a final confirmation, cells were imaged, which showed leakage of mitochondrial DNA into the cytoplasm in response to TDP-43, which was further augmented by mutant TDP-43 (FIG. 10).

### Example 4—TDP-43 Triggers mtDNA Release into the Cytoplasm Via the mPTP

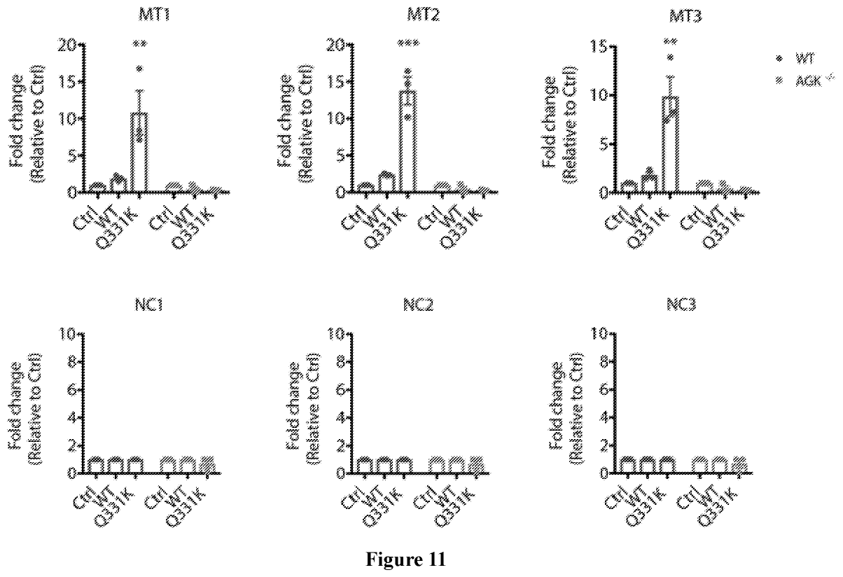

Previously, it has been reported that TDP-43 gains access to the mitochondrial matrix via TIM22 (Wang et al., 2016). This was confirmed by the finding that TDP-43 (WT or mutant) did not bind mitochondrial DNA in cells where TIM22 is non-functional (FIG. 11).

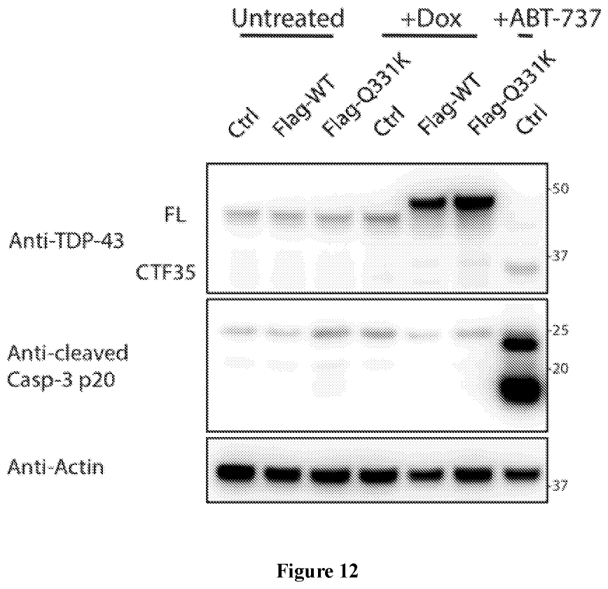

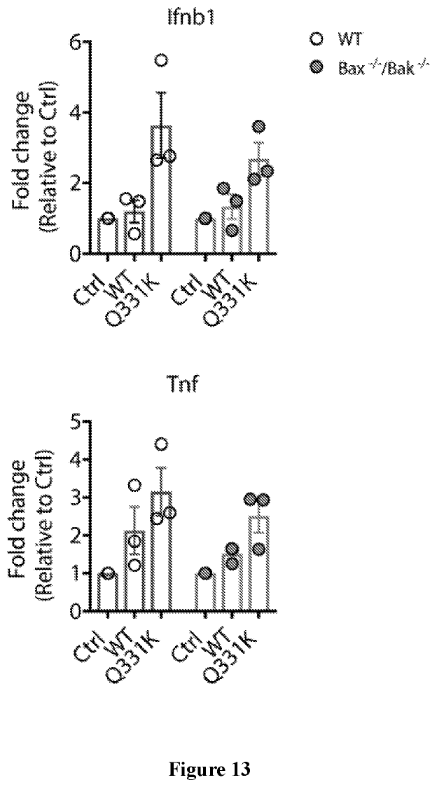

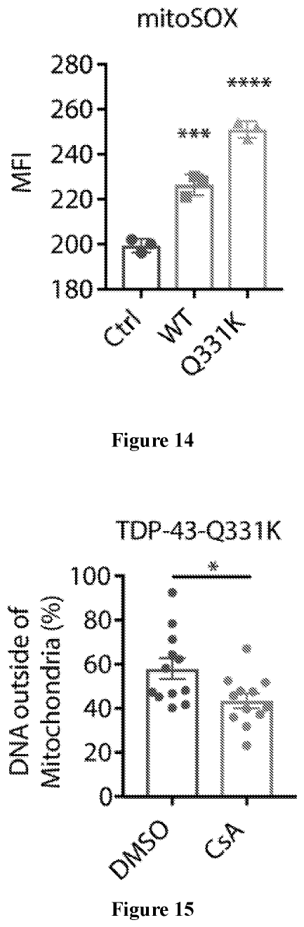

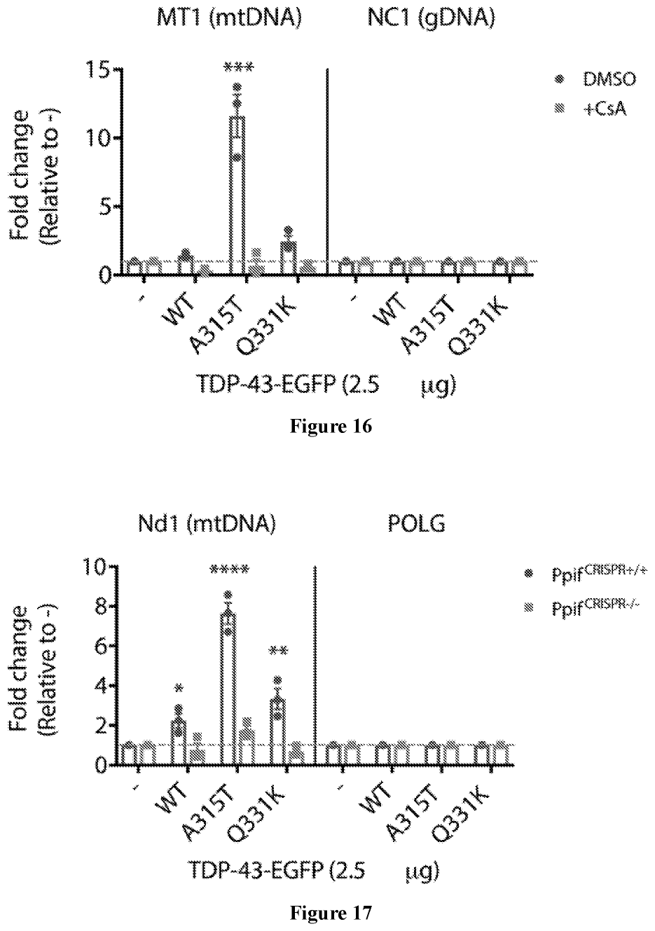

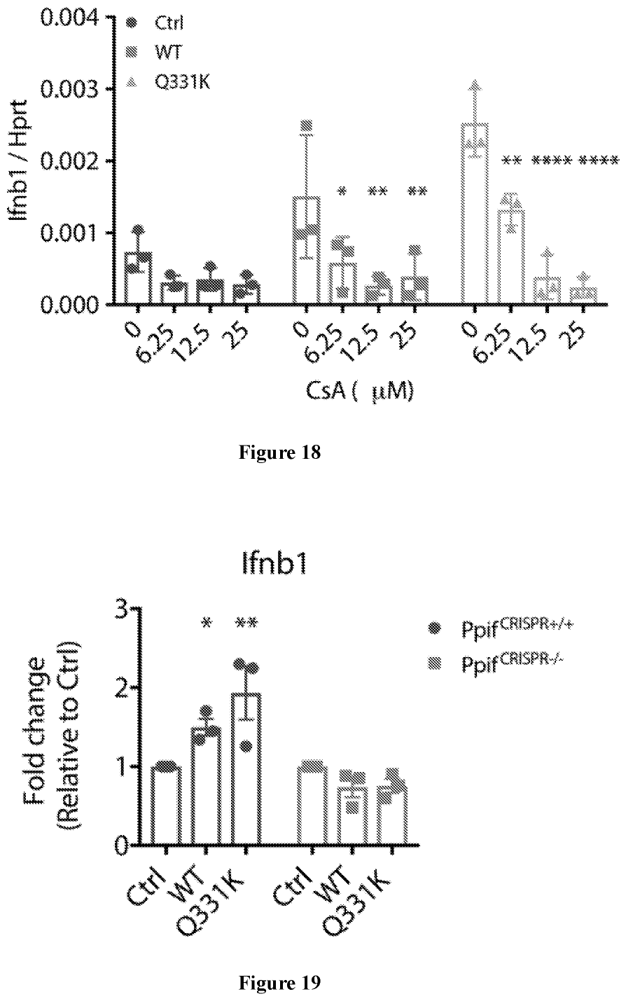

It has also been reported that TDP-43 is capable of inducing apoptosis under certain conditions, which could trigger Bak/Bax permeabilization of the outer mitochondrial membrane and leakage of DNA in the cytoplasm. However, at the timepoints studied in the assays shown in FIG. 12 (cleaved caspase-3), there is no evidence of apoptosis. Furthermore, the deletion of Bak/Bax had no effect on inflammatory cytokine production in response to wild-type or mutant TDP-43 (FIG. 13). Instead, other markers of mitochondrial destabilisation such as upregulation of ROS were observed (FIG. 14), which are thought to destabilize and thus open the mitochondrial Permeability Transition Pore (mPTP) (Nguyen et al., 2011). In agreement with this, pharmacological inactivation of the mPTP using cyclosporin A (CsA) prevented mtDNA leakage into the cytoplasm (FIG. 15). Additionally, inhibition of the mPTP (CsA) or CRISPR mediated genetic deletion of the critical mPTP component cyclophilin D (CypD) prevented mtDNA binding to cGAS (FIGS. 16 and 17 respectively) and inflammation in response to TDP-43 (FIGS. 18 and 19 respectively). These data demonstrate the mechanism by which TDP-43 can mislocalise into mitochondria, opening the mPTP and resulting in mtDNA leakage into the cytoplasm, thereby activating the cGAS-STING pathway.

### Example 5—Genetic Deletion of STING Prevents Disease in an ALS Mouse Model

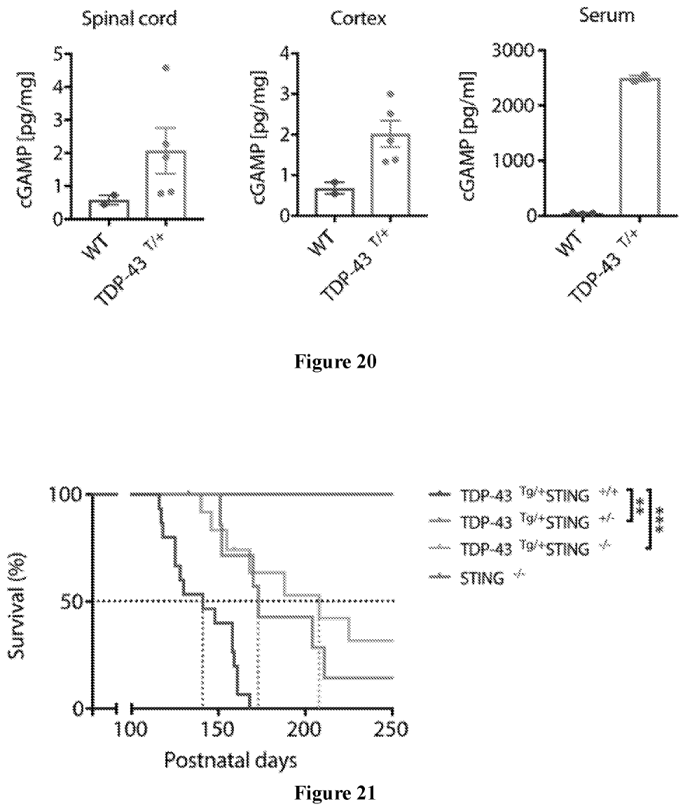

To establish if the cGAS-STING pathway is responsible for neurodegeneration in response to TDP-43 in vivo, a well described model of ALS and FTLD with human TDP-43 (A315T) overexpression in mice was used (Wils et al., 2010). When placed on a jellified diet, this strain avoids early lethality due to gastrointestinal blockage, and succumbs to symptoms of motor neuron degeneration around 140 days of age. Critically, elevated levels of the messenger for cGAS-STING mediated signalling, cGAMP, were observed in the spinal cord and cortex of these mice on autopsy, and also in the circulation of mice with established disease (FIG. 20). Following this, the ALS model strain was crossed to genetically delete STING. In mice with homozygous deletion of STING there was a very significant extension of life span by 40%, to approximately 180 days (FIG. 21). Notably, deletion of only a single allele of STING also afforded significant extension of life, with survival increased to 170 days (FIG. 21), indicating the suitability of pharmacological intervention of STING for treating or preventing ALS and FTLD.

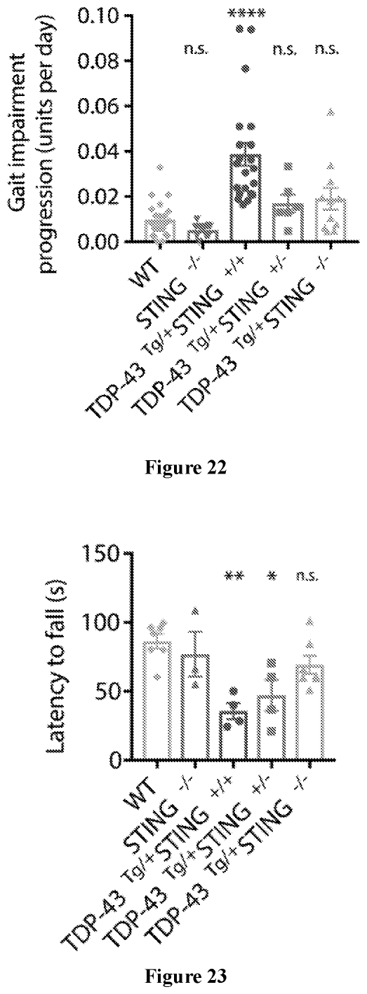

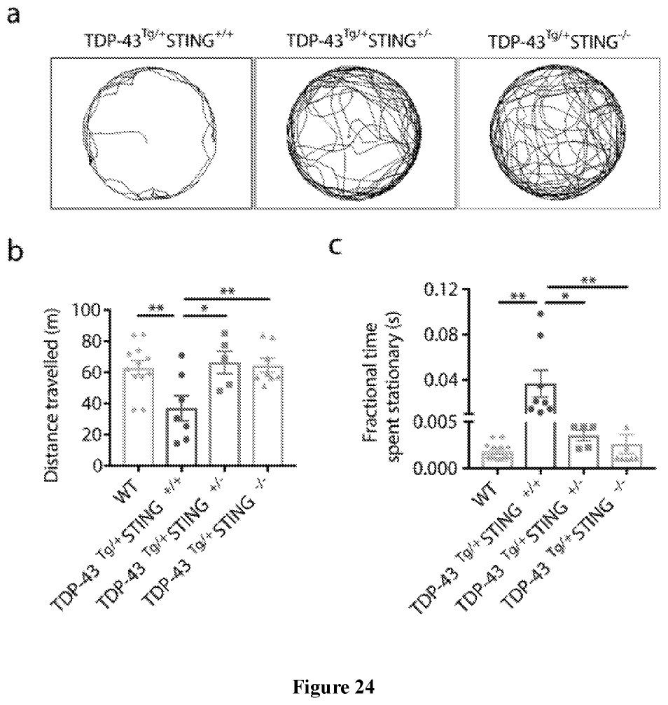

For subjects with neurodegeneration associated with TDP-43, it is essential to not only extend life, but also ameliorate disease symptoms. At day 120, TDP-43 mutant mice were unable to maintain latency in the gold standard “rotarod” test, and suffered a progressive deterioration in gait (FIGS. 22 and 23). However, mice deficient for STING were significantly ameliorated (FIGS. 22 and 23). At this timepoint the deletion of STING also significantly increased the distance travelled by TDP-43 mutant mice in an open field test, and reduced their stop fraction (FIG. 24).

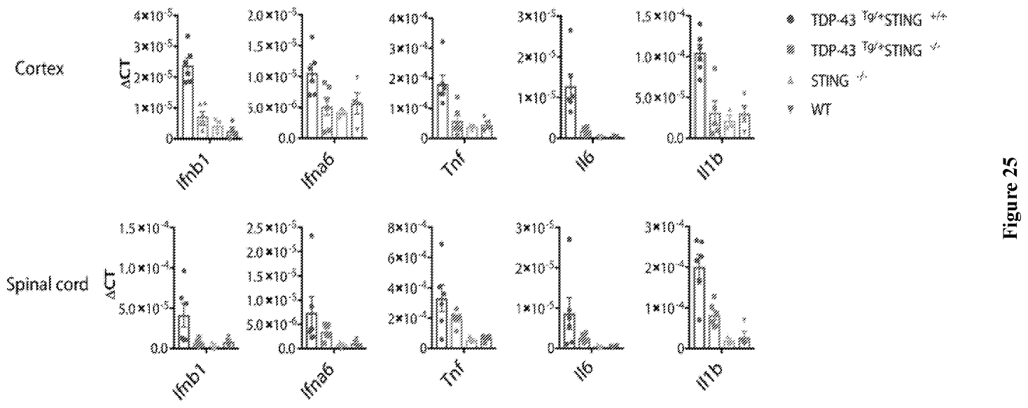

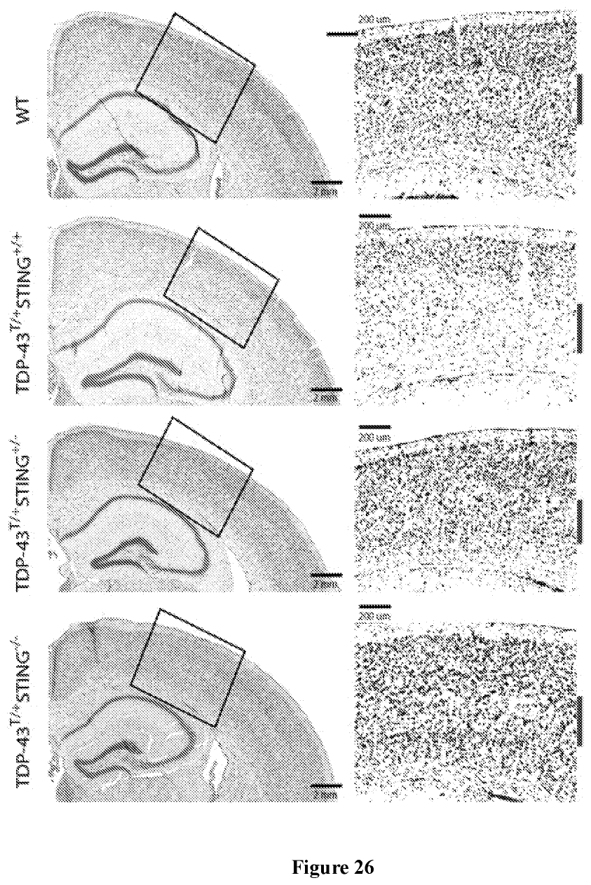

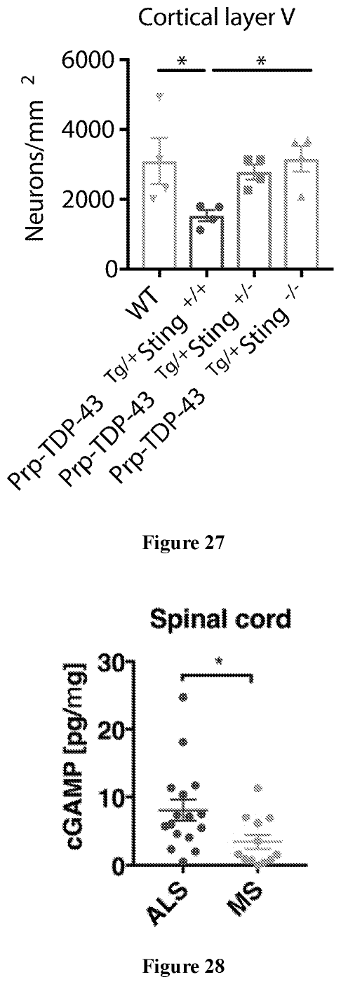

Finally, the beneficial effect of deleting STING in this mouse model of ALS and FTLD was confirmed as being associated with decreased neuroinflammation and neurodegeneration. Specifically, inflammatory cytokines were no longer upregulated in the cortex and spinal cord (FIG. 25) and there was no longer a significant loss of neurons from cortex layer 5 as quantified by nissl body staining (FIG. 26 and FIG. 27).

Therefore, these results demonstrate that deletion of STING results in a dramatic extension of life as well as reduction in symptoms for an aggressive ALS and FTLD associated mutation in mice. The observation that deletion of only one allele of STING also significantly improves disease further indicates that pharmacological inhibition of the cGAS-STING pathway is efficacious.

### Example 6—Elevated cGAMP in Spinal Cord of Patients with ALS

Finally, the levels of cGAMP were quantified in spinal cord samples from people who had ALS and compared this to samples from cases of progressive multiple sclerosis (MS) as a neurological control (Table 1). This documented a significant increase in cGAMP for the ALS samples independent of age, sex or post-mortem interval (FIG. 28). These results implicate cGAS as an important immune sensor regulating neuroinflammation associated with TDP-43 in ALS.

TABLE 1
Demographic characteristics of the patients.

| | |
|---|---|
| Amyotrophic Lateral | |
| Sclerosis | Multiple Sclerosis |
| (N = 16) | (N = 12) |

Age - yr

| | | |
|---|---|---|
| Median | 66.2 | 66.5 |
| Range | 53.6-79.9 | 38.5-74.5 |

Sex - no. (%)

| | | | |
|---|---|---|---|
| Male | 13 | (81.3) | 7 (58.3) |
| Female | 3 | (18.8) | 5 (41.7) |

Post-Mortem Interval - hrs

| | | |
|---|---|---|
| Median | 28.5 | 38.25 |
| Range | 7-56 | 22-62 |

ALS symptoms - no. (%)

| | | |
|---|---|---|
| Bulbar | 4 | (25.0) |
| Lower Limb | 9 | (56.3) |
| Upper Limb | 6 | (37.5) |

MS symptoms - no. (%)

| | |
|---|---|
| Primary Progressive | 4 (33.3) |
| Secondary Progressive | 7 (58.3) |

### Example 7—STING Inhibition to Treat Established Disease

Using the mouse model described in Example 5, the inventors investigated if the small molecule STING inhibitor H-151 could treat established disease. When disease symptoms first appeared in TDP-43 mutant mice at 120 days of age, H-151 (3.75 mM) was administered i.p. every the other day for 4 weeks.

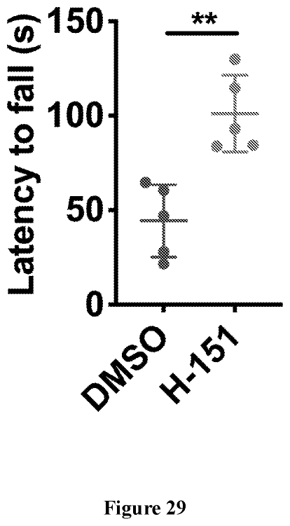

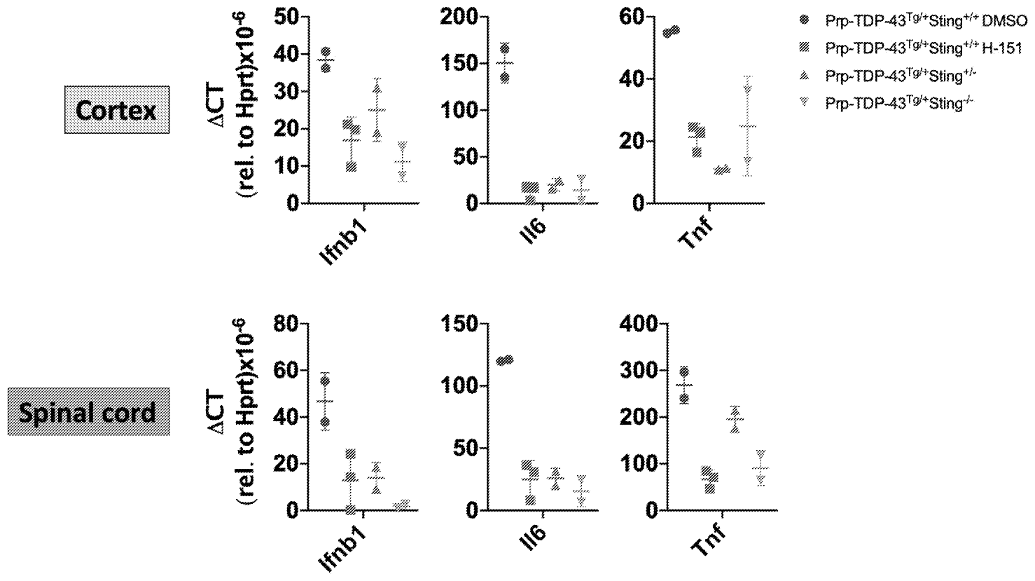

Administration of H-151 significantly improved latency to fall in a rotarod test (FIG. 29). In addition, qPCR of inflammatory gene expression in the cortex and spinal cords revealed that increased levels of IFN and NF-kB dependent cytokines is greatly reduced due to inhibition of STING using H-151 (FIG. 30). Therefore, at a molecule level, and in terms of disease symptoms, H-151 inhibition of STING could treat established disease in a mouse model of ALS and FTLD, due to TDP-43 proteinopathy.

### Example 8—Mitochondrial Damage and cGAS/Sting Activation in ALS Patient Derived iPSC Motor Neurons

Human iPSC Lines

The established human iPSC lines used in this study included two controls (NCRM-1 and NCRM-5) from the National Institute of Neurological Disorders and Stroke, one control and one ALS patient from Alessandro Rosa, one ALS patient from RIKEN and one ALS patient (TALSTDP-47.10) from the Target ALS Foundation. All iPSCs were maintained on Matrigel (Corning, 354277)-coated 6-well plates in mTeSR1 (STEMCell Technologies, 85850) containing 1× Primocin (Invivogen, ant-pm-1) and passaged 1:6 using ReLeSR (STEMCell Technologies, 05872) with ROCK inhibitor Y-27632 (STEMCell Technologies, 72304) for the first 24 hours. The medium was replaced daily. Cells were cyropreserved in mTeSR1/10% DMSO.

Generation of Motor Neuron Progenitors and iPSC-Derived Motor Neurons

Differentiation of iPSCs into ChAT⁺ motor neurons was performed as described previously (Du et al., 2015) All medium and reagents from Gibco unless otherwise stated.

Induction of Neuroepithelial Progenitors (NEPs).

Human iPSCs were cultured in a chemically defined Neural Medium: DMEM/F12:Neurobasal (1:1) supplemented with 0.5×N2, 0.5×B27, 0.1 mM L-ascorbic acid (Sigma), 1× Glutamax and 1× Primocin (Invivogen) containing 3 μM CHIR99021 (STEMCell Technologies, 72054), 2 μM Dorsomorphin (STEMCell Technologies, 72102) and 2 μM SB431542 (STEMCell Technologies, 72234) for 6 days, and the medium was changed every other day.

Induction of MNPs.

The NEPs were dissociated with ReLeSR and cultured 1:6 on Matrigel-coated plates in the Neural Medium containing 1 μM CHIR99021, 2 μM Dorsomorphin and 2 μM SB431542 for 6 days. Y-27632 was used for the first 24 hours. The medium was changed every other day. At this stage of differentiation, MNPs were either expanded in the Neural Medium containing 3 μM CHIR99021, 2 μM Dorsomorphin, 2 μM SB431542, 0.1 μM all-trans retinoic acid (Sigma, R2500), 0.5 μM Purmorphamine (STEMCell Technologies, 72204) and 0.5 mM Valporic Acid (STEMCell Technologies, 72292) prior to MN diffrenertiation or cryopreserved in DMEM/F12 containing 10% FBS and 10% DMSO.

Differentiation of MN.

The MNPs were dissociated with ReLeSR and cultured on Matrigel-coated plates in the Neural Medium containing 0.5 μM all-trans RA and 0.1 μM Pur for 6 days into premature MNX1⁺ MN. Y-27632 was used for the first 24 hours and the medium was replaced every other day. Subsequently, cells were detached with Accutase (Merck Millipore, SCR005) to generate single cell suspension and matured in the medium supplemented with 0.1 μM Compound E (STEMCell Technologies, 73954) for 10 days into ChAT⁺ MNs. The medium was replaced every other day. Cells were plated in Matrigel-coated 8-well chamber slides (iBidi, 80826) to confirm cellular markers of MNs, including MNX1 (Merck Millipore, ABN174) and ChAT (Merck Millipore, ABN174) along with βIII-Tubulin (Promega, clone 5G8, G7121) staining using an inverted SP8 confocal microscopy (Leica).

### Results

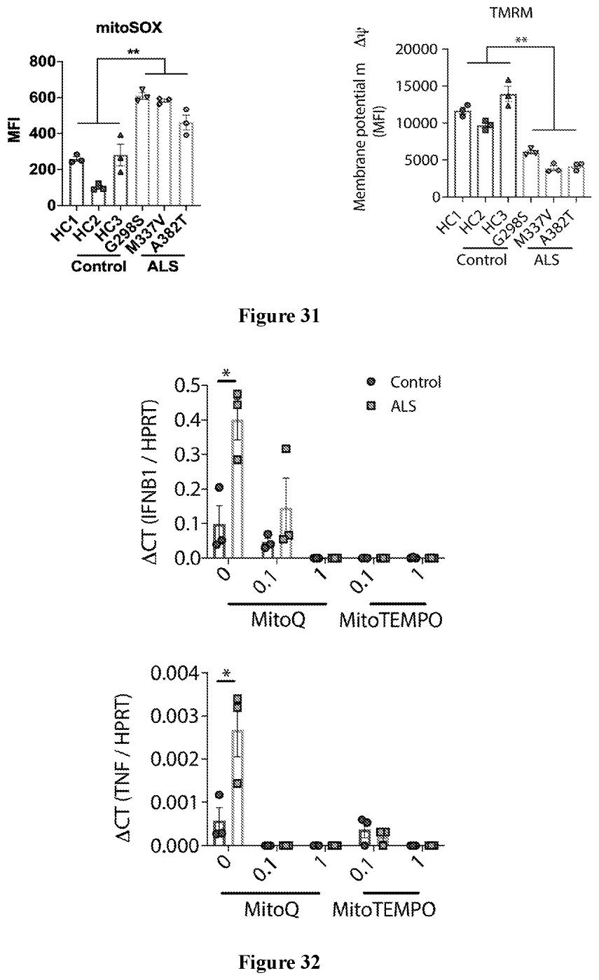

Using the iPSC derived MN mitochondrial damage and activation of the cGAS/STING pathway was confirmed. Specifically, mitochondrial destabilization markers a) mitoSOX red and b) Tetramethylrhodamine Methyl Ester (TMRM) were quantified by Flow cytometry (FACS) in iPSC-derived motor neurons. These demonstrated upregulation of ROS and loss of membrane potential (mΔψ) in ALS patient iPSC-derived motor neurons (FIG. 31). Additionally, the ROS inhibitors MitoQ and MitoTEMPO prevented IFNB1 and TNF gene induction in human iPSC-derived motor neurons from healthy controls and ALS patients carrying mutations in TARDBP (TDP-43), as shown in FIG. 32.

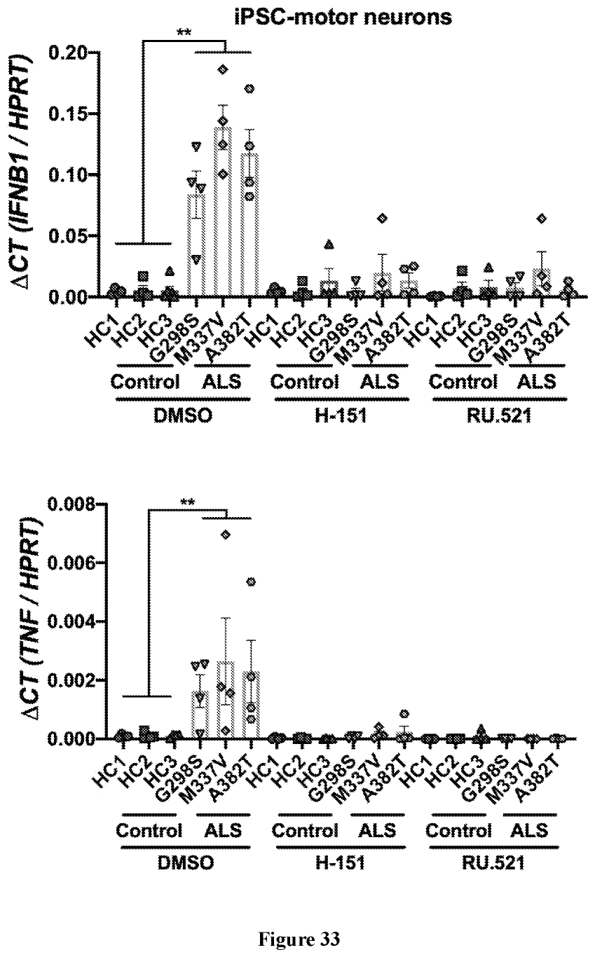

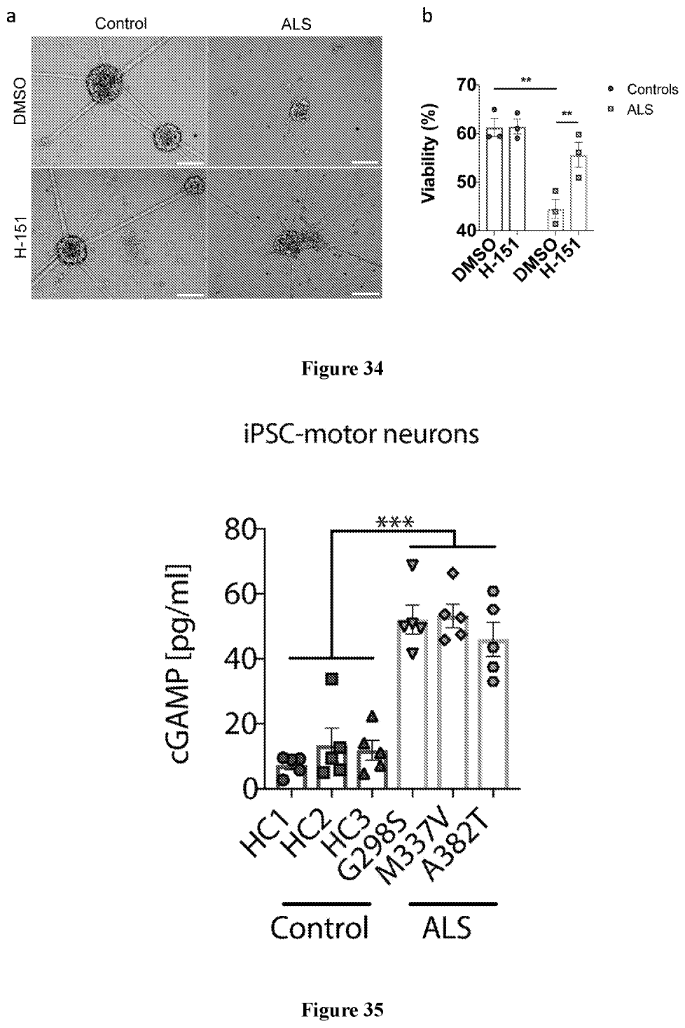

To implicate a role of the cGAS/Sting pathway, the cGAS inhibitor RU.521 and STING inhibitor H-151 were tested, and it was found that they prevent IFNB1 and TNF gene induction in human iPSC-derived motor neurons from healthy controls and ALS patients carrying mutations in TARDBP (TDP-43), as shown in FIG. 33. Moreover, when the motor neurons are left for 4 weeks in culture, cell death is observed over this period of time, however inhibition of STING mitigated ALS-associated cytotoxicity. This was demonstrated by imaging of the cells (FIG. 34a) and measured by LDH as a readout of cell number (FIG. 34b). The signalling metabolite cGAMP is proposed as a biomarker for cGAS activation in ALS, and was demonstrated to be elevated in the 3 ALS patient derived motor neurons compared to 3 healthy controls (FIG. 35).

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

This application claims priority from Australian Patent Application No 2018904175 filed on 2 Nov. 2018 and entitled “Methods of treatment, prevention and diagnosis”. The entire contents of that application are hereby incorporated by reference.

All publications cited herein are hereby incorporated by reference in their entirety. Where reference is made to a URL or other such identifier or address, it is understood that such identifiers can change and particular information on the internet can come and go, but equivalent information can be found by searching the internet. Reference thereto evidences the availability and public dissemination of such information.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

### REFERENCES

Ablasser et al. (2013) Nature 503:530-534
Armakola et al. (2012) Nat Genet 44, 1302-1309
Baker and Masters (2018) Methods Mol Biol 1714, 41-55
Constantini et al. (2008) Cancer Biotherm Radiopharm 23: 3-24
De Coupade et al. (2005), Biochem J, 390:407-418
De Nardo et al. (2014) Nat Immunol 15, 152-160
Deshayes et al. (2008) Adv Drug Deliv Rev 60:537-47
Du et al. (2015) Nat Commun. 6:6626
Gao et al. (2013) Cell 153:1094-1107
Haag et al. (2018) Nature 559:269-273
Hashiguchi and Zhang-Akiyama (2009) Methods Mol Biol 554, 383-391
Hall et al. (2017) PLoS ONE 12:e0184843
Howl et al. (2007), Biochem Soc Trans 35:767-769
Hwang et al. (1995) Proc Natl Acad Sci USA 92, 11284-11288
Irving et al. (2012) Immunity 36, 795-806
Jin et al. (2011) J Immunol 187, 2595-2601
Lee et al. (2012) Nature Reviews Neuroscience 13:38-50
Kabashi et al. (2008) Nat Genet 40:572-574
Koppelhus et al. (2008) Bioconj Chem 19:1526-34
Largaespada et al. (1996) J Immunol Methods 197:85-95
McArthur et al. (2018) Science 359
Nguyen et al. (2011) J Biol Chem 286:40184-40192
Nguyen et al. (2017) Immunity 47, 498-509 e496
Rutherford et al. (2008 PLoS Genet 4:e1000193
Samson et al. (2015) Sci Rep 5, 16171
Seeds and Miller (2011) Curr Protoc Immunol, February; Chapter 14:Unit14.21.1-11
Sreedharan et al. (2008) Science 319:1668-1672
Swarup et al. (2011) J Exp Med 208:2429-2447
Vincent et al. (2017) Nat Commun 8, 750
Walker et al. (2013) PLoS One 8:e81170
Walzer et al. (2007) Proc Natl Acad Sci USA 104:3384-3389
Wang et al. (2011) Glia 59:946-958
Wang et al. (2016) Nat Med 22:869-878
Wegorzewska et al. (2009) Proc Natl Acad Sci USA 106, 18809-18814
White et al. (2014) Cell 159:1549-1562
Wils et al. (2010) Proc Natl Acad Sci USA 107:3858-3863

SEQUENCE LISTING

<110> The Walter and Eliza Hall Institute of Medical Research
<120> METHODS OF TREATMENT, PREVENTION AND DIAGNOSIS
<130> 529034PCT
<150> AU 2018904175
<151> 2018-11-02
<160> 22
<170> PatentIn version 3.5
<210> 1
<211> 24
<212> RNA
<213> Artificial Sequence
<220>
<223> CRISPR guide sequence
<400> 1

| | |
|---|---|
| ucccagagca cacucuccgg uacc | 24 |
| <210> 2 | |
| <211> 24 | |
| <212> RNA | |
| <213> Artificial Sequence | |
| <220> | |
| <223> CRISPR guide sequence | |
| <400> 2 | |
| aaacgguacc ggagagugug cucu | 24 |
| <210> 3 | |
| <211> 24 | |
| <212> RNA | |
| <213> Artificial Sequence | |
| <220> | |
| <223> CRISPR guide sequence | |
| <400> 3 | |
| uccccgugcc aaagacugca ggua | 24 |
| <210> 4 | |
| <211> 24 | |
| <212> RNA | |
| <213> Artificial Sequence | |
| <220> | |
| <223> CRISPR guide sequence | |
| <400> 4 | |
| aaacuaccug cagucuuugg cacg | 24 |
| <210> 5 | |
| <211> 36 | |
| <212> DNA | |
| <213> Artificial Sequence | |
| <220> | |
| <223> Mutagenesis oligonucleotide | |
| <400> 5 | |
| ggattaatgc tgaacgtacc aaagttcatc ccacca | 36 |
| <210> 6 | |
| <211> 36 | |
| <212> DNA | |
| <213> Artificial Sequence | |
| <220> | |
| <223> Mutagenesis oligonucleotide | |
| <400> 6 | |
| tggtgggatg aactttggta cgttcagcat taatcc | 36 |
| <210> 7 | |
| <211> 29 | |
| <212> DNA | |
| <213> Artificial Sequence | |
| <220> | |
| <223> Mutagenesis oligonucleotide | |
| <400> 7 | |
| ccccaactgc tctttagtgc tgcctgggc | 29 |
| <210> 8 | |
| <211> 29 | |
| <212> DNA | |
| <213> Artificial Sequence | |
| <220> | |
| <223> Mutagenesis oligonucleotide | |
| <400> 8 | |
| gcccaggcag cactaaagag cagttgggg | 29 |
| <210> 9 | |
| <211> 20 | |
| <212> DNA | |
| <213> Artificial Sequence | |
| <220> | |
| <223> Primer | |
| <400> 9 | |
| ggatgagctg cgggagttct | 20 |
| <210> 10 | |
| <211> 20 | |
| <212> DNA | |
| <213> Artificial Sequence | |
| <220> | |
| <223> Primer | |
| <400> 10 | |
| tgcccatcat accccaactg | 20 |
| <210> 11 | |
| <211> 20 | |
| <212> DNA | |
| <213> Artificial Sequence | |
| <220> | |
| <223> Primer | |
| <400> 11 | |
| gctgggaatt gaacgtagga | 20 |
| <210> 12 | |
| <211> 20 | |
| <212> DNA | |
| <213> Artificial Sequence | |
| <220> | |
| <223> Primer | |
| <400> 12 | |
| gaggagacaa aggcaagcac | 20 |
| <210> 13 | |
| <211> 20 | |
| <212> DNA | |
| <213> Artificial Sequence | |
| <220> | |
| <223> Primer | |
| <400> 13 | |
| gtgcccagtc atagccgaat | 20 |
| <210> 14 | |
| <211> 20 | |
| <212> DNA | |
| <213> Artificial Sequence | |
| <220> | |
| <223> Primer | |
| <400> 14 | |
| cggagaacct gcgtgcaatc | 20 |
| <210> 15 | |
| <211> 25 | |
| <212> DNA | |
| <213> Artificial Sequence | |
| <220> | |
| <223> Primer | |
| <400> 15 | |
| tcccggacaa gacgggaaca tgtgg | 25 |
| <210> 16 | |
| <211> 20 | |
| <212> DNA | |
| <213> Artificial Sequence | |
| <220> | |
| <223> Primer | |
| <400> 16 | |
| cggagaacct gcgtgcaatc | 20 |
| <210> 17 | |
| <211> 22 | |
| <212> DNA | |
| <213> Artificial Sequence | |
| <220> | |
| <223> Primer | |
| <400> 17 | |
| tgtcgcctac tacctgttgt gc | 22 |
| <210> 18 | |
| <211> 20 | |
| <212> DNA | |
| <213> Artificial Sequence | |
| <220> | |
| <223> Primer | |
| <400> 18 | |
| aactgcaacc tttcgaagcc | 20 |
| <210> 19 | |
| <211> 20 | |
| <212> DNA | |
| <213> Artificial Sequence | |
| <220> | |
| <223> Primer | |
| <400> 19 | |
| ccagctccaa gaaaggacga | 20 |
| <210> 20 | |
| <211> 20 | |
| <212> DNA | |
| <213> Artificial Sequence | |
| <220> | |
| <223> Primer | |
| <400> 20 | |
| tggatggcaa aggcagtgta | 20 |
| <210> 21 | |
| <211> 21 | |
| <212> DNA | |
| <213> Artificial Sequence | |
| <220> | |
| <223> Primer | |
| <400> 21 | |
| gctttcctga tggttttggt g | 21 |
| <210> 22 | |
| <211> 20 | |
| <212> DNA | |
| <213> Artificial Sequence | |
| <220> | |
| <223> Primer | |
| <400> 22 | |
| aggctttctt gttcctgagg | |

1. A method of treating or preventing a TDP-43 proteinopathy in a subject, the method comprising administering a cGAS-STING pathway inhibitor to the subject.

2. The method of claim 1, wherein TDP-43 proteinopathy is a neurodegenerative disease.

3. The method of claim 1, wherein the TDP-43 proteinopathy is amyotrophic lateral sclerosis (ALS), motor neuron disease (MND), frontotemporal dementia (FTD), frontotemporal lobar degeneration (FTLD), Alzheimer's disease, Parkinson's disease, or inclusion body myositis (IBM).

4. The method of claim 3, wherein the ALS is sporadic ALS, or the FTLD is FTLD with ubiquitin-positive inclusions (FTLD-U).

5. The method of claim 1, wherein the cGAS-STING pathway inhibitor is a STING inhibitor that binds to STING or competitively inhibits cGAMP binding to STING.

6.-8. (canceled)

9. The method of claim 1, wherein the STING inhibitor reduces the expression of STING and is an siRNA.

10. The method of claim 5, wherein the STING inhibitor is a cyclic dinucleotide.

11. The method of claim 10, wherein the cyclic dinucleotide is a cyclic purine dinucleotide.

12. The method of claim 5, wherein the STING inhibitor is a nitrofuran derivative.

13. The method of claim 5, wherein the STING inhibitor blocks palmitoylation-induced clustering of STING.

14. The method of claim 5, wherein the STING inhibitor covalently binds to STING.

15. The method of claim 14, wherein the STING inhibitor covalently binds to Cys91 of STING.

16. The method of claim 13, wherein the STING inhibitor is a compound of the following formula: 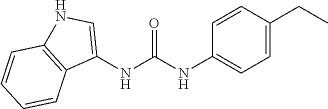 [SMILES: CCc1ccc(NC(=O)Nc2c[nH]c3ccccc23)cc1]
or a pharmaceutically acceptable salt, solvate, stereoisomer, tautomer, or prodrug thereof, diclofenac, R(−)-2,10,11-Trihydroxyaporphine hydrobromide, Dipropyldopamine hydrobromide, (±)-trans-U-50488, 2,2′-Bipyridine, SP600125, Doxazosin mesylate, Mitoxantrone, MRS 2159, Nemadipine-A, (±)-PPHT hydrochloride, SMER28, Quinine, or Quisqualic acid.

17. (canceled)

18. The method of claim 1, wherein the cGAS-STING pathway inhibitor is a cGAS inhibitor.

19. The method of claim 18, wherein the cGAS inhibitor binds to cGAS and/or inhibits cGAMP catalysis or reduces expression of cGAS.

20. The method of claim 19, wherein the cGAS inhibitor binds to the active site of cGAS.

21.-24. (canceled)

25. The method of claim 18, wherein the cGAS inhibitor is a compound of the following formula: 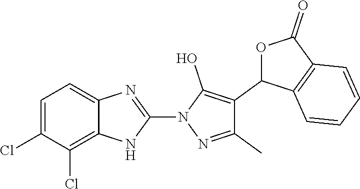 [SMILES: Cc1nn(-c2nc3ccc(Cl)c(Cl)c3[nH]2)c(O)c1C1OC(=O)c2ccccc21]
or a pharmaceutically acceptable salt, solvate, stereoisomer, tautomer, or prodrug thereof.

26. The method of claim 1, wherein the cGAS-STING pathway inhibitor is a cGAMP inhibitor.

27.-28. (canceled)

29. The method of claim 1, wherein the subject is a human.

30. The method of claim 1, wherein the cGAS-STING pathway inhibitor is administered in an amount sufficient to reduce or prevent an increase in TNFα and/or type I interferon expression.

31.-39. (canceled)


SEQUENCE LISTING (22 sequences)

SEQ ID NO: 1 - RNA, 24 nt, Artificial Sequence
  CRISPR guide sequence
ucccagagcacacucuccgguacc

SEQ ID NO: 2 - RNA, 24 nt, Artificial Sequence
  CRISPR guide sequence
aaacgguaccggagagugugcucu

SEQ ID NO: 3 - RNA, 24 nt, Artificial Sequence
  CRISPR guide sequence
uccccgugccaaagacugcaggua

SEQ ID NO: 4 - RNA, 24 nt, Artificial Sequence
  CRISPR guide sequence
aaacuaccugcagucuuuggcacg

SEQ ID NO: 5 - DNA, 36 bp, Artificial Sequence
  Mutagenesis oligonucleotide
ggattaatgctgaacgtaccaaagttcatcccacca

SEQ ID NO: 6 - DNA, 36 bp, Artificial Sequence
  Mutagenesis oligonucleotide
tggtgggatgaactttggtacgttcagcattaatcc

SEQ ID NO: 7 - DNA, 29 bp, Artificial Sequence
  Mutagenesis oligonucleotide
ccccaactgctctttagtgctgcctgggc

SEQ ID NO: 8 - DNA, 29 bp, Artificial Sequence
  Mutagenesis oligonucleotide
gcccaggcagcactaaagagcagttgggg

SEQ ID NO: 9 - DNA, 20 bp, Artificial Sequence
  Primer
ggatgagctgcgggagttct

SEQ ID NO: 10 - DNA, 20 bp, Artificial Sequence
  Primer
tgcccatcataccccaactg

SEQ ID NO: 11 - DNA, 20 bp, Artificial Sequence
  Primer
gctgggaattgaacgtagga

SEQ ID NO: 12 - DNA, 20 bp, Artificial Sequence
  Primer
gaggagacaaaggcaagcac

SEQ ID NO: 13 - DNA, 20 bp, Artificial Sequence
  Primer
gtgcccagtcatagccgaat

SEQ ID NO: 14 - DNA, 20 bp, Artificial Sequence
  Primer
cggagaacctgcgtgcaatc

SEQ ID NO: 15 - DNA, 25 bp, Artificial Sequence
  Primer
tcccggacaagacgggaacatgtgg

SEQ ID NO: 16 - DNA, 20 bp, Artificial Sequence
  Primer
cggagaacctgcgtgcaatc

SEQ ID NO: 17 - DNA, 22 bp, Artificial Sequence
  Primer
tgtcgcctactacctgttgtgc

SEQ ID NO: 18 - DNA, 20 bp, Artificial Sequence
  Primer
aactgcaacctttcgaagcc

SEQ ID NO: 19 - DNA, 20 bp, Artificial Sequence
  Primer
ccagctccaagaaaggacga

SEQ ID NO: 20 - DNA, 20 bp, Artificial Sequence
  Primer
tggatggcaaaggcagtgta

SEQ ID NO: 21 - DNA, 21 bp, Artificial Sequence
  Primer
gctttcctgatggttttggtg

SEQ ID NO: 22 - DNA, 20 bp, Artificial Sequence
  Primer
aggctttcttgttcctgagg